(19) United States
(12) Patent Application Publication
(10) Pub. No.: US 2004/0049805 A1
(43) Pub. Date: Mar. 11, 2004

(54) Method for the expression of biosynthetic genes in plant seeds using multiple expression constructs

(76) Inventors: Jens Lerchl, Svalov (SE); Elke Duwenig, Ludwigshafen (DE); Friedrich Bischoff, Mainz (DE); Ernst Heinz, Hamburg (DE); Hjordis Drexler, Hamburg (DE); Jodi Scheffler, Oxford, MS (US)

Correspondence Address: NIXON & VANDERHYE, PC, 1100 N GLEBE ROAD, 8TH FLOOR, ARLINGTON, VA, 22201-4714, US

(21) Appl. No.: 10/250,821

(22) PCT Filed: Jan. 18, 2002
(86) PCT No.: PCT/EP02/00461

(30) Foreign Application Priority Data
Jan. 19, 2001 (DE) 101 02 338.3

(51) Int. Cl. A01H 1/00; C12N 15/82; C12N 5/04

(52) U.S. Cl. 800/281; 435/468; 435/419; 435/320.1

(57) ABSTRACT

The present invention relates to expression cassettes, to their combination and vectors comprising the expression cassettes comprising plant promoters with an expression specificity for plant seeds, in particular linseed, and to the use of these expression cassettes or vectors for the recombinant expression of heterologous genes in plants. The invention furthermore relates to transgenic plants which have been transformed with these expression cassettes or vectors, to cultures, parts or transgenic propagation material derived therefrom, and to their use as food, feed or seed, pharmaceuticals, fine chemicals or as industrial feedstock.

The invention relates to expression cassettes, to their combination and vectors comprising the expression cassettes which comprise plant promoters with an expression specificity for plant seeds, in particular linseed, and to the use of these expression cassettes or vectors for the recombinant expression of heterologous genes in plants. The invention further relates to transgenic plants transformed with these expression cassettes or vectors, to cultures, parts or transgenic propagation material derived therefrom, and to their use as food, feed or seed, pharmaceuticals, fine chemicals or industrial feedstock.

The expression cassettes according to the invention are advantageously used in a method for the production of unsaturated fatty acids with at least two double bonds and/or a method for the production of triglycerides with an increased content of polyunsaturated fatty acids with at least two double bonds. The nucleic acid sequences SEQ ID NO: 1, SEQ ID NO: 3, SEQ ID NO: 5 or 11, which are expressed using the expression cassettes, are advantageously used in the method. These abovementioned nucleic acids are suitable in the method and for generating a transgenic organism, preferably a transgenic plant or a transgenic microorganism, with an increased content of fatty acids, oils or lipids with unsaturated C₁₈-, C₂₀- or C₂₂-fatty acids. Moreover, it is possible, with the aid of the expression cassettes according to the invention, to express in organisms, preferably plants, further genes in addition to the nucleic acid sequences SEQ ID NO: 1, SEQ ID NO: 3, SEQ ID NO: 5 and SEQ ID NO: 11 or their homologs, derivatives or analogs and gene constructs encompassing these genes or their homologs, derivatives or analogs, and their use alone or in combination with further biosynthesis genes, preferably biosynthesis genes for polyunsaturated fatty acids as shown advantageously in SEQ ID NO: 7 and SEQ ID NO: 9.

A series of products and by-products of naturally occurring metabolic processes in microorganisms, animal cells and plant cells has utility for a wide array of industries, including the feed, food, cosmetics and pharmaceutical industries. These molecules, which are collectively termed “fine chemicals”, also include, for example, lipids and fatty acids, one representative class of which are the polyunsaturated fatty acids. Fatty acids and triglycerides have a multiplicity of uses in the food industry, in animal nutrition, in cosmetics and in the pharmacological sector. Depending on whether they take the form of free saturated or unsaturated fatty acids or else triglycerides with an increased content of saturated or unsaturated fatty acids, they are suitable for a variety of uses; thus, for example, polyunsaturated fatty acids (PUFAs) are added to baby formula for increasing the nutritional value. Moreover, PUFAs have a positive effect on the cholesterol level in the blood of humans and are therefore useful for protection against heart disease. Thus, they are used in a variety of dietetic foods or in medicaments.

Microorganisms which are particularly useful for the production of PUFAs are microorganisms such as Thraustochytria or Schizochytria strains, algae such as Phaeodactylum tricornutum or Crypthecodinium species, ciliates such as Stylonychia or Colpidium, fungi such as Mortierella, Entomophthora or Mucor. Through strain selection, a number of mutant strains of the respective microorganisms have been developed which produce an array of desirable compounds including PUFAs. However, the selection of strains in which the production of a particular molecule is improved is a time-consuming and difficult process.

Alternatively, fine chemicals can conveniently be produced via producing, on a large scale, plants which have been developed in such a way that they produce the abovementioned PUFAs. Particularly well suited plants for this purpose are oil crop plants which comprise large amounts of lipid compounds, such as oilseed rape, canola, linseed, soybean, sunflower, borage and evening primrose. However, other useful plants comprising oils or lipids and fatty acids are also well suited as mentioned in the detailed description of this invention. By means of conventional breeding, an array of mutant plants has been developed which produce a spectrum of desirable lipids and fatty acids, cofactors and enzymes. However, the selection of novel plant cultivars in which the production of a particular molecule is improved is a time-consuming and difficult process or indeed impossible if the compound does not occur naturally in the respective plant, as is the case of polyunsaturated C₁₈-, C₂₀-fatty acids and C₂₂-fatty acids and those with longer carbon chains.

Owing to the positive properties of unsaturated fatty acids, there has been no lack of attempts in the past to make available genes which are involved in the synthesis of fatty acids or triglycerides for the production, in various organisms, of oils with a modified content of unsaturated fatty acids. Thus, WO 91/13972 and its US equivalent describe a Δ9-desaturase. A Δ15-desaturase is claimed in WO 93/11245 and a Δ12-desaturase is claimed in WO 94/11516. Δ6-desaturases are described in WO 93/06712, U.S. Pat. No. 5,614,393, WO 96/21022 and WO 99/27111. Further desaturases are described, for example, in EP-A-0 550 162, WO 94/18337, WO 97/30582, WO 97/21340, WO 95/18222, EP-A-0 794 250, Stukey et al., J. Biol. Chem., 265, 1990: 20144-20149, Wada et al., Nature 347, 1990: 200-203 or Huang et al., Lipids 34, 1999: 649-659. A Δ6-palmitoyl ACP desaturase is described and claimed in WO 96/13591. However, the biochemical characterization of the various desaturases is incomplete as yet since the enzymes, being membrane-bound proteins, can only be isolated and characterized with great difficulty (McKeon et al., Methods in Enzymol. 71, 1981: 12141-12147, Wang et al., Plant Physiol. Biochem., 26, 1988: 777-792).

In yeasts, both a shift in the fatty acid spectrum toward unsaturated fatty acids and an increase in productivity have been found (see Huang et al., Lipids 34, 1999: 649-659, Napier et al., Biochem. J., Vol. 330, 1998: 611-614). However, the expression of the various desaturases in transgenic plants did not show the desired success. While a shift in the fatty acid spectrum toward unsaturated fatty acids was demonstrated, it emerged that the synthesis performance of the transgenic plants dropped drastically, i.e. only smaller amounts of oils were isolated compared with the original plants.

Neither yeasts nor plants naturally produce polyunsaturated C₂₀-and/or C₂₂-fatty acids with at least two double bonds in the fatty acid molecule, such as arachidonic acid (ARA) and/or eicosapentaenoic acid (EPA) and/or docosahexaenoic acid (DHA).

This is why there still exists a great demand for novel genes which encode enzymes which are involved in the biosynthesis of unsaturated fatty acids and which make possible their production on an industrial scale. None of the prior-art biotechnological methods for the production of polyunsaturated fatty acids yields the abovementioned fatty acids in economically useful quantities.

When expressing genes in plants, there are always problems, that is to say the expression does not lead to the expected increase in the production of the desired product of interest.

Various methods are known for introducing genes into the genome of plants (Halford N G, Shewry P R, Br. Med. Bull., 2000; 56: 62-73). The aim is to generate plants with advantageous, novel properties, for example the increase in agricultural productivity, the increase in the quality in foods, or the production of specific chemicals or pharmaceuticals (Dunwell J M, J. Exp. Bot., 2000: 51 Spec No: 487-96).

A basic prerequisite for the transgenic expression of specific genes is the provision of plant-specific promoters. Promoters are important tools in plant biotechnology in order to govern the expression of a specific gene in a transgenic plant in a location- and time-specific manner and thus to exploit, or achieve in the first place, specific characteristics of the plant. A variety of promoters are known for various plant species, specific plant tissues and developmental stages.

Promoters which are used are, for example, constitutive promoters such as the Agrobacterium nopaline synthase promoter, the TR twin promoter, or the promoter of the cauliflower mosaic virus ³⁵S transcript (CaMV) (Odell et al., Nature 1985: 313,810-812), the Agrobacterium OCS (octopine synthase) promoter, the ubiquitin promoter (Holtorf S et al., Plant Mol. Biol. 1995, 29:637-646), the promoters of the vacuolar ATPase subunits, or the promoter of a prolin-rich wheat protein (WO 91/13991). The disadvantage of these promoters is that they are constitutively active in virtually all tissues of the plant. A targeted expression of genes in specific plant parts or at specific points in time of development is not possible when these promoters are used.

Promoters whose activity is regulated in a tissue-specific or development-dependent manner have been isolated. Specificities have been described for the anthers, ovaries, flowers, leaves, stems, roots and seeds. The stringency of the specificity and the expression activity of these promoters differ greatly. Promoters which must be mentioned are those which ensure leaf-specific expression, such as the potato cytosolic FBPase promoter (WO 97/05900), the Rubisco (ribulose-1,5-bisphosphate carboxylase) SSU (small subunit) promoter, or the potato ST-LSI promoter (Stockhaus et al., EMBO J. 8 (1989), 244-245).

Further promoters are, for example, specific promoters for tubers, storage roots or roots, such as, for example, the class I patatin promoter (B33), the potato cathepsin D inhibitor promoter, the starch synthase (GBSS1) promoter or the sporamin promoter, fruit-specific promoters such as, for example, the tomato fruit-specific promoter (EP-A 409625), fruit-maturation-specific promoters such as, for example, the tomato fruit-maturation-specific promoter (WO 94/21794), flower-specific promoters, such as the phytoene synthase promoter (WO 92/16635), or the promoter of the P-rr gene (WO 98/22593).

A variation in the activity of a promoter as a function of the developmental stage of the plant has been described, inter alia, by Baerson et al. (Baerson S R, Lamppa G K. Plant Mol. Biol. 1993;22(2):255-67).

Seed-specific promoters are of particular interest owing to the importance of the seed as one of the main sources of food or feed for humans and animals and as a production site for substances of interest. Promoters which govern an expression in seeds and plant embryos are known. Thus, for example, the promoters of genes have been identified which encode storage proteins of various dicots. Seed-specific promoters are, for example, the phaseolin promoter (U.S. Pat. No. 5,504,200, Bustos M M et al., Plant Cell. 1989;1(9):839-53), the promoter of the 2S albumin gene (Joseffson L G et al., J Biol Chem 1987, 262:12196-12201), the legumin gene (Shirsat A et al., Mol Gen Genet. 1989;215(2):326-331), the USP (unknown seed protein) promoter (Baumlein H et al., Molecular & General Genetics 1991, 225(3):459-67), the napin gene (Stalberg K, et al., L. Planta 1996, 199:515-519), the sucrose binding protein (WO 00/26388), or the LeB4 promoter (Baumlein H et al., Mol Gen Genet 1991, 225: 121-128). They govern a high, seed-specific expression of storage proteins.

Despite the general assumption that plant promoters can be transferred from one species to the other and also exhibit similar activities and specificities in different plant species, evidence has been accumulating that this assumption suffers from limitations. Thus, it emerged that the level of the transgenic expression of heterologous genes under the control of these promoters is frequently greatly dependent on the host plant species. It has been found that the expression is not always totally cell-type-specific. Differences in the expression pattern, and the expression level, of a specific promoter can be caused by different host plants or by different insertion sites into the genome of the host plant (Goossens A et al., Plant Phys 1999, 120:1095-1104).

The use of a promoter in another plant species can be greatly limited owing to a gene expression in other plant parts, for example when the expression of the gene engages in the metabolism of the cell, the composition of the membrane lipids, or biosynthesis.

It is an object of the present invention to provide further expression cassettes for the expression in plants and to use them for the expression of genes, advantageously biosynthesis genes, alone or optionally in combination with other enzymes in a method, for example for the production of polyunsaturated fatty acids.

We have found that this object is achieved by the expression cassette according to the invention with a structure selected from the group consisting of:

a) L1-promoter -structural gene-L2,

b) L1-promoter-structural gene-L2-L1-promoter-structural gene-L2,

c) L1-promoter-structural gene-L2-L1-promoter-structural gene-L2-L1-promoter-structural gene-L2,

where L1, L2, promoter and structural gene have the following meanings:

L1=SEQ ID NO: 32 or a sequence comprising equivalent restriction cleavage sites,

L2=independently of one another SEQ ID NO: 33, 34 or 35 or sequences comprising equivalent restriction cleavage sites,

promoter=plant promoter

structural gene =a nucleic acid sequence which can be expressed in plants.

The structural gene is advantageously a biosynthesis gene, preferably a biosynthesis gene of the lipid or fatty acid metabolism, advantageously a plant gene. In an especially advantageous use form, the structural gene is a nucleic acid sequence which encodes proteins selected from the group consisting of:

acyl-CoA dehydrogenase(s), acyl-ACP[=acyl carrier protein] desaturase(s), acyl-ACP thioesterase(s), fatty acid acyl transferase(s), fatty acid synthase(s), fatty acid hydroxylase(s), acetyl-coenzyme A carboxylase(s), acyl-coenzyme A oxidase(s), fatty acid desaturase(s), fatty acid acetylenases, lipoxygenases, triacylglycerol lipases, allenoxide synthases, hydroperoxide lyases or fatty acid elongase(s).

Very especially preferably, the structural gene is a nucleic acid sequence selected from the group consisting of:

a) a nucleic acid sequence with the sequence shown in SEQ ID NO: 1, SEQ ID NO: 3, SEQ ID NO: 5 or SEQ ID NO: 11,

b) nucleic acid sequences which, owing to the degeneracy of the genetic code, are obtained by backtranslating the amino acid sequences shown in SEQ ID NO: 2, SEQ ID NO: 4, SEQ ID NO: 6 or SEQ ID NO: 12,

c) derivatives of the nucleic acid sequence shown in SEQ ID NO: 1, SEQ ID NO: 3, SEQ ID NO: 5 or SEQ ID NO: 11, which encode polypeptides with the amino acid sequences shown in SEQ ID NO: 2, SEQ ID NO: 4, SEQ ID NO: 6 or SEQ ID NO: 12 and have at least 50% homology at the amino acid level, without essentially reducing the enzymatic action of the polypeptides.

A sequence comprising equivalent restriction cleavage sites is to be understood as meaning, for the purposes of the invention, sequences comprising restriction cleavage sites which are suitable for the construction of multiple expression cassettes, that is to say which are suitably not present in the structural gene or in the binary vector. Such restriction cleavage sites such as, for example, EcoRI, BamHI, SacI, PstI, NcoI, NdeI, BglI, BglII, XhoI, Xba and others are known to the skilled worker and can be found in specialist textbooks.

The expression cassettes according to the invention can be used for the expression of genes in economically important crop plants such as, for example, linseed, for which no endogenous seed-specific promoters were known. As shown in existing publications, linseed is especially problematic for a seed-specific expression of genes since it seems that a plurality of promoters which are used routinely by the skilled worker for the seed-specific expression in other plants do not work in, for example, other plants such as linseed, that is to say do not lead to a transcription and, eventually, expression of the mRNA structural gene.

The invention also provides for the use of the above-mentioned expression cassettes in a method for the production of fatty acid esters with an increased content of polyunsaturated fatty acids with at least two double bonds, which comprises introducing, into a fatty-acid-ester-producing organism, at least one nucleic acid sequence selected from the group consisting of

a) a nucleic acid sequence with the sequence shown in SEQ ID NO: 1, SEQ ID NO: 3, SEQ ID NO: 5 or SEQ ID NO: 11,

b) nucleic acid sequences which, owing to the degeneracy of the genetic code, are obtained by backtranslating the amino acid sequences shown in SEQ ID NO: 2, SEQ ID NO: 4, SEQ ID NO: 6 or SEQ ID NO: 12,

c) derivatives of the nucleic acid sequence shown in SEQ ID NO: 1, SEQ ID NO: 3, SEQ ID NO: 5 or SEQ ID NO: 11, which encode polypeptides with the amino acid sequences shown in SEQ ID NO: 2, SEQ ID NO: 4, SEQ ID NO: 6 or SEQ ID NO: 12 and have at least 50% homology at the amino acid level, without essentially reducing the enzymatic action of the polypeptides,

growing the organism, and isolating the fatty acid esters present in the organism.

The nucleic acid sequences used in the method are isolated nucleic acid sequences which encode polypeptides with Δ5-, Δ6- or Δ12-desaturase activity.

It is advantageous to produce fatty acid esters with polyunsaturated C₁₈-, C₂₀- and/or C₂₂-fatty acid molecules with at least two double bonds in the fatty acid ester by the method. These fatty acid molecules preferably comprise three, four or five double bonds and advantageously lead to the synthesis of arachidonic acid (ARA), eicosapentaenoic acid (EPA) or docosahexaenoic acid (DHA).

The fatty acid esters with polyunsaturated C₁₈-, C₂₀- and/or C₂₂-fatty acid molecules can be isolated from the organisms used for the production of the fatty acid esters in the form of an oil or lipid for example in the form of compounds such as sphingolipids, phosphoglycerides, lipids, glycolipids, phospholipids, monoacyl glycerides, diacyl glycerides, triacyl glycerides or other fatty acid esters comprising the polyunsaturated fatty acids with at least two double bonds.

Suitable organisms for the production by the method are, in principle, all prokaryotic or eukaryotic organisms such as prokaryotic or eukaryotic microorganisms such as Gram-positive or Gram-negative bacteria, fungi, yeasts, algae, ciliates, animal or plant cells, animals or plants such as mosses, dicotyledonous or monocotyledonous plants. It is advantageous to use, in the method according to the invention, organisms which belong to the oil-producing organisms, that is to say which are used for the production of oils, such as microorganisms such as Crypthecodinium, Thraustochytrium, Phaeodactylum and Mortierella, Entomophthora, Mucor, and other algae or fungi, and animals or plants, in particular plants, preferably oil crop plants which contain large amounts of lipid compounds, such as soybean, peanut, oilseed rape, canola, sunflower, safflower, evening primrose, linseed, borage, trees (oil palm, coconut) or crops such as maize, wheat, rye, oats, triticale, rice, barley, cotton, cassaya, pepper, Tagetes, solanaceous plants such as potato, tobacco, aubergine and tomato, Vicia species, pea, alfalfa or bush plants (coffee, cacao, tea), Salix species, and also perennial grasses and fodder crops. Plants according to the invention which are especially preferred are oil crop plants such as soybean, peanut, oilseed rape, canola, linseed, evening primrose, sunflower, safflower or trees (oil palm, coconut).

The method comprises either breeding a suitable transgenic organism or transgenic microorganism or breeding transgenic plant cells, tissues, organs or intact plants comprising the nucleotide sequences according to the invention of SEQ ID NO: 1, 3, 5 or 11, if appropriate in connection with the sequences shown in SEQ ID NO: 7 and/or SEQ ID NO: 9 alone or in combination with sequences of advantageous expression cassettes according to the invention in advantageous vectors with SEQ ID NO: 13-17 or their homologs, derivatives or analogs or a gene construct which encompasses SEQ ID NO: 1, 3, 5 or 11, if appropriate in connection with SEQ ID NO: 7 and/or 9 or their homologs, derivatives or analogs, or a vector comprising this sequence or the gene construct which brings about the expression of nucleic acid molecules according to the invention, so that a fine chemical is produced. In a preferred embodiment, the process furthermore comprises the step of obtaining a cell comprising such nucleic acid sequences according to the invention, wherein a cell is transformed with a desaturase nucleic acid sequence, a gene construct or a vector which bring about the expression of a desaturase nucleic acid according to the invention, alone or in combination. In a further preferred embodiment, this method furthermore comprises the step of obtaining the fine chemical from the culture. In an especially preferred embodiment, the cell belongs to the order of the ciliates, to microorganisms such as fungi, or to the plant kingdom, in particular to oil crop plants; especially preferred are microorganisms or oil crop plants, for example peanut, oilseed rape, canola, linseed, soybean, safflower (thistle), sunflowers or borage.

Transgenic/recombinant is to be understood as meaning, for the purposes of the invention, that the nucleic acids used in the method or the expression cassettes according to the invention, are not at their natural location in the genome of an organism; it is possible here for the nucleic acids to be expressed homologously or heterologously. However, transgenic/recombinant also means that the nucleic acids or expression cassettes are at their natural location in the genome of an organism, but that the sequence has been modified over the natural sequence and/or that the regulatory sequences of the natural sequences have been modified. Transgenic/recombinant preferably describes the expression of the nucleic acids according to the invention at an unnatural location in the genome, that is to say a homologous or, preferably, heterologous expression of the nucleic acids exists. Preferred transgenic organisms are the abovementioned transgenic plants, preferably oil crop plants.

The polyunsaturated fatty acids contained in the fatty acid esters produced by the method can be liberated, for example, via treatment with alkali such as aqueous KOH or NaOH, advantageously in the presence of an alcohol such as methanol or ethanol, and can be isolated via, for example, phase separation and subsequent acidification with, for example, H₂SO₄.

The fatty acid esters produced by the method are obtained in the form of oils, lipids and/or fatty acids containing at least two double bonds in the fatty acid molecules, preferably three, four, five or six double bonds. A further possible application of the abovementioned substances is also compositions comprising the abovementioned oils, lipids and/or fatty acids, and the use of the oils, lipids and/or fatty acids or of the compositions in feed, foodstuffs, cosmetics or pharmaceuticals.

A further aspect relates to a method of modulating the production of a molecule by a microorganism. This method encompasses the contacting of the cell with a substance which modulates the desaturase activity according to the invention alone or in combination or the desaturase nucleic acid expression, so that a cell-associated activity is modified in relation to the same activity in the absence of the substance. In a preferred embodiment, one or two metabolic pathway(s) of the cell for lipids and fatty acids, cofactors and enzymes is/are modulated, or the transport of compounds through these membranes is modulated, so that the yield or the production rate of a desired fine chemical by this microorganism is improved. The substance which modulates the desaturase activity can be a substance which stimulates the desaturase activity or desaturase nucleic acid expression or which can be used as intermediate in fatty acid biosynthesis. Examples of substances which stimulate the desaturase activity or desaturase nucleic acid expression are, inter alia, small molecules, active desaturases and desaturase-encoding nucleic acids which have been introduced into the cell. Examples of substances which inhibit the desaturase activity or desaturase expression are, inter alia, small molecules and antisense desaturase nucleic acid molecules.

A further aspect relates to a method of modulating the yields of a desired compound from a cell, comprising introducing, into a cell, a wild-type or mutant desaturase gene which is either maintained on a separate plasmid or integrated into the genome of the host cell. Upon integration into the genome, integration can be random or can be effected by recombination in such a way that the native gene is replaced by the copy introduced, thereby modulating the production of the desired compound by the cell, or by using a gene in trans, so that the gene is linked operably to a functional expression unit comprising at least one sequence which ensures the expression of a gene and at least one sequence which ensures the polyadenylation of a functionally transcribed gene.

In a preferred form of the method, the yields are modified. In a further preferred embodiment, the desired chemical is augmented, it being possible to reduce undesired interfering compounds. In an especially preferred embodiment, the desired fine chemical is a lipid or a fatty acid, a cofactor or an enzyme. In an especially preferred embodiment, this chemical is a polyunsaturated fatty acid. More preferably, it is selected from among arachidonic acid (ARA), eicosapentaenoic acid (EPA) or docosahexaenoic aicd (DHA).

The present invention provides advantageous multiexpression cassettes and constructs for the multiparallel seed-specific expression of gene combinations in plants.

They can be used in the above-described method of expressing genes, preferably those described in SEQ ID NO: 1, SEQ ID NO: 3, SEQ ID NO: 5, SEQ ID NO: 7, SEQ ID NO: 9 or SEQ ID NO: 11, in algae and fungi and plants. Preferred organisms for the method are, in particular, oil seed plants.

By using the expression cassettes according to the invention, [lacuna] can be used in the method in conjunction with the abovementioned nucleic acid molecules for the recombinant modification of plants so that they eventually lead to the production of better or more efficient producers of one or more fine chemicals. This improved production or production efficiency of a fine chemical can be brought about by a direct effect of the manipulation of a gene according to the invention or by an indirect effect of this manipulation. Fine chemicals for the purposes of the invention are understood as being for example fatty acid esters containing polyunsaturated fatty acids with at least two double bonds, such as sphingolipids, phosphoglycerides, lipids, glycolipids, phospholipids, monoacyl glycerides, diacyl glycerides, triacyl glycerides or other fatty acid esters containing the polyunsaturated fatty acids with at least two double bonds. They are furthermore understood as being compounds such as vitamins, for example vitamin E, vitamin C, vitamin B2, vitamin B6, pantolactone, carotenoids such as astaxanthin, β-carotene, zeaxanthin and others.

Mosses and algae are the only plant systems known which produce substantial amounts of polyunsaturated fatty acids such as arachidonic acid (ARA) and/or eicosapentaenoic aicd (EPA) and/or docosahexaenoic acid (DHA). Mosses contain PUFAs in membrane lipids, while algae, organisms related to algae and some fungi also accumulate substantial amounts of PUFAs in the triacylglycerol fraction. Nucleic acid molecules which are isolated from such strains which also accumulate PUFAs in the triacylglycerol fraction are therefore particularly advantageously suitable for modifying the lipid and PUFA production system in a host, in particular in microorganisms, such as in the microorganisms mentioned above, and plants such as oil crop plants, for example oilseed rape, canola, linseed, soybean, sunflowers, borage. They can therefore be used advantageously in the method.

The nucleic acid sequences used in the method using the expression cassettes according to the invention encode desaturases which are suitable for the production of long-chain polyunsaturated fatty acids, preferably having more than sixteen, eighteen or twenty carbon atoms in the carbon skeleton of the fatty acid and/or at least two double bonds in the carbon chain, a nucleic acid encoding an enzyme capable of introducing double bonds at the Δ5 position, in another case at the Δ6 position and in a further case at the A12 position. Large amounts of PUFAs may be obtained in the triacylglycerol fraction with the aid of these nucleic acids. Furthermore, further desaturases have been isolated which, alone or together with a Δ4 desaturase, can be utilized for a method for the production of polyunsaturated fatty acids. In the application, the singular, i.e. a desaturase gene or protein, is also understood as meaning the plural, i.e. the desaturase genes or proteins.

The production of a trienoic acid with C₁₈-carbon chain with the aid of desaturases has already been demonstratead. However, in these methods known from the literature, the production of γ-linolenic acid was claimed. As yet, however, nobody has been able to demonstrate the production of very long-chain polyunsaturated fatty acids (with C₂₀ carbon chain and longer and of trienoic acids and higher unsaturated types) by modified organisms alone.

To produce the long-chain PUFAs according to the invention, the polyunsaturated C₁₈-fatty acids must first be elongated by at least two carbon atoms by the enzymatic activity of an elongase. Following an elongation cycle, this enzyme activity leads to C₂₀-fatty acids, and after two, three and four elongation cycles, to C₂₂-, C₂₄- or C₂₆-fatty acids. The nucleic acid sequences disclosed in the present invention which encode various desaturases can, in concert with elongases, lead to very long-chain polyunsaturated fatty acids. The activity of the desaturases according to the invention preferably leads to C₁₈-, C₂₀- and/or C₂₂-fatty acids with at least two double bonds in the fatty acid molecule, preferably with three, four, five or six double bonds, especially preferably to C₁₈- and/or C₂₀-fatty acids with at least two double bonds in the fatty acid molecule, preferably with three, four or five double bonds in the molecule. The elongation of the fatty acid can be effected by combining the desaturases according to the invention with an elongase activity, it being possible to use the elongase encoded by SEQ ID NO: 9 in an advantageous fashion. After the elongation by the enzyme(s) according to the invention has taken place, further desaturation steps such as, for example, a desaturation at the Δ5 position, may take place. The combination with other elongases such as those which lead to an elongation from C₁₈- to C₂₀-chains or from C₂₀- to C₂₂₋₂₄-chains as disclosed in WO 00/12720 may also be used and/or a desaturase with activity for the Δ4 position can advantageously be employed in order to obtain the highly desaturated fatty acids. The products of the desaturase activities and the possible further desaturation therefore lead to preferred PUFAs with a higher degree of desaturation, such as dihomo-γ-linolenic acid, docosadienoic acid, arachidonic acid, ω6-dicosatrienedihomo-γ-linolenic acid, eicosapentaenoic acid, ω3-eicosatrienoic acid, ω3-eicosatetraenoic acid, docosapentaenoic acid or docosahexaenoic acid. Substrates of the enzyme activity according to the invention are, for example, taxoleic acid; 6,9-octadecadienoic acid, linoleic acid, pinolenic acid, α- or γ-linolenic acid or stearidonic acid and arachidonic acid, eicosatetraenoic acid, docosopentaenoic acid, eicosapentaenoic acid. Preferred substrates are linoleic acid, γ-linolenic acid and/or α-linolenic acid and arachidonic acid, eicosatetraenoic acid, docosapentaenoic acid, eicosapentaenoic acid. Especially preferred products of the process according to the invention are arachidonic acid, docosapentaenoic acid, eicosapentaenoic acid. The C₁₈-fatty acids with at least two double bonds in the fatty acid can be elongated by the enzymatic activity according to the invention in the form of the free fatty acid or in the form of the esters, such as phospholipids, glycolipids, sphingolipids, phosphoglycerides, monoacyl glycerides, diacyl glycerides or triacyl glycerides.

Of particular importance for human nutrition is the conjugated linoleic acid “CLA”. CLA is understood as meaning in particular fatty acids such as C18:2^{9 cis, litrans} or the isomer C18:2 ^{10trans, 12 cis}, which, once taken up, can be desaturated or elongated in the body owing to human enzyme systems and can contribute to health-promoting effects. The desaturases according to the invention (Δ12-desaturase) also allow those conjugated fatty acids with at least two double bonds in the molecule to be desaturated and thus allow such health-promoting fatty acids to be made available for human nutrition. Further examples of conjugated fatty acids are α-parinaric acid, punicic acid, eleostearic acid and calendulic acid.

Using the expression cassettes according to the invention in cloning vectors in plants and in the transformation of plants like those which are published and cited in: Plant Molecular Biology and Biotechnology.(CRC Press, Boca Raton, Fla.), Chapter 6/7, pp. 71-119 (1993); F. F. White, Vectors for Gene Transfer in Higher Plants; in: Transgenic Plants, Vol. 1, Engineering and Utilization, Eds.: Kung and R. Wu, Academic Press, 1993, 15-38; B. Jenes et al., Techniques for Gene Transfer, in: Transgenic Plants, Vol. 1, Engineering and Utilization, Eds.: Kung and R. Wu, Academic Press (1993), 128-143; Potrykus, Annu. Rev. Plant Physiol. Plant Molec. Biol. 42 (1991), 205-225)), genes, advantageously biosynthesis genes such as the nucleic acids described above, can be used for the recombinant modification of a broad spectrum of plants so that these plants become a better or more efficient producer for example of one or more lipid-derived products, such as PUFAs. This improved production or production efficiency of a for example lipid-derived product, such as PUFAs, can be brought about by a direct action of the manipulation or an indirection action of this manipulation.

A series of mechanisms exist by means of which the modification of a desaturase protein according to the invention can have a direct effect on the yield, production and/or production efficiency of a fine chemical from an oil crop plant or a microorganism, owing to a modified protein. The number or activity of the desaturase protein or desaturase gene and of gene combinations of desaturases and elongases can be increased, so that larger amounts of these compounds are produced de novo since the organisms lacked this activity and ability to biosynthesize them prior to introduction of the gene in question. This also applies analogously to the combination with further desaturases or elongases or further enzymes of the lipid metabolism. The use of various divergent sequences, i.e. sequences which differ at the DNA sequence level, may also be advantageous, or else the use of promoters for gene expression which makes possible a different temporal gene expression, for example as a function of the degree of maturity of the seed or oil-storing tissue.

The introduction of a desaturase gene, of several saturase genes under the control of the expression cassettes according to the invention into an organism, alone or in combination with other genes in a cell, can not only increase the biosynthesis flux toward the end product, but also increase, or generate de novo, the corresponding composition of the end products, for example the triacylglycerols. Likewise, the number or activity of other genes which participate in the import of nutrients required for the biosynthesis of one or more fine chemicals (for example fatty acids, polar and neutral lipids) can be increased, so that the concentration of the precursors, cofactors or intermediates within the cells or within the storage compartment is increased, thus further increasing the ability of the cells to produce PUFAs as described hereinbelow. Fatty acids and lipids themselves are desirable as fine chemicals; by optimizing the activity or increasing the number of one or more desaturases which participate in the biosynthesis of these compounds, or by destroying the activity of one or more desaturases which participate in the breakdown of these compounds, it can be possible to increase the yield, production and/or production efficiency of fatty acid and lipid molecules from plants or microorganisms.

The mutagenesis of the desaturase gene(s) according to the invention may furthermore lead to a desaturase protein with modified activities which have a direct or indirect effect on the production of one or more desired fine chemicals. For example, the number or activity of the desaturase gene(s) according to the invention may be increased so that the normal metabolic wastes or by-products of the cell (possibly increased in quantity due to the overproduction of the desired fine chemical) are exported efficiently before they can damage other molecules or processes within the cell (which would decrease the viability of the cell) or to interfere with the fine chemical biosynthetic pathways (which would decrease the yield, production or production efficiency of the desired fine chemical). Furthermore, the relatively large intracellular quantities of the desired fine chemical may themselves be toxic to the cell or interfere with enzyme feedback mechanisms, such as allosteric regulation; for example, by increasing the activity or number of other downstream enzymes or detoxification enzymes of the PUFA pathway, it might increase the allocation of the PUFA to the triacylglycerol fraction; the viability of seed cells might be increased, in turn leading to better development of cultured cells or to seeds which produce the desired fine chemical. The desaturase according to the invention may also be manipulated in such a way that the relative amounts of the various lipid and fatty acid molecules are produced. This may have a profound effect on the lipid composition of the membrane of the cell and generates novel oils in addition to the occurrence of newly-synthesized PUFAs. Since each type of lipid has different physical properties, a change in the lipid composition of a membrane may significantly alter membrane fluidity. Changes in membrane fluidity can have an effect on the transport of molecules across the membrane and on the integrity of the cell, both of which have a profound effect on the production of fine chemicals. In plants, these changes may additionally impact on other traits, such as tolerance to abiotic and biotic stress situations.

In the method, use can be made of isolated nucleic acid molecules (for example cDNAs) encompassing nucleotide sequences which encode one desaturase or several desaturases or biologically active parts thereof, or nucleic acid fragments which are suitable as primers or hybridization probes for detecting or amplifying desaturase-encoding nucleic acids (for example DNA or mRNA). In especially preferred embodiments, the nucleic acid molecule encompasses one of the nucleotide sequences shown in sequence ID NO:1 or 3 and 5 or the coding region or a complement of one of these nucleotide sequences. In other especially preferred embodiments, the isolated nucleic acid molecule encompasses a nucleotide sequence which hybridizes with a nucleotide sequence as shown in the sequence SEQ ID NO: 1, 3, 5 or 11 or a portion thereof or which has at least 50% homology, preferably at least approxiately 60% homology, more preferably at least approximately 70%, 80% or 90% homology and very especially preferably at least approximately 95%, 96%, 97%, 98%, 99% or more homology therewith. In other preferred embodiments, the isolated nucleic acid molecule encodes one of the amino acid sequences shown in sequence SEQ ID NO: 2, 4, 6 or 12. The preferred desaturase gene preferably also has at least one of the desaturase activities described herein.

In a further embodiment, the isolated nucleic acid molecule encodes a protein or a portion thereof, the protein or the portion thereof comprising an amino acid sequence which has sufficient homology with an amino acid sequence of the sequence SEQ ID NO: 2, 4, 6 or 12 that the protein or the portion thereof retains a desaturase activity. Preferably, the protein or the portion thereof encoded by the nucleic acid molecule retains the ability of participating in the metabolism of compounds required for the synthesis of cell membranes of plants or in the transport of molecules across these membranes. In one embodiment, the protein encoded by the nucleic acid molecule has at least approximately 50% homology, preferably at least approximately 60% homology, more preferably at least approximately 70%, 80% or 90% and very especially preferably at least approximately 95%, 96%, 97%, 98%, 99% or more homology with an amino acid sequence of the sequence SEQ ID NO: 2, 4, 6 or 12. In a further preferred embodiment, the protein is a full-length protein which is essentially in parts homologous to a complete amino acid sequence of SEQ ID NO: 2, 4, 6 or 12 (which is derived from the open reading frame shown in SEQ ID NO: 1, 3, 5 or 11).

In other embodiments, the isolated desaturase encompasses an amino acid sequence which has at least approximately 50% homology with one of the amino acid sequences of SEQ ID NO: 2, 4, 6 or 12 and which can participate in the metabolism of compounds required for the synthesis of fatty acids in a microorganism or plant cell or in the transport of molecules across these membranes, desaturated C₁₈— or C₂₀₋₂₂-carbon chains being understood with double bonds in at least two positions.

In another preferred embodiment, the isolated nucleic acid molecule originates from Phaeodactylum tricornutum UTEX646 and encodes a protein (for example a desaturase fusion protein) containing a biologically active domain which has at least approximately 50% or more homology with an amino acid sequence of the sequence SEQ ID NO: 2, 4, 6 or 12 and retains the ability of participating in the metabolism of compounds required in the synthesis of cell membranes of plants or in the transport of molecules across these membranes or has at least one of the desaturation activities resulting in PUFAs such as GLA, ALA, dihomo-γ-linolenic acid, ARA, EPA or DHA or their precursor molecules, and also encompasses heterologous nucleic acid sequences which encode a heterologous polypeptide or regulatory proteins.

As an alternative, the isolated desaturase can comprise an amino acid sequence which is encoded by a nucleotide sequence which hybridizes with a nucleotide sequence of SEQ ID NO: 1, 3, 5 or 11, for example under stringent conditions, or which has at least approximately 50% homology, preferably at least approximately 60% homology, more preferably at least approximately 70%, 80% or 90% homology and even more preferably at least approximately 95%, 96%, 97%, 98%, 99% or more homology therewith. It is also preferred for the preferred desaturase forms likewise to have one of the desaturase activities described herein.

In another embodiment, the isolated nucleic acid molecule is at least 15, 25, 50, 100, 250 or more nucleotides in length and hybridizes under stringent conditions with a nucleic acid molecule comprising a nucleotide sequence of SEQ ID NO: 1, 3, 5 or 17. Preferably, the isolated nucleic acid molecule corresponds to a naturally occurring nucleic acid molecule. More preferably, the isolated nucleic acid molecule encodes naturally occurring Phaeodactylum desaturase or a biologically active portion thereof.

A further embodiment of the invention comprises expression cassettes which make possible the expression of the nucleic acids according to the invention with the sequences SEQ ID NO: 1, 3, 5 or 11 in the various organisms such as tissues, parts of plants or intact plants.

The expression cassette (=nucleic acid construct or nucleic acid fragment) according to the invention is to be understood as meaning the [lacuna] in SEQ ID NO: 32 as L1 and a promoter, a structural gene selected from among the advantageous sequences SEQ ID NO: 1, SEQ ID NO: 3, SEQ ID NO: 5 or SEQ ID NO: 11, which are the result of the genetic code and/or their functional or nonfunctional derivatives, and the polylinker-terminator-polylinker sequences (=L2) SEQ ID NO: 33, SEQ ID NO: 34 or SEQ ID NO: 35. They advantageously govern gene expression in the host cell. These regulatory sequences present in the constructs are intended to make possible the targeted expression of the genes and of protein expression. Depending on the host organism, this may mean, for example, that the gene is first induced and only then expressed and/or overexpressed, or that it is expressed and/or overexpressed immediately. For example, these regulatory sequences are sequences to which inductors or repressors bind, thus regulating the expression of the nucleic acid. In addition to these novel regulatory sequences, or instead of these sequences, the natural regulation of these sequences before the actual structural genes may still be present and, if appropriate, may have been genetically modified so that the natural regulation has been eliminated and gene expression increased. However, the gene construct may also have a simpler construction, that is to say no additional regulatory sequences were inserted before the nucleic acid sequence or its derivatives, and the natural promoter together with its regulation has not been removed. Instead, the natural regulatory sequence was mutated in such a way that regulation no longer takes place and/or gene expression is increased. These modified promoters can be inserted before the natural gene in the form of part-sequences (=promoter with parts of the nucleic acid sequences according to the invention) or else alone, in order to increase the activity. Moreover, the gene construct can additionally advantageously also comprise one or more of what are known as enhancer sequences linked functionally to the promoter, and these make possible an increased expression of the nucleic acid sequence. Additional advantageous sequences, such as further regulatory elements or terminators, may also be inserted at the 3′ end of the DNA sequences. The Δ5 desaturase/Δ6 desaturase and/or Δ12 desaturase genes may be present in the expression cassette (=gene construct) in one or more copies.

As described above, the regulatory sequences or factors can preferably have a positive effect on the gene expression of the introduced gene, thus increasing it. Thus, an enhancement of the regulatory elements can advantageously take place at transcription level, by using strong transcription signals such as promoters and/or enhancers. In addition, however, enhanced translation is also possible, for example by improving the stability of the mRNA.

A further aspect of the invention relates to vectors, for example recombinant expression vectors, comprising at least one of the expression cassettes according to the invention, and to host cells into which the expression cassettes according to the invention or these vectors have been introduced, in particular microorganisms, plant cells, plant tissues, plant organs or intact plants. In one embodiment, such a host cell can store fine chemical compounds, in particular PUFAs; to isolate the desired compound, the cells are harvested. The compound (oils, lipids, triacyl glycerides, fatty acids) or the desaturase can then be isolated from the medium or from the host cell which, in the case of plants, are cells comprising or storing the fine chemicals, most preferably cells of storage tissues such as seed coats, tubers, epidermis cells and seed cells, endosperm or embryo tissue.

Yet another aspect of the invention relates to a genetically modified transgenic plant, preferably an oil crop plant as mentioned above, especially preferably an oilseed rape or linseed plant into which an expression cassette according to the invention which advantageously contains further genes such as desaturase gene has been introduced. In one embodiment, the genome of oilseed rape or linseed has been modified by introducing, as transgene, an expression cassette according to the invention advantageously containing further nucleic acid molecules, which encodes for example a wild-type or mutated desaturase sequence. In a preferred embodiment, oilseed rape or linseed is also used for the production of a desired compound such as lipids and fatty acids, with PUFas being especially preferred.

In yet another preferred embodiment, the moss Physcomitrella patens can be used for demonstrating the function of an expression cassette having a desaturase gene using homologous recombination on the basis of the nucleic acids described in the present invention.

The desaturase polypeptide or a biological active part thereof can, under the control of the expression cassette according to the invention, advantageously be linked functionally to a further polypeptide which has an enzymatic activity other than the desaturases, for example an elongase, acyltransferase or other activity, to form a fusion protein. This fusion protein advantageously has an activity which differs from that of the desaturase alone. In other preferred embodiments, this fusion protein participates in the metabolism of compounds which are required for the synthesis of lipids and fatty acids, cofactors and enzymes in microorganisms or plants, or in the transport of molecules via these membranes. Especially preferably, the introduction of this fusion protein into a host cell modulates the production of a desired compound within a cell and by the cell. In a preferred embodiment, these fusion proteins also contain Δ4-, Δ5- or Δ6-, Δ8-, Δ15-, Δ17- or Δ19-desaturase activities, alone or in combination. Preferred embodiments are, in particular, those gene combinations which are selected from among SEQ ID NO: 7 or 9, or parts thereof, derivatives or their homologs. Particularly preferred are those combinations which contain the complete protein activity as in SEQ ID NO: 1, 3, 5 or 11 and, inserted into multiexpression cassettes defined by SEQ ID NO: 13, 14, 15, 16 and 17, are suitable for the transformation of plants and expression in plants.

### DETAILED DESCRIPTION OF THE INVENTION

The invention also relates to the expression cassettes in combination with (an) isolated nucleic acid sequence(s) encoding a polypeptide with desaturase activity selected from the group consisting of

a) a nucleic acid sequence with the sequence shown in SEQ ID NO:

1, SEQ ID NO: 3, SEQ ID NO: 5 or SEQ ID NO: 11,

b) nucleic acid sequences which, owing to the degeneracy of the genetic code, are obtained by backtranslating the amino acid sequences shown in SEQ ID NO: 2, SEQ ID NO: 4, SEQ ID NO: 6 or SEQ ID NO: 12,

c) derivatives of the nucleic acid sequence shown in SEQ ID NO: 1, SEQ ID NO: 3, SEQ ID NO: 5 or SEQ ID NO: 11, which encode polypeptides with the amino acid sequences shown in SEQ ID NO: 2, SEQ ID NO: 4, SEQ ID NO: 6 or SEQ ID NO: 12 and have at least 50% homology at the amino acid level, without essentially reducing the enzymatic action of the polypeptides.

The invention furthermore relates to (an) amino acid sequence(s) which is/are encoded by the abovementioned nucleic acid sequence(s) (for the purposes of the invention, the singular is intended to comprise the plural and vice versa). Specifically, the invention relates to amino acid sequences encoded by the sequence shown in SEQ ID NO: 1, SEQ ID NO: 3, SEQ ID NO: 5 or SEQ ID NO: 11.

The present invention provides expression cassettes which are suitable for expressing nucleic acids and protein molecules with desaturase activity and of nucleic acids encoding proteins which participate in the metabolism of lipids and fatty acids, PUFA cofactors and enzymes in the moss Physcomitrella patens or in the transport of lipophilic compounds via membranes. The compounds according to the invention can be used for modulating the production of fine chemicals from organisms such as plants such as maize, wheat, rye, oats, triticale, rice, barley, soybean, peanut, cotton, Linum species such as linseed or flax, Brassica species such as oilseed rape, canola and turnip rape, pepper, sunflower, borage, evening primrose and Tagetes, Solanaceae plants such as potato, tobacco, egg-plant and tomato, Vicia species, pea, cassaya, alfalfa, bush plants (coffee, cacao, tea), Salix species, trees (oil palm, coconut) and perennial grasses and fodder crops, either directly (for example when the overexpression or optimization of a fatty acid biosynthesis protein has a direct effect on the yield, production and/or production efficiency of the fatty acid from modified organisms) or they can have an indirect effect which nevertheless leads to an increased yield, production and/or production efficiency of the desired compound or to a decrease in undesired compounds (for example when the modulation of the metabolism of lipids and fatty acids, cofactors and enzymes leads to changes in yield, production and/or production efficiency or the composition of the desired compound within the cells, which, in turn, may have an effect on the production of one or more fine chemicals). Aspects of the invention are illustrated in greater detail hereinbelow.

I. Fine Chemicals and PUFAs

The term “fine chemical” is known in the art and encompasses molecules which have been produced by an organism and which are used in a variety of industries such as, by way of example but not by way of limitation, the pharmaceuticals industry, agro industry, food industry and cosmetics industry. These compounds encompass lipids, fatty acids, cofactors and enzymes and the like (as described, for example, in Kuninaka, A. (1996) Nucleotides and related compounds, pp. 561-612, in Biotechnology Vol. 6, Rehm et al., Ed., VCH Weinheim and references cited therein), lipids, saturated and unsaturated fatty acids (for example arachidonic acid), vitamins and cofactors (as described in Ullmann's Encyclopedia of Industrial Chemistry, Vol. A27, Vitamins, pp. 443-613 (1996) VCH Weinheim and references cited therein; and Ong, A. S., Niki, E., & Packer, L. (1995) Nutrition, Lipids, Health and Disease Proceedings of the UNESCO/Confederation of Scientific and Technological Associations in Malaysia and the Society for Free Radical Research—Asia, held Sep. 1-3, 1994, in Penang, Malaysia, AOCS Press (1995)), enzymes and all other chemicals described by Gutcho (1983) in Chemicals by Fermentation, Noyes Data Corporation, ISBN: 0818805086, and references cited therein. The metabolism and the uses of certain fine chemicals are illustrated in greater detail hereinbelow.

The combination of various precursor molecules and biosynthetic enzymes leads to the production of various fatty acid molecules, which has a decisive effect on membrane composition. It can be assumed that PUFAs are not only just incorporated into triacylglycerol, but also into membrane lipids.

Examples of precursors for PUFA biosynthesis are oleic acid, linoleic acid and linolenic acid. These C₁₈ carbon fatty acids must be elongated to C₂₀ and C₂₂ to give fatty acids of the eicosa and docosa chain type. Various desaturases such as enzymes which have Δ12-desaturase, Δ15-desaturase, Δ6-desaturase, Δ5- and Δ4-desaturase activity, can lead to arachidonic acid, eicosapentaenoic acid and docosahexaenoic acid and various other long-chain PUFAs which can be extracted and used for various purposes in food and feed, cosmetic or pharmaceutical applications.

To produce long-chain PUFAs, the polyunsaturated C₁₈- or C₂₀-fatty acids must be polydesaturated as mentioned above. The nucleic acid sequences according to the invention encode first functionally active desaturases from Phaeodactylum tricornutum, a microorganism comprising PUFAs in the triacylglycerol fraction. Double bonds can be introduced into the Δ5, Δ6 or Δ12 position with the desaturases according to the invention. The activities of the desaturases according to the invention preferably lead to C₁₈-+C₂₀-fatty acids with at least two, three, four or five double bonds in the fatty acid molecule, preferably to C₂₀-fatty acids with, advantageously, three, four or five double bonds in the fatty acid molecule. Desaturation can be effected before or after elongation of the fatty acid in question. The products of the desaturase activities and of the possible further desaturation and elongation therefore lead to preferred PUFAs with a higher degree of desaturation, including a further elongation of C₂₀- to C₂₂-fatty acids, to fatty acids such as linoleic acid, docosadienoic acid, dihomo-γ-linolenic acid, arachidonic acid, ω6-eicosatrienedihomo-γ-linolenic acid, eicosapentaenoic acid, ω3-eicosatrienoic acid, ω3-eicosatetraenoic acid, docosapentaenoic acid or docosahexaenoic acid. Preferred substrates of this enzyme activity according to the invention are taxoleic acid, 6,9-octadecadienoic acid, oleic acid, linoleic acid, γ-linolenic acid, pinolenic acid, α-linolenic acid, arachidonic acid, eicosapentaenoic acid or stearidonic acid. Preferred substrates are linoleic acid, γ-linolenic acid and/or α-linolenic acid, dihomo-γ-linolenic acid or arachidonic acid, eicosatetraenoic acid or eicosapentaenoic acid. The C₁₈- or C₂₀-fatty acids with at least two double bonds in the fatty acid can be elongated by the enzyme activity according to the invention in the form of the free acid or in the form of the esters, such as phospholipids, glycolipids, sphingolipids, phosphoglycerides, monoacyl glycerides, diacyl glycerides, triacyl glycerides or other esters.

Furthermore, fatty acids must subsequently be transported to various locations of modification and incorporated into the triacylglycerol storage lipid. Another important step in lipid synthesis is the transfer of fatty acids to the polar head groups, for example by glycerol fatty acid acyl transferase (see Frentzen, 1998, Lipid, 100(4-5):161-166).

For publications on plant fatty acid biosynthesis, desaturation, lipid metabolism and the membrane transport of fatty compounds, beta-oxidation, fatty acid modification and cofactors, triacylglycerol storage and assembly including the references cited therein, see the following articles: Kinney, 1997, Genetic Engeneering, Ed.: J K Setlow, 19:149-166; Ohlrogge and Browse, 1995, Plant Cell 7:957-970; Shanklin and Cahoon, 1998, Annu. Rev. Plant Physiol. Plant Mol. Biol. 49:611-641; Voelker, 1996, Genetic Engineering, Ed.: J K Setlow, 18:111-13; Gerhardt, 1992, Prog. Lipid R. 31:397-417; Gühnemann-Schäffer & Kindl, 1995, Biochim. Biophys Acta 1256:181-186; Kunau et al., 1995, Prog. Lipid Res. 34:267-342; Stymne et al., 1993, in: Biochemistry and Molecular Biology of Membrane and Storage Lipids of Plants, Ed.: Murata and Somerville, Rockville, American Society of Plant Physiologists, 150-158, Murphy & Ross 1998, Plant Journal. 13(1):1-16.

Vitamins, cofactors and nutraceuticals, such as PUFAs, encompass a group of molecules which higher animals can no longer synthesize and therefore have to take up, or which higher animals can no longer synthesize themselves to a sufficient degree and must therefore take up additionally, even though they are readily synthesized by other organisms such as bacteria. The biosynthesis of these molecules in organisms which are capable of producing them, such as in bacteria, has been largely characterized (Ullmann's Encyclopedia of Industrial Chemistry, “Vitamins”, Vol. A27, pp. 443-613, VCH Weinheim, 1996; Michal, G. (1999) Biochemical Pathways: An Atlas of Biochemistry and Molecular Biology, John Wiley & Sons; Ong, A. S., Niki, E., & Packer, L. (1995) “Nutrition, Lipids, Health and Disease” Proceedings of the UNESCO/Confederation of Scientific and Technological Associations in Malaysia and the Society for Free Radical Research Asia, held Sep. 1-3, 1994, in Penang, Malaysia, AOCS Press, Champaign, Ill. X, 374 pp.).

The abovementioned molecules are either biologically active molecules themselves or precursors of biologically active substances which act either as electron carriers or as intermediates in a multiplicity of metabolic pathways. Besides their nutritional value, these compounds also have a significant industrial value as colorants, antioxidants and catalysts or other processing auxiliaries. (For a review over structure, activity and industrial applications of these compounds, see, for example, Ullmann's Encyclopedia of Industrial Chemistry, “Vitamins”, Vol. A27, pp. 443-613, VCH Weinheim, 1996). Polyunsaturated fatty acids have a variety of functions and health-promoting effects, for example in the case of coronary heart disease, inflammatory mechanisms, children's nutrition and the like. For publications and references including the references cited therein, see: Simopoulos, 1999, Am. J. Clin. Nutr. 70 (3rd Suppl.):560-569, Takahata et al., Biosc. Biotechnol. Biochem. 1998, 62(11):2079-2085, Willich and Winther, 1995, Deutsche Medizinische Wochenschrift 120(7):229 et seq.

II. Elements and Processes of the Invention

The present invention is based, inter alia, on the discovery of novel molecules termed herein desaturase nucleic acid and desaturase protein molecules, which exert an effect on the production of cell membranes and lipids in Phaeodactylum tricornutum and, for example, have an effect on the movement of molecules via these membranes. In one embodiment, the desaturase molecules participate in the metabolism of compounds required for the synthesis of cell membranes in organisms, such as microorganisms and plants, or indirectly affect the transport of molecules via these membranes. In a preferred embodiment, the activity of the desaturase molecules according to the invention for regulating the production of membrane components and membrane transport has an effect on the production of the desired fine chemical by this organism. In an especially preferred embodiment, the activity of the desaturase molecules according to the invention is modulated so that the yield, production and/or production efficiency of the metabolic pathways of microorganisms or plants which regulate the desaturases acording to the invention are modulated and the transport efficiency of compounds through the membranes is modified, which either directly or indirectly modulates the yield, production and/or production efficiency of a desired fine chemical by microorganisms and plants.

The term “desaturase” or “desaturase polypeptide” encompasses proteins which participate in the desaturation of fatty acids. Examples of desaturases are disclosed in SEQ ID NO: 1, 3, 5, 11 or their homologues, derivatives or analogs. The terms desaturase or desaturase nucleic acid sequence(s) encompass nucleic acid sequences which encode a desaturase and part of which can be a coding region and also corresponding 5′- and 3′-untranslated sequence regions. Examples of desaturase genes are those shown in SEQ ID NO: 1, 3, 5 or 11. The terms production and productivity are known in the art and encompass the concentration of the fermentation product (for example of the desired fine chemical) which is formed within a specific period and in a specific fermentation volume (for example kg product per hour per liter). The term production efficiency encompasses the time required for achieving a particular production quantity (for example the time required by the cell to establish its particular throughput rate of a fine chemical). The term yield or product/carbon yield is known in the art and encompasses the efficiency with which the carbon source is converted into the product (i.e. the fine chemical). This is usually expressed as, for example, kg product per kg carbon source. Increasing the yield of production of the compound increases the amount of the molecules obtained or of the suitable molecules of this compound obtained in a specific quantity of culture over a defined period. The terms biosynthesis or biosynthetic pathway are known in the art and encompass the synthesis of a compound, preferably of an organic compound, by a cell from intermediates, for example in a multi-step process which is subject to strong regulation. The terms catabolism or catabolic pathway are known in the art and encompass the cleavage of a compound, preferably of an organic compound, by a cell into catabolytes (in general terms, smaller or less complex molecules), for example in a multi-step process which is subject to strong regulation. The term metabolism is known in the art and encompasses the totality of the biochemical reactions which take place in an organism. The metabolism of a certain compound (for example the metabolism of a fatty acid) thus encompasses the totality of the biosynthetic, modification and catabolic pathways of this compound in the cell which are relevant to this compound.

In another embodiment, the nucleic acid sequences according to the invention which encode desaturase molecules can modulate the production of a desired molecule, such as a fine chemical, in a microorganism or in plants. There exist a series of mechanisms by which the modification of a sequence according to the invention can directly affect the yield, production and/or production efficiency of a fine chemical from a microorganism strain or plant strain comprising this modified protein. The number or activity of desaturases participating in the transport of molecules of fine chemicals within, or out of, the cell can be increased, so that greater amounts of these compounds are transported via membranes, from which they can be obtained and converted into each other with greater ease. Furthermore, fatty acids, triacylglycerols and/or lipids are desirable fine chemicals themselves; optimizing the activity or increasing the number of one or more desaturases according to the invention which participate in the biosynthesis of these compounds, or by interfering with the activity of one or more desaturases which participate in the catabolism of these compounds, makes increasing the yield, production and/or production efficiency of fatty acid molecules and lipid molecules from organisms such as microorganisms or plants, possible.

The mutagenesis of the abovementioned nucleic acid sequences can give rise to desaturases with modified activities which indirectly affect the production of one or more desired fine chemicals from microorganisms or plants. For example, desaturases which participate in the export of waste products can exhibit a greater number or higher activity, so that the normal metabolic waste products of the cell (whose quantity might be increased owing to the overproduction of the desired fine chemical) are exported efficiently before they can damage the molecules in the cell (which would reduce cell viability) or interfere with the biosynthetic pathways of the fine chemicals (which would reduce the yield, production or production efficiency of a desired fine chemical). The relatively large intracellular amounts of the desired fine chemical themselves can furthermore be toxic to the cell, so that increasing the activity or number of transporters capable of exporting these compounds from the cell results in an increased viability of the cell in culture, which, in turn, leads to a higher number of cells in the culture which produce the desired fine chemical. The desaturases can also be manipulated in such a way that the corresponding amounts of different lipid molecules and fatty acid molecules are produced. This can have a substantial effect on the lipid concentration of the cell membrane. Since each lipid type has different physical properties, a modification of the lipid composition of a membrane can significantly modify membrane fluidity. Modifications of the membrane fluidity can affect the transport of molecules via the membrane and cell integrity, each of which has a substantial effect on the production of fine chemicals from microorganisms and plants in large-scale fermentation culture. Plant membranes impart specific properties such as tolerance to high and low temperatures, salt, drought and tolerance with respect to pathogens such as bacteria and fungi.

The abovementioned isolated nucleic acid sequences are present, for example, in the genome of a Phaeodactylum tricornutum UTEX646 strain which is available via the algae collection of the University of Texas, Austin.

The nucleotide sequence of the Phaeodactylum tricornutum cDNA and the derived amino acid sequences of the desaturases are shown in SEQ ID NO: 1 to 6 and 11 and 12. Computer analyses were carried out which classify and/or identify these nucleotide sequences as sequences which encode proteins participating in the metabolism of cell membrane components or which participate in the transport of compounds via cell membranes, or of PUFA biosynthesis. ESTs with the database input NO: PT001070010R and PT001078032R by the inventors constitute the sequences according to the invention in SEQ ID NO: 1 and 3. The sequence of the fragment of EST PT001070010R was determined and is as shown in SEQ ID NO: 5. In a similar manner, the sequence of clone PT001078032R is shown in SEQ ID NO: 1. Gene names were assigned to the clones. The abbreviations denote: Pp=Physcomitrella patens, Pt=Phaeodactylum tricornutum. PT001070010R of SEQ ID NO: 5 encodes a novel gene which is homologous to Δ12-desaturase and PT001078032R encodes a novel Δ5-desaturase. Pt_des6 can be isolated in accordance with Example 5a by means of polymerase chain reaction with the aid of degenerate oligonucleotides. A fragment obtained in this way can be isolated for screening a Phaeodactylum tricornutum cDNA library, and the coding region of a Phaeodactylum tricornutum Δ6-desaturase can be obtained. A gene isolated in this way is termed Pt_des6 in Table 1 and is shown in SEQ ID NO: 3. The corresponding amino acid sequences are obtained by translating the genetic code of sequence ID NO: 1, 3 and 5 and are defined as SEQ ID NO: 2, 4 and 6 (see also Table 1). A further nucleic acid sequence which encodes a Δ12-desaturase can also be found in Table 1. It has the clone number PT001072031R.

TABLE 1

| | Gene | | Nucleic acid | Polypeptide |
|---|---|---|---|---|
| | name | Clone name | SEQ ID NO: | SEQ ID NO: |
| Δ5-desaturase | Pt_des5 | PT001078032R | 1 | 2 |
| Δ6-desaturase | Pt_des6 | Pt_des6 | 3 | 4 |
| Δ12-desaturase | Pt_des12 | PT001070010R | 5 | 6 |
| Δ6-desaturase | Pp_des6 | Pp_des6 | 7 | 8 |
| Δ6-elongase | Pp_PSE1 | PP001019019F | 9 | 10 |
| Δ12-desaturase | Pt des12.2 | PT001072013R | 11 | 12 |

The present invention also relates to proteins with an amino acid sequence which is essentially homologous with an amino acid sequence of SEQ ID NO:2, 4, 6 or 12. As used in the present context, a protein with an amino acid sequence which is essentially homologous with a selected amino acid sequence has at least approximately 50% homology with the selected amino acid sequence, for example the complete amino acid sequence selected. A protein with an amino acid sequence which is essentially homologous with a selected amino acid sequence can also have at least approximately 50 to 60% homology, preferably at least approximately 60 to 70% homology and more preferably at least approximately 70 to 80%, 80 to 90% or 90 to 95% homology and most preferably at least approximately 96%, 97%, 98%, 99% or more homology with a selected amino acid sequence.

The desaturases or the biologically active parts or fragments thereof can participate in the metabolism of lipids required for the synthesis of membranes or storage lipids in organisms and can, in combination with further genes, in particular those with elongase activity, contribute to activities required for the elongation of C₁₈- or C₂₀₋₂₂—PUFAs so that C₁₈-, C₂₀-, C₂₂- or C₂₄-PUFAs and related PUFAs are obtained.

In this context, the desaturases can be cloned in combination with elongases and other desaturases in expression cassettes according to the invention and employed for the transformation of plants with the aid of Agrobacterium.

Various aspects of the invention are described in greater detail in the subsections which follow.

A. Isolated Nucleic Acid Molecules

One embodiment of the invention are isolated nucleic acids derived from PUFA-producing microorganisms and encoding polypeptides which desaturate C₁₈- or C₂₀₋₂₂-fatty acids with at least one, two, three or four double bonds in the fatty acid.

A further embodiment according to the invention are isolated nucleic acids encompassing nucleotide sequences encoding polypeptides which desaturate C₁₈- or C₂₀-fatty acids with at least one, two, three or four double bonds in the fatty acid and which are selected from the group consisting of

a) a nucleic acid sequence with the sequence shown in SEQ ID NO: 1, SEQ ID NO: 3, SEQ ID NO: 5 or SEQ ID NO: 11,

b) nucleic acid sequences which, owing to the degeneracy of the genetic code, are obtained by backtranslating the amino acid sequences shown in SEQ ID NO: 2, SEQ ID NO: 4, SEQ ID NO: 6 or SEQ ID NO: 12,

c) derivatives of the nucleic acid sequence shown in SEQ ID NO: 1, SEQ ID NO: 3, SEQ ID NO: 5 or SEQ ID NO: 11, which encode polypeptides with the amino acid sequences shown in SEQ ID NO: 2, SEQ ID NO: 4, SEQ ID NO: 6 or SEQ ID NO: 12 and have at least 50% homology at the amino acid level, without essentially reducing the enzymatic action of the polypeptides.

The abovementioned nucleic acid according to the invention is derived from organisms such as ciliates, fungi, algae or dinoflagellates which are capable of synthesizing PUFAs, preferably from Phaeodactylum tricornutum or closely related organisms.

One aspect of the invention relates to isolated expression cassettes and to nucleic acid molecules which encode desaturase polypeptide or biologically active parts thereof, and to nucleic acid fragments of these which suffice for use as hybridization probes or primers for identifying or amplifying a desaturase-encoding nucleic acid (for example desaturase DNA). The term “nucleic acid molecule” as used in the present context is intended to encompass DNA molecules (for example cDNA or genomic DNA) and RNA molecules (for example mRNA) and DNA or RNA analogs which are generated by means of nucleotide analogs. This term additionally encompasses the untranslated sequence on the 3′ and the 5′ ends of the coding gene region: at least 500, preferably 200, especially preferably 100, nucleotides of the sequence upstream of the 5′ end of the coding region and at least 100, preferably 50, especially preferably 20, nucleotides of the sequence downstream of the 3′ end of the coding gene region. The nucleic acid molecule can be single- or double-stranded, but is preferably double-stranded DNA. An “isolated” nucleic acid molecule is separated from other nucleic acid molecules which are present in the natural source of the nucleic acid. An “isolated” nucleic acid preferably has no sequences which naturally flank the nucleic acid in the genomic DNA of the organism from which the nucleic acid is derived (for example sequences located at the 5′ and 3′ ends of the nucleic acid). In various embodiments, the isolated desaturase nucleic acid molecule can comprise, for example, less than approximately 5 kb, 4 kb, 3 kb, 2 kb, 1 kb, 0.5 kb or 0.1 kb of nucleotide sequences which naturally flank the nucleic acid molecule in the genomic DNA of the cell from which the nucleic acid is derived (for example a Physcomitrella patens cell). An “isolated” nucleic acid molecule, such as a cDNA molecule, can moreover be essentially free from other cellular material or culture medium if it is generated by recombinant techniques, or free from chemical precursors or other chemicals if it is synthesized chemically.

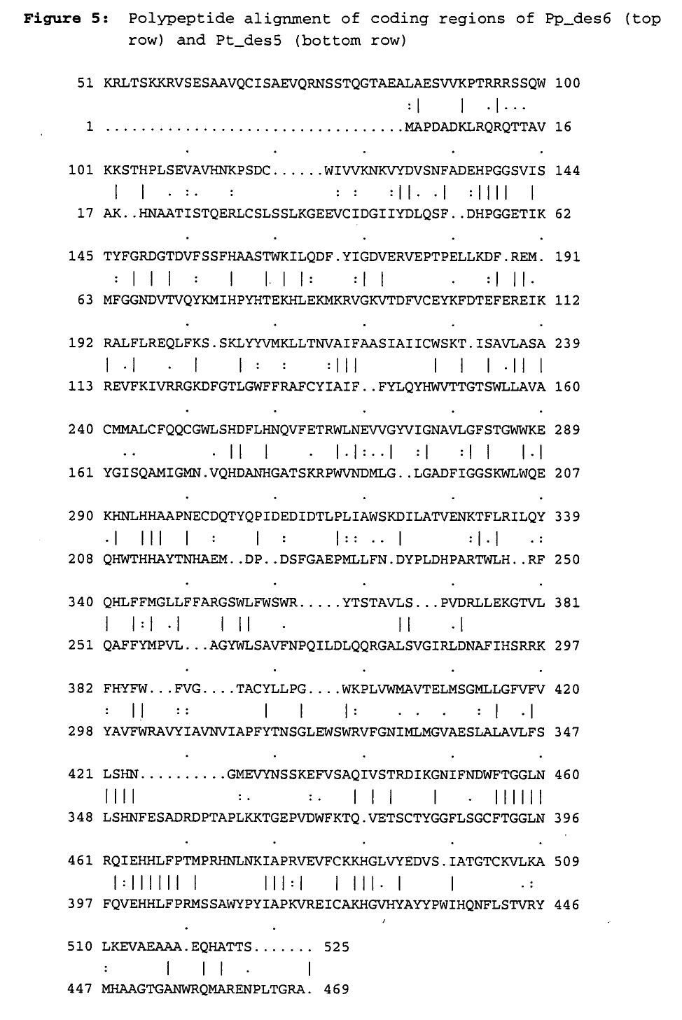

An expression cassette according to the invention having the structure SEQ ID NO: 32-promoter-SEQ ID NO: 33, SEQ ID NO: 34 or SEQ ID NO: 35 nucleic acid molecule, for example a nucleic acid molecule with a nucleotide sequence of SEQ ID NO:1 or a part thereof, can be isolated using standard techniques of molecular biology and the sequence information provided herein. Also, for example a homologous sequence or homologous, conserved sequence regions can be identified at DNA or amino acid level with the aid of alignment algorithms. For example, a Phaeodactylum tricornutum cDNA can be isolated from a Phaeodactylum tricornutum library by using the complete SEQ ID NO:1, 3, 5 or 11 or a part thereof as hybridization probe and standard hybridization techniques (as described, for example, in Sambrook et al., Molecular Cloning: A Laboratory Manual. 2nd Ed., Cold Spring Harbor Laboratory, Cold Spring Harbor Laboratory Press, Cold Spring Harbor, N.Y., 1989). Moreover, a nucleic acid molecule encompassing a complete sequence of SEQ ID NO: 1, 3, 5 or 11 or a part thereof can be isolated by polymerase chain reaction, where oligonucleotide primers which are generated on the basis of this sequence or parts thereof, in particular regions around motifs of Example 5a or modifications of the same in individual defined amino acids are used (for example, a nucleic acid molecule encompassing the complete sequence of SEQ ID NO:1, 3, 5 or 11 or a part thereof can be isolated by polymerase chain reaction using oligonucleotide primers which have been generated on the basis of this same sequence of SEQ ID NO: 1, 3, 5 or 11). For example, mRNA can be isolated from cells (for example by the guanidinium thiocyanate extraction method of Chirgwin et al. (1979) Biochemistry 18:5294-5299) and cDNA by means of reverse transcriptase (for example Moloney MLV reverse transcriptase, available from Gibco/BRL, Bethesda, Md., or AMV reverse transcriptase, available from Seikagaku America, Inc., St.Petersburg, Fla.). Synthetic oligonucleotide primers for amplification by means of polymerase chain reaction can be generated on the basis of one of the sequences shown in SEQ ID NO: 1, 3, 5 or 11 and in FIG. 5a or with the aid of the amino acid sequences shown in SEQ ID NO: 2, 4, 6 or 12. A nucleic acid according to the invention can be amplified using cDNA or, alternatively, genomic DNA as template and suitable oligonucleotide primers, in accordance with standard PCR amplification techniques. The nucleic acid amplified in this way can be cloned into a suitable vector and characterized by means of DNA sequence analysis. Oligonucleotides which correspond to a desaturase nucleotide sequence can be generated by standard synthesis methods, for example using an automatic DNA synthesizer.

The cDNA shown in SEQ ID NO: 1,3, 5 or 11 encompasses sequences which encode desaturases (i.e. the “coding region”), and 5′-untranslated sequences and 3′-untranslated sequences. Alternatively, the nucleic acid molecule can only encompass the coding region of one of the sequences in SEQ ID NO: 1, 3, 5 or 11 or can comprise complete genomic fragments which have been isolated from genomic DNA.

In a further preferred embodiment, an isolated nucleic acid molecule according to the invention encompasses a nucleic acid molecule which is a complement of one of the nucleotide sequences shown in SEQ ID NO: 1, 3, 5 or 11 or a part thereof. A nucleic acid molecule which is complementary to one of the nucleotide sequences shown in SEQ ID NO: 1, 3, 5 or 11 is sufficiently complementary if it is capable of hybridizing with one of the sequences stated in SEQ ID NO: 1, 3, 5 or 11, giving rise to a stable duplex.

Homologs of the novel desaturase nucleic acid sequences with the sequence SEQ ID NO: 1, 3, 5 or 11 means, for example, allelic variants with at least approximately 50 to 60% homology, preferably at least approximately 60 to 70% homology, more preferably at least approximately 70 to 80%, 80 to 90% or 90 to 95% homology and even more preferably at least approximately 95%, 96%, 97%, 98%, 99% or more homology with one of the nucleotide sequences shown in SEQ ID NO: 1, 3, 5 or 11 or their homologs, derivatives, analogs or parts thereof. In a further preferred embodiment, an isolated nucleic acid molecule according to the invention encompasses a nucleotide sequences which hybridizes with one of the nucleotide sequences shown in SEQ ID NO: 1, 3, 5 or 11 or a part thereof, for example under stringent conditions. Allelic variants encompass, in particular, functional variants which can be obtained by the deletion, insertion or substitution of nucleotides from/into the sequence shown in SEQ ID NO: 1, 3, 5 or 11, it being intended, however, for the enzyme activity of the resulting proteins which are synthesized to be advantageously retained for the insertion of one or more genes. Proteins which retain the enzymatic activity of desaturase, that is to say whose activity is essentially not reduced, means proteins with at least 10%, preferably 20%, especially preferably 30%, very particularly preferably 40%, of the original enzyme activity compared with the protein encoded by SEQ ID NO: 2, 4, 6 or 12.

Homologs of SEQ ID NO: 1, 3, 5 or 11 also means, for example, bacterial, fungal and plant homologs, truncated sequences, single-stranded DNA or RNA of the coding and noncoding DNA sequence.

Homologs of SEQ ID NO: 1, 3, 5 or 11 also means derivatives such as, for exmaple, promoter variants. The promoters upstream of the nucleotide sequences stated can be modified by one or more nucleotide substitutions, by insertion(s) and/or deletion(s), without, however, interfering with the functionality or activity of the promoters. It is furthermore possible for the activity of the promoters to be increased by modifying their sequence or for them to be replaced completely by more active promoters, even from heterologous organisms.

Moreover, the nucleic acid molecule according to the invention may only encompass part of the coding region of one of the sequences in SEQ ID NO: 1, 3, 5 or 11, for example a fragment which can be used as probe or primer, or a fragment which encodes a biologically active segment of a desaturase. The nucleotide sequences determined from cloning the Phaeodactylum tricornutum desaturase gene allow the generation of probes and primers which are designed for identifying and/or cloning desaturase homologs in other cell types and organisms and desaturase homologs from other microalgae or related species. The probe/primer usually encompasses an essentially purified oligonucleotide. The oligonucleotide usually encompasses a nucleotide sequence region which hybridizes under stringent conditions to at least approximately 12, preferably approximately 16, more preferably approximately 25, 40, 50 or 75 successive nucleotides of a sense strand of one of the sequences stated in SEQ ID NO: 1, 3, 5 or 11, of an antisense strand of one of the sequences stated in SEQ ID NO: 1, 3, 5 or 11 or its homologs, derivatives or analogs or naturally occurring mutants thereof. Primers based on a nucleoide sequence of SEQ ID NO: 1, 3, 5 or 11 can be used in PCR reactions for cloning desaturase homologs. Probes based on the desaturase nucleotide sequences can be used for detecting transcripts or genomic sequences which encode the same or homologous proteins. In preferred embodiments, the probe additionally encompasses a labeling group bound thereto, for example a radioisotope, a fluorescent compound, an enzyme or an enzyme cofactor. These probes can be used as part of a test kit for genomic markers for identifying cells which misexpress a desaturase, for example by measuring an amount of a desaturase-encoding nucleic acid in a cell sample, for example measuring the desaturase mRNA level, or for determining whether a genomic desaturase gene is mutated or deleted.

In one embodiment, the nucleic acid molecule according to the invention encodes a protein or part thereof which encompasses an amino acid sequence with sufficient homology with an amino acid sequence of SEQ ID NO: 2, 4, 6 or 12 for the protein or part thereof to retain the ability to participate in the metabolism of compounds required for the synthesis of the cell membranes in microorganisms or plants or in the transport of molecules via these membranes. As used in the present context, the term “sufficient homology” refers to proteins or parts thereof whose amino acid sequences have a minimum number of amino acid residues which are identical with or equivalent to an amino acid sequence of SEQ ID NO:2 (for example an amino acid residue with a similar side chain, such as an amino acid residue in one of the sequences of SEQ ID NO:2) so that the protein or the part thereof can participate in the metabolism of compounds required for the synthesis of cell membranes in microorganisms or plants or in the transport of molecules via these membranes. As described herein, protein components of these metabolic pathways for membrane components or membrane transport systems can play a role in the production and secretion of one or more fine chemicals. Examples of these activities are also described herein. Thus, the “function of a desaturase” contributes either directly or indirectly to the yield, production and/or production efficiency of one or more fine chemicals. Examples of desaturase substrate specificity of the catalytic activity are stated in Tables 5 and 6.

In a further embodiment, derivatives of the nucleic acid molecule according to the invention encode proteins with at least approximately 50 to 60% homology, preferably at least approximately 60 to 70% homology and more preferably at least approximately 70 to 80%, 80 to 90%, 90 to 95% homology, and most preferably at least approximately 96%, 97%, 98%, 99% or more homology with a complete amino acid sequence of SEQ ID NO:2. The homology of the amino acid sequence can be determined over the entire sequence region using the program PileUp (J. Mol. Evolution., 25, 351-360, 1987, Higgins et al., CABIOS, 5, 1989:151-153) or BESTFIT or GAP (Henikoff, S. and Henikoff, J. G. (1992). Amino acid substitution matrices from protein blocks. Proc. Natl. Acad. Sci. USA 89: 10915-10919.)

Parts of proteins encoded by the desaturase nucleic acid molecules according to the invention are preferably biologically active parts of one of the desaturases. As used herein, the term “biologically active part of a desaturase” is intended to encompass a segment, for example a domain/motif, of a desaturase which can participate in the metabolism of compounds required for the synthesis of cell membranes in microorganisms or plants or in the transport of molecules via these membranes or which has an activity stated in Tables 5 and 6. An assay of the enzymatic activity can be carried out in order to determine whether a desaturase or a biologically active part thereof can participate in the metabolism of compounds required for the synthesis of cell membranes in microorganisms or plants or in the transport of molecules via these membranes. These assay methods as described in detail in Example 8 of the examples section are known to the skilled worker.

Additional nucleic acid fragments which encode biologically active segments of a desaturase can be generated by isolating part of one of the sequences in SEQ ID NO: 1, 3, 5 or 11, expressing the encoded segment of the desaturase or of the peptide (for example by recombinant expression in vitro) and determining the activity of the encoded part of the desaturase or of the peptide.

Moreover, the invention encompasses nucleic acid molecules which differ from one of the nucleotide sequences shown in SEQ ID NO: 1, 3, 5 or 11 (and parts thereof) owing to the degeneracy of the genetic code and which thus encode the same desaturase as the one encoded by the nucleotide sequences shown in SEQ ID NO: 1, 3, 5 or 11. In another embodiment, an isolated nucleic acid molecule according to the invention has a nucleotide sequence which encodes a protein with an amino acid sequence shown in SEQ ID NO: 2, 4, 6 or 12. In a further embodiment, the nucleic acid molecule according to the invention encodes a full-length desaturase protein which is essentially homologous to an amino acid sequence of SEQ ID NO: 2, 4, 6 or 12 (which is encoded by an open reading frame shown in SEQ ID NO: 1, 3, 5 or 11) and which can be identified and isolated by customary methods.

In addition to the desaturase nucleotide sequence shown in SEQ ID NO: 1, 3, 5 or 11, the skilled worker recognizes that DNA sequence polymorphisms may exist which lead to changes in the amino acid sequences of the desaturases within a population (for example the Phaeodactylum tricornutum population). These genetic polymorphisms in the desaturase gene can exist between individuals within a population owing to natural variation. As used in the present context, the terms “gene” and “recombinant gene” refer to nucleic acid molecules with an open reading frame which encodes a desaturase, preferably a Phaeodactylum tricornutum desaturase. These natural variants usually cause a variance of 1 to 5% in the nucleotide sequence of the desaturase gene. All of these nucleotide variations and resulting amino acid polymorphisms in desaturase which are the result of natural variation and do not alter the functional activity of desaturases are intended to come within the scope of the invention.

Nucleic acid molecules which correspond to the natural variants and non-Phaeodactylum-tricornutum-homologs, -derivatives and analogs of the Phaeodactylum tricornutum cDNA can be isolated in accordance with standard hybridization techniques under stringent hybridization conditions owing to their homology with the Phaeodactylum tricornutum desaturase nucleic acid disclosed herein using the Phaeodactylum tricornutum cDNA or part thereof as hybridization probe. In another embodiment, an isolated nucleic acid molecule according to the invention has a minimum length of 15 nucleotides and hybridizes under stringent conditions to the nucleic acid molecule which encompasses a nucleotide sequence of SEQ ID NO:1, 3, 5 or 11. In other embodiments, the nucleic acid has a minimum length of 25, 50, 100, 250 or more nucleotides. The term “hybridizes under stringent conditions” as used in the present context is intended to describe hybridization and wash conditions under which nucleotide sequences which have at least 60% homology to each other usually remain hybridized to each other. The conditions are preferably such that sequences which have at least approximately 65% homology, more preferably approximately 70% homology and even more preferably at least approximately 75% or more homology to each other usually remain hybridized to each other. These stringent conditions are known to the skilled worker and can be found in Current Protocols in Molecular Biology, John Wiley & Sons, N.Y. (1989), 6.3.1-6.3.6. A preferred, nonlimiting example of stringent hybridization conditions are hybridizations in 6× sodium chloride/sodium citrate (=SSC) at approximately 45° C., followed by one or more wash steps in 0.2×SSC, 0.1% SDS at 50 to 65° C. It is known to the skilled worker that these hybridization conditions differ depending on the type of nucleic acid and, for example, when organic solvents are present, with regard to the temperature and the concentration of the buffer. The temperature differs, for example, under “standard hybridization conditions” depending on the type of the nucleic acid between 42° C. and 58° C. in aqueous buffer with a concentration of 0.1 to 5×SSC (pH 7.2). If organic solvent is present in the abovementioned buffer, for example 50% formamide, the temperature under standard conditions is approximately 42° C. The hybridization conditions for DNA:DNA hybrids are preferably for example 0.1×SSC and 20° C. to 45° C., preferably between 30° C. and 45° C. The hybridization conditions for DNA:RNA hybrids are preferably for example 0.1×SSC and 30° C. to 55° C., preferably between 45° C. and 55° C. The abovementioned hybridization temperatures are determined for example for a nucleic acid approximately 100 bp (=base pairs) in length and a G+C content of 50% in the absence of formamide. The skilled worker knows how the hybridization conditions required can be determined with reference to textbooks, such as the one mentioned above, or from the following textbooks: Sambrook et al., “Molecular Cloning”, Cold Spring Harbor Laboratory, 1989; Hames and Higgins (Ed.) 1985, “Nucleic Acids Hybridization: A Practical Approach”, IRL Press at Oxford University Press, Oxford; Brown (Ed.) 1991, “Essential Molecular Biology: A Practical Approach”, IRL Press at Oxford University Press.

Preferably, an isolated nucleic acid molecule according to the invention which hybridizes under stringent conditions to a sequence of SEQ ID NO:1, 3, 5 or 11 corresponds to a naturally occurring nucleic acid molecule. As used in the present context, a “naturally occurring” nucleic acid molecule refers to an RNA or DNA molecule with a nucleotide sequence which occurs in nature (for example which encodes a natural protein). In one embodiment, the nucleic acid encodes a naturally occurring Phaeodactylum tricornutum desaturase.

In addition to naturally occurring variants of the desaturase sequence which may exist in the population, the skilled worker furthermore recognizes that changes by means of mutation may also be introduced into a nucleotide sequence of SEQ ID NO: 1, 3, 5 or 11, which leads to changes in the amino acid sequence of the encoded desaturase without adversely affecting the functionality of the desaturase protein. For example, nucleotide substitutions which lead to amino acid substitutions on “nonessential” amino acid residues can be generated in a sequence of SEQ ID NO: 2, 4, 6 or 12. A “nonessential” amino acid residue is a residue which can be altered in a wild-type sequence of one of the desaturases (SEQ ID NO: 2, 4, 6 or 12) without altering, that is to say essentially reducing, the activity of the desaturase, while an “essential” amino acid residue is required for the desaturase activity. Other amino acid residues (for example those which are not conserved, or only semi-conserved, in the domain with desaturase activity), however, may not be essential for the activity and can therefore be modified without modifying the desaturase activity.

Accordingly, a further aspect of the invention relates to nucleic acid molecules which encode desaturases comprising modified amino acid residues which are not essential for the desaturase activity. These desaturases differ from a sequence in SEQ ID NO: 2, 4, 6 or 12 with regard to the amino acid sequence while still retaining at least one of the desaturase activities described herein. In one embodiment, the isolated nucleic acid molecule encompasses a nucleotide sequence encoding a protein, the protein encompassing an amino acid sequence with at least approximately 50% homology with an amino acid sequence of SEQ ID NO: 2, 4, 6 or 12 and being able to participate in the metabolism of compounds required for the synthesis of the cell membranes in Phaeodactylum tricornutum or in the transport of molecules via these membranes. The protein encoded by the nucleic acid molecule preferably has at least approximately 50 to 60% homology with one of the sequences in SEQ ID NO:2, 4, 6 or 12, more preferably at least approximately 60 to 70% homology with one of the sequences in SEQ ID NO:2, 4, 6 or 12, even more preferably at least approximately 70 to 80%, 80 to 90%, 90 to 95% homology with one of the sequences in SEQ ID NO: 2, 4, 6 or 12 and most preferably at least 96%, 97%, 98% or 99% homology with one of the sequences in SEQ ID NO: 2, 4, 6 or 12.

To determine the percentage homology of two amino acid sequences (for example one of the sequences of SEQ ID NO: 2, 4, 6 or 12 and a mutated form thereof) or of two nucleic acids, the sequences are written one underneath the other to allow optimum comparison (for example, gaps may be introduced into the sequence of a protein or of a nucleic acid in order to generate an optimal alignment with the other protein or the other nucleic acid). Then, the amino acid residues or nucleotides at the corresponding amino acid positions or nucleotide positions are compared. If a position in a sequence (for example one of the sequences of SEQ ID NO: 2, 4, 6 or 12) is occupied by the same amino acid residue or the same nucleotide as the corresponding position in the other sequence (for example a mutated form of the sequence selected from SEQ ID NO: 2, 4, 6 or 12), then the molecules are homologous at this position (i.e. amino acid or nucleic acid “homology” as used in the present context corresponds to amino acid or nucleic acid “identity”). The percentage homology between the two sequences is a function of the number of identical positions which the sequences share (i.e. % homology =number of identical positions/total number of positions×100). The terms homology and identity are thus to be considered as being synonymous.

An isolated nucleic acid molecule which encodes a desaturase which is homologous with a protein sequence of SEQ ID NO: 2, 4, 6 or 12 can be generated by introducing one or more nucleotide substitutions, additions or deletions into a nucleotide sequence of SEQ ID NO: 1, 3, 5 or 11 so that one or more amino acid substitutions, additions or deletions are introduced into the encoded protein. Mutations can be introduced into one of the sequences of SEQ ID NO: 1, 3, 5 or 11 by standard techniques, such as site-directed mutagenesis and PCR-mediated mutagenesis. Preferably, conservative amino acid substitutions are generated at one or more of the predicted nonessential amino acid residues. In a “conservative amino acid substitution”, the amino acid residue is exchanged for an amino acid residue with a similar side chain. Families of amino acid residues with similar side chains have been defined in the specialist field. These families encompass amino acids with basic side chains (for example lysine, arginine, hystidine), acidic side chains (for example aspartic acid, glutamic acid), uncharged polar side chains (for example glycine, asparagine, glutamine, serine, threonine, tyrosine, cysteine), unpolar side chains (for example alanine, valine, leucine, isoleucine, proline, phenylalanine, methionine, tryptophan), beta-branched side chains (for example threonine, valine, isoleucine) and aromatic side chains (for example tyrosine, phenylalanine, tryptophan, histidine). A predicted nonessential amino acid residue in a desaturase is thus preferably exchanged for another amino acid residue from the same side-chain family. As an alternative, in another embodiment, the mutations can be introduced randomly over all or part of the desaturase-encoding sequence, for example by saturation mutagenesis, and the resulting mutants can be screened for the desaturase activity in order to identify mutants which retain desaturase activity. Following the mutagenesis of one of the sequences of SEQ ID NO: 1, 3, 5 or 11, the encoded protein can be expressed recombinantly, and the activity of the protein can be determined, for example using the assays described herein (see examples section).

In addition to the nucleic acid molecules which encode the above-described desaturases, a further aspect of the invention relates to isolated nucleic acid molecules which are “antisense” to the nucleic acid sequences according to the invention. An “antisense” nucleic acid encompasses a nucleotide sequence which is complementary to a “sense” nucleic acid which encodes a protein, for example complementary to the coding strand of a double-stranded cDNA molecule or complementary to an mRNA sequence. Accordingly, an antisense nucleic acid can bind to a sense nucleic acid by hydrogen bonds. The antisense nucleic acid can be complementary to a complete desaturase-encoding strand or only to part thereof. In one embodiment, an antisense nucleotide acid molecule is “antisense” to a “coding region” of the coding strand of a nucleotide sequence encoding a desaturase. The term “coding region” refers to the region of the nucleotide sequence which encompasses codons which are translated into amino acid residues (for example the entire coding region which starts and ends with the stop codon, i.e. the last codon before the stop codon). In a further embodiment, the antisense nucleic acid molecule is “antisense” to a “noncoding region” of the coding strand of a nucleotide sequence encoding desaturase. The term “noncoding region” refers to 5′ and 3′ sequences which flank the coding region and are not translated into amino acids (i.e. which are also termed 5′- and 3′-untranslated regions).

Given the desaturase-encoding sequences disclosed herein of the coding strand (for example the sequences shown in SEQ ID NO: 1, 3, 5 or 11), antisense nucleic acids according to the invention can be designed in accordance with the rules of Watson-Crick base pairing. The antisense nucleic acid molecule can be complementary to all of the coding region of desaturase mRNA, but is more preferably an oligonucleotide which is “antisense” to only part of the coding or noncoding region of the desaturase mRNA. The antisense oligonucleotide can be complementary, for example, to the region around the translation start of desaturase mRNA. An antisense oligonucleotide can have a length of, for example, approximately 5, 10, 15, 20, 25, 30, 35, 40, 45 or 50 and more nucleotides. An antisense nucleic acid according to the invention can be constructed by processes known in the art using chemical synthesis and enzymatic ligation reactions. An antisense nucleic acid (for example an antisense oligonucleotide) can, for example, be synthesized chemically, making use of naturally occurring nucleotides or variously modified nucleotides which are such that they increase the biological stability of the molecules or increase the physical stability of the duplex formed between the antisense and the sense nucleic acid; for example, phosphorothioate derivatives and acridine-substituted nucleotides may be used. Examples of modified nucleotides which may be used for generating the antisense nucleic acid are, inter alia, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentyladenine, uracil-5-oxyacetic acid (v), wybutoxosin, pseudouracil, queosine, 2-thiocytosine, 5-methyl-2-thiouracil, 2-thiouracil, 4-thiouracil, 5-methyluracil, methyl uracil-5-oxyacetate, uracil-5-oxyacetic acid (v), 5-methyl-2-thiouracil, 3-(3-amino-3-N-2-carboxypropyl)uracil, (acp3)w and 2,6-diaminopurine. The antisense nucleic acid can, alternatively, be generated biologically using an expression vector into which a nucleic acid has been subcloned in antisense orientation (i.e. RNA which is transcribed by the nucleic acid introduced is in antisense orientation relative to a target nucleic acid of interest, which is described in greater detail in the subsection which follows).

The antisense nucleic acid molecules according to the invention are usually administered to a cell or generated in situ so that they hybridize with, or bind to, the cellular mRNA and/or the genomic DNA encoding a desaturase, thus inhibiting expression of the protein, for example by inhibiting transcription and/or translation. Hybridization can be effected by conventional nucleotide complementarity with the formation of a stable duplex or, for example in the case of an antisense nucleic acid molecule which binds DNA duplices, by specific interactions in the major groove of the double helix. The antisense molecule can be modified in such a manner that it specifically binds to a receptor or to an antigen expressed at the selected cell surface, for example by binding the antisense nucleic acid molecule to a peptide or an antibody, each of which binds to a cell surface receptor or an antigen. The cells can also be provided with the antisense nucleic acid molecule using the vectors described herein. Vector constructs in which the antisense nucleic acid molecule is under the control of a strong prokaryotic, viral or eukaryotic promoter, including a plant promoter, are preferred for achieving sufficient intracellular concentrations of the antisense molecules.

In a further embodiment, the antisense nucleic acid molecule according to the invention is an α-anomeric nucleic acid molecule. An α-anomeric nucleic acid molecule forms specific double-stranded hybrids with complementary RNA, the strands running parallel to each other, in contrast to ordinary β units (Gaultier et al. (1987) Nucleic Acids Res. 15:6625-6641). Moreover, the antisense nucleic acid molecule can encompass a 2′-o-methylribonucleotide (Inoue et al. (1987) Nucleic Acids Res. 15:6131-6148) or a chimeric RNA-DNA analog (Inoue et al. (1987) FEBS Lett. 215:327-330).

In a further embodiment, an antisense nucleic acid according to the invention is a ribozyme. Ribozymes are catalytic RNA molecules with ribonuclease activity which can cleave a single-stranded nucleic acid, such as an mRNA, to which they have a complementary region. Thus, ribozymes (for example hammerhead ribozymes (described in Haselhoff and Gerlach (1988) Nature 334:585-591)) can be used for the catalytic cleavage of desaturase-mRNA transcripts, in order thereby to inhibit the translation of desaturase mRNA. A ribozyme with specificity for a desaturase-encoding nucleic acid can be designed on the basis of the nucleotide sequence of one of the desaturase-cDNAs disclosed in SEQ ID NO: 1, 3, 5 or 11 (i.e. or on the basis of a heterologous sequence to be isolated in accordance with the methods taught in the present invention). For example, a derivative of a Tetrahymena-L-19-IVS RNA can be constructed in which the nucleotide sequence of the active site is complementary to the nucleotide sequence to be cleaved in a desaturase-encoding mRNA. See, for example, Cech et al., U.S. Pat. No. 4,987,071 and Cech et al., U.S. Pat. No. 5,116,742. As an alternative, desaturase mRNA can be used for selecting a catalytic RNA with a specific ribonuclease activity from among a pool of RNA molecules. See, for example, Bartel, D., and Szostak, J.W. (1993) Science 261:1411-1418.

As an alternative, desaturase gene expression can be inhibited by directing nucleotide sequences which are complementary to the regulatory region of a desaturase nucleotide sequence (for example a desaturase promoter and/or enhancer) in such a way that triple helix structures are formed which inhibit the transcription of a desaturase gene in target cells. See, in general, Helene, C. (1991) Anticancer Drug Res. 6(6) 569-84; Helene, C., et al. (1992) Ann. N.Y. Acad. Sci. 660:27-36; and Maher. L.J. (1992) Bioassays 14(12):807-815.

B. Gene Construct (=Nucleic Acid Construct, Nucleic Acid Fragment or Expression Cassette)

The expression cassette according to the invention is to be understood as meaning the sequences mentioned in SEQ ID NO: 1, SEQ ID NO: 3, SEQ ID NO: 5 or SEQ ID NO: 11 which are the result of the genetic code, and/or their functional or nonfunctional derivatives, which were advantageously linked functionally to one or more regulatory signals for increasing gene expression and which advantageously control the expression of the coding sequence in the host cell. These regulatory sequences are intended to make possible the targeted expression of the genes and the protein expression. Depending on the host organism, this may mean, for example, that the gene is expressed and/or overexpressed only after induction or else that it is expressed and/or overexpressed immediately. For example, these regulatory sequences take the form of sequences to which inductors or repressors bind, thus regulating the expression of the nucleic acid. In addition to these novel regulatory sequences, or instead of these sequences, the natural regulation of these sequences may still be present before the actual structural genes and, if appropriate, may have been modified genetically, so that natural regulation was eliminated and the expression of the genes increased. However, the gene construct may also have a simpler structure, that is to say no additional regulatory signals have been inserted before the nucleic acid sequence or its derivatives, and the natural promoter together with its regulation has not been removed. Instead, the natural regulatory sequence has been mutated in such a way that regulation no longer takes place and/or gene expression is increased. These modified promoters may also be arranged by themselves in the form of part-sequences (=promoter with parts of the nucleic acid sequences according to the invention) before the natural gene in order to increase the activity. Moreover, the gene construct may advantageously also comprise one or more of what are known as enhancer sequences linked functionally to the promoter, and these make possible an increased expression of the nucleic acid. It is also possible to insert additional advantageous sequences on the 3′ end of the DNA sequences, such as further regulatory elements or terminators. The Δ5-desaturase/Δ6-desaturase and/or Δ12-desaturase genes may be present in one or more copies in the expression cassette (=gene construct).

In this context, the regulatory sequences or factors can preferably have a positive effect on, and thus increase, the expression of the genes introduced, as has been described above. An enhancement of the regulatory elements can advantageously take place at the transcriptional level by using strong transcription signals such as promoters and/or enhancers. In addition, however, translation may also be enhanced, for example by increasing the stability of the mRNA.

A further embodiment of the invention comprises one or more gene constructs comprising one or more sequences which are defined by SEQ ID NO: 1, 3, 5, 7, 9 or 11 and which encode polypeptides in accordance with SEQ ID NO: 2, 4, 6, 8, 10 or 12. SEQ ID NO: 1, 3, 5, 7 and 11 are derived from desaturases, while SEQ ID NO: 9 encodes an elongase. Desaturases encode enzymes which introduce a double bond at the Δ5, Δ6 or Δ12 position, the substrate having one, two, three or four double bonds. The sequence shown in SEQ ID NO: 9 encodes an enzyme activity which elongates a fatty acid by at least two carbon atoms, and the homologs, derivatives or analogs which are linked functionally to one or more regulatory signals, advantageously for increasing gene expression. Examples of these regulatory sequences are sequences to which inductors or repressors bind, thus regulating the expression of the nucleic acid. In addition to these novel regulatory sequences, the natural regulation of these sequences may still be present before the actual structural genes and, if appropriate, can have been genetically modified so that the natural regulation has been eliminated and the expression of the genes has been increased. However, the gene construct may also have a simpler structure, that is to say no additional regulatory signals have been inserted before the sequence SEQ ID NO: 1, 3, 5 or 11 or their homologs and the natural promoter with its regulation has not been deleted. Instead, the natural regulatory sequence has been mutated in such a way that regulation no longer takes place and gene expression is enhanced. The gene construct may furthermore advantageously encompass one or more of what are known as enhancer sequences which are linked functionally to the promoter and which make possible increased expression of the nucleic acid sequence. It is also possible additionally to insert advantageous sequences at the 3′ end of the DNA sequences, for example further regulatory elements or terminators. The desaturase genes and the elongase gene may be present in one or more copies in the gene construct. They may be present in one gene construct or more than one gene construct. This gene construct or the gene constructs can be expressed together in the host organism. In this context, the gene construct or the gene constructs can be inserted into one or more vectors and be present in the cell in free form or else inserted into the genome. It is advantageous for the insertion of further genes into organisms if further genes are present in the gene construct.

Advantageous regulatory sequences for the novel process exist, for example, in promoters such as the cos, tac, trp, tet, trp-tet, lpp, lac, lpp-lac, lacI^{p-}, T7, T5, T3, gal, trc, ara, SP6, λ-P_{R} or λ-P_{L} promoter and are advantageously used in Gram-negative bacteria. Further advantageous regulatory sequences exist, for example, in the Gram-positive promoters amy and SPO2, in the yeast or fungal promoters ADC1, MFα, AC, P-60, CYC1, GAPDH, TEF, rp28, ADH or in the plant promoters CaMV 35S [Franck et al., Cell 21 (1980) 285-294], PRP1 [Ward et al., Plant. Mol. Biol. 22 (1993)], SSU, OCS, lib4, usp, STLS1, B33, nos or in the ubiquitin or phaseolin promoter. Advantageous in this context are also inducible promoters, such as the promoters described in EP-A-0 388 186 (benzylsulfonamide-inducible), Plant J. 2, 1992:397-404 (Gatz et al., tetracyclin-inducible), EP-A-0 335 528 (abscisic-acid-inducible) or WO 93/21334 (ethanol- or cyclohexenol-inducible). Further suitable plant promoters are the promoter of cytosolic FBPase or the potato ST-LSI promoter (Stockhaus et al., EMBO J. 8, 1989, 2445), the Glycine max phosphoribosylpyrophosphate amidotransferase promoter (Genbank Accession No. U87999) or the node-specific promoter described in EP-A-0 249 676. Especially advantageous promoters are those which allow expression in tissues which are involved in fatty acid biosynthesis. Very especially advantageous are seed-specific promoters such as the USP promoter in accordance with the embodiment, and also other promoters such as the LEB4 (Baeumlein et al., Plant J., 1992, 2 (2):233-239), DC3 (Thomas, Plant Cell 1996, 263:359-368), the phaseolin or the napin promotor. Further especially advantageous promoters are seed-specific promoters which can be used for monocots or dicots which are described in U.S. Pat. No. 5,608,152 (oilseed rape napin promoter), WO 98/45461 (Arabidopsis oleosin promoter), U.S. Pat. No. 5,504,200 (Phaseolus vulgaris phaseolin promoter), WO 91/13980 (Brassica Bce4 promoter), by Baeumlein et al., Plant J., 1992, 2 (2):233-239 (LeB4 promoter from a legume), these promoters being suitable for dicots. The following promoters are suitable, for example, for monocots: the barley 1pt-2 or 1pt-1 promoter (WO 95/15389 and WO 95/23230), the barley Hordein promoter, and other suitable promoters described in WO 99/16890.

In principle, it is possible to use all natural promoters with their regulatory sequences, such as those mentioned above, for the novel process. It is also possible and advantageous additionally to use synthetic promoters.

As described above, the gene construct can also encompass further genes which are to be introduced into the organisms. It is possible and advantageous to introduce into the host organisms, and to express therein, regulatory genes such as genes for inductors, repressors or enzymes which, owing to the enzymatic activity, engage in the regulation of one or more genes of a biosynthetic pathway. These genes can be of heterologous or homologous origin. Moreover, the nucleic acid construct or gene construct may advantageously comprise further biosynthesis genes of the fatty acid or lipid metabolism or else these genes may be present on a further, or several further, nucleic acid constructs. A biosynthesis gene of the fatty acid or lipid metabolism which is advantageously selected is a gene from the group consisting of acyl-CoA dehydrogenase(s), acyl-ACP[=acyl carrier protein] desaturase(s), acyl-ACP thioesterase(s), fatty acid acyltransferase(s), fatty acid synthase(s), fatty acid hydroxylase(s), acetyl-coenzyme A carboxylase(s), acyl-coenzyme, A-oxidase(s), fatty acid desaturase(s), fatty acid acetylenases, lipoxygenases, triacylglycerol lipases, allenoxide synthases, hydroperoxide lyases or fatty acid elongase(s) or their combinations.

For expressing the other genes which are present, gene constructs advantageously encompass further 3′- and/or 5′-terminal regulatory sequences for enhancing expression, and these are selected for optimal expression as a function of the host organism chosen and the gene(s). These regulatory sequences, as mentioned above, are intended to make possible the specific expression of the genes and protein expression. Depending on the host organism, this may mean, for example, that the gene is expressed or overexpressed only after induction, or that it is expressed and/or overexpressed immediately.

Moreover, the regulatory sequences or regulatory factors can preferably have an advantageous effect on the expression of the genes which have been introduced, thus enhancing them. In this manner, it is possible that the regulatory elements are advantageously enhanced at the transcriptional level, using strong transcription signals such as promoters and/or enhancers. However, it is furthermore also possible to enhance translation, for example by improving mRNA stability.

C. Recombinant Expression Vectors and Host Cells

A further aspect of the invention relates to vectors, preferably expression vectors, comprising a nucleic acid encoding a desaturase alone (or a part thereof) or a nucleic acid construct described under item B in which the nucleic acid according to the invention is present alone or in combination with further biosynthesis genes of the fatty acid or lipid metabolism, such as desaturases or elongases. As used in the present context, the term “vector” refers to a nucleic acid molecule which can transport another nucleic acid to which it is bound. One type of vector is a “plasmid”, which represents a circular double-stranded DNA loop into which additional DNA segments can be ligated. A further type of vector is a viral vector, it being possible for additional DNA segments to be ligated into the viral genome. Certain vectors are capable of autonomous replication in a host cell into which they have been introduced (for example bacterial vectors with a bacterial origin of replication, and episomal mammalian vectors). Other vectors (for example nonepisomal mammalian vectors) are integrated into the genome of a host cell upon introduction into the host cell and are thus replicated together with the host genome. In addition, certain vectors can govern the expression of genes to which they are linked functionally. These vectors are referred to as “expression vectors” herein. Usually, expression vectors which are suitable for recombinant DNA techniques can take the form of plasmids. In the present description, “plasmid” and “vector” may be used interchangeably since the plasmid is the most frequently used form of vector. However, the invention is intended to encompass these other forms of expression vectors, such as viral vectors (for example replication-deficient retroviruses, adenoviruses and adeno-related viruses) which exert similar functions. Furthermore, the term vector is also intended to encompass other vectors known to the skilled worker, such as phages, viruses such as SV40, CMV, baculovirus, adenovirus, transposons, IS elements, phasmids, phagemids, cosmids, linear or circular DNA.

The recombinant expression vectors according to the invention encompass a nucleic acid according to the invention or a gene construct according to the invention in a form which is suitable for expressing the nucleic acid in a host cell, which means that the recombinant expression vectors encompass one or more regulatory sequences, selected on the basis of the host cells to be used for expression, which is/are linked functionally to the nucleic acid sequence to be expressed. In a recombinant expression vector “linked functionally” means that the nucleotide sequence of interest is bound to the regulatory sequence(s) in such a way that expression of the nucleotide sequence is possible and that they are bound to each other so that both sequences fulfil the predicted function which has been ascribed to the sequence (for example in an in-vitro transcription/translation system or in a host cell, when the vector is introduced into the host cell). The term “regulatory sequence” is intended to encompass promoters, enhancers and other expression control elements (for example polyadenylation signals). These regulatory sequences are described, for example, in Goeddel: Gene Expression Technology: Methods in Enzymology 185, Academic Press, San Diego, Calif. (1990), or see: Gruber and Crosby, in: Methods in Plant Molecular Biology and Biotechnolgy, CRC Press, Boca Raton, Fla., Ed.: Glick and Thompson, Chapter 7, 89-108, including the references therein. Regulatory sequences encompass those which control the constitutive expression of a nucleotide sequence in many types of host cell and those which control the direct expression of the nucleotide sequence only in certain host cells under certain conditions. The skilled worker knows that the design of the expression vector may depend on factors such as the choice of the host cell to be transformed, the extent to which the desired protein is expressed, and the like. The expression vectors according to the invention can be introduced into host cells in order to produce proteins or peptides, including fusion proteins or fusion peptides, which are encoded by the nucleic acids as described herein (for example desaturases, mutant forms of desaturases, fusion proteins and the like).

The recombinant expression vectors according to the invention can be designed for expressing desaturases and elongases in prokaryotic and eukaryotic cells. For example, desaturase genes can be expressed in bacterial cells, such as C. glutamicum, insect cells (using baculovirus expression vectors), yeast and other fungal cells (see Romanos, M. A., et al. (1992) “Foreign gene expression in yeast: a review”, Yeast 8:423-488; van den Hondel, C. A. M. J. J., et al. (1991) “Heterologous gene expression in filamentous fungi”, in: More Gene Manipulations in Fungi, J. W. Bennet & L. L. Lasure, Ed., pp. 396-428: Academic Press: San Diego; and van den Hondel, C. A. M. J. J., & Punt, P. J. (1991) “Gene transfer systems and vector development for filamentous fungi”, in: Applied Molecular Genetics of Fungi, Peberdy, J. F., et al., Ed., pp. 1-28, Cambridge University Press: Cambridge), algae (Falciatore et al., 1999, Marine Biotechnology.1, 3:239-251), ciliates of the following types: Holotrichia, Peritrichia, Spirotrichia, Suctoria, Tetrahymena, Paramecium, Colpidium, Glaucoma, Platyophrya, Potomacus, Pseudocohnilembus, Euplotes, Engelmaniella and Stylonychia, in particular the species Stylonychia lemnae, using vectors and following a transformation method as described in WO 98/01572, and cells of multicelled plants (see Schmidt, R. and Willmitzer, L. (1988) “High efficiency Agrobacterium tumefaciens-mediated transformation of Arabidopsis thaliana leaf and cotyledon explants” Plant Cell Rep.:583-586; Plant Molecular Biology and Biotechnology, C Press, Boca Raton, Fla., Chapter 6/7, pp. 71-119 (1993); F. F. White, B. Jenes et al., Techniques for Gene Transfer, in: Transgenic Plants, Vol. 1, Engineering and Utilization, Ed.: Kung and R. Wu, Academic Press (1993), 128-43; Potrykus, Annu. Rev. Plant Physiol. Plant Molec. Biol. 42 (1991), 205-225 (and references cited therein)) or mammalian cells. Suitable host cells are furthermore discussed in Goeddel, Gene Expression Technology: Methods in Enzymology 185, Academic Press, San Diego, Calif. (1990). As an alternative, the recombinant expression vector can be transcribed and translated in vitro, for example using T7 promoter regulatory sequences and T7 polymerase.

In prokaryotes, proteins are usually expressed with vectors containing constitutive or inducible promoters which control the expression of fusion proteins or nonfusion proteins. Fusion vectors add a series of amino acids to a protein encoded therein, usually on the amino terminus of the recombinant protein, but also on the C terminus or fused within suitable regions in the proteins. These fusion vectors usually have three tasks: 1) to enhance the expression of recombinant protein; 2) to increase the solubility of the recombinant protein and 3) to support the purification of the recombinant protein by acting as ligand in affinity purification. In the case of fusion expression vectors, a proteolytic cleavage site is frequently introduced at the site where the fusion moiety and the recombinant protein are linked, so that the recombinant protein can be separated from the fusion unit after purification of the fusion protein. These enzymes and their corresponding recognition sequences encompass factor Xa, thrombin and enterokinase.

Typical fusion expression vectors are, inter alia, pGEX (Pharmacia Biotech Inc; Smith, D. B., and Johnson, K. S. (1988) Gene 67:31-40), PMAL (New England Biolabs, Beverly, Mass.) and pRIT5 (Pharmacia, Piscataway, N.J.), where glutathione S-transferase (GST), maltose-E-binding protein or protein A is fused to the recombinant target protein. In one embodiment, the desaturase-encoding sequence is cloned into a pGEX expression vector to generate a vector encoding a fusion protein which encompasses, from the N terminus to the C terminus, GST-thrombin cleavage site-X-protein. The fusion protein can be purified by affinity chromatography using glutathione-agarose resin. Recombinant desaturase which is not fused to GST can be obtained by cleaving the fusion protein with thrombin.

Examples of suitable inducible non-fusion E. coli expression vectors are, inter alia, pTrc (Amann et al. (1988) Gene 69:301-315) and pET lld (Studier et al., Gene Expression Technology: Methods in Enzymology 185, Academic Press, San Diego, Calif. (1990) 60-89). Target gene expression of the pTrc vector is based on the transcription by host RNA polymerase from a hybrid trp-lac fusion promoter. Target gene expression from the pET 11d vector is based on transcription from a T7-gn10-lac fusion promoter which is mediated by a coexpressed viral RNA polymerase (T7 gn1). This viral polymerase is provided by the host strains BL21 (DE3) or HMS174 (DE3) by a resident λ prophage which harbors a T7 gn1 gene under the transcriptional control of the lacUV 5 promoter.

Other vectors which are suitable for use in prokaryotic organisms are known to the skilled worker; these vectors are, for example, in E. coli pLG338, pACYC184, the pBR series such as pBR322, the pUC series such as pUC18 or pUC19, the M113 mp series, pKC30, pRep4, pHS1, pHS2, pPLc236, pMBL24, pLG200, pUR290, pIN-III¹¹³-B1, λt11 or pBdCI, in Streptomyces pIJ101, pIJ364, pIJ702 or pIJ361, in Bacillus pUB110, pC194 or pBD214, in Corynebacterium pSΔ77 or pAJ667. A strategy of maximizing the expression of recombinant protein is to express the protein in a host bacterium whose ability to cleave the recombinant protein proteolytically is disrupted (Gottesman, S., Gene Expression Technology: Methods in Enzymology 185, Academic Press, San Diego, Calif. (1990) 119-128). A further strategy is to modify the nucleic acid sequence of the nucleic acid to be inserted into an expression vector, so that the individual codons for each amino acid are those which are preferentially used in a bacterium selected for expression, such as C. glutamicum, et al. (Wada et al. (1992) Nucleic Acids Res. 20:2111-2118). Modification of these nucleic acid sequences according to the invention is carried out by standard techniques of DNA synthesis.

In a further embodiment, the desaturase expression vector is a yeast expression vector. Examples of vectors for expression in the yeast S. cerevisiae include pYeDesaturasec1 (Baldari et al. (1987) Embo J. 6:229-234), pMFa (Kurjan and Herskowitz (1982) Cell 30:933-943), pJRY88 (Schultz et al. (1987) Gene 54:113-123) and pYES2 (Invitrogen Corporation, San Diego, Calif.). Vectors and methods for the construction of vectors which are suitable for use in other fungi, such as the filamentous fungi, include those which are described in detail in: van den Hondel, C. A. M. J. J., & Punt, P. J. (1991) “Gene transfer systems and vector development for filamentous fungi”, in: Applied Molecular Genetics of fungi, J. F. Peberdy et al., Ed., pp. 1-28, Cambridge University Press: Cambridge, or in: More Gene Manipulations in Fungi [J. W. Bennet & L. L. Lasure, Ed., pp. 396-428: Academic Press: San Diego]. Further suitable yeast vectors are, for example, pAG-1, YEp6, YEp13 or pEMBLYe23.

As an alternative, the desaturases according to the invention can be expressed in insect cells using baculovirus expression vectors. Baculovirus vectors which are available for expressing proteins in cultured insect cells (for example Sf9 cells) include the pAc series (Smith et al. (1983) Mol. Cell Biol. 3:2156-2165) and the pVL series (Lucklow and Summers (1989) Virology 170:31-39).

The abovementioned vectors are just a short review of possible suitable vectors. Further plasmids are known to the skilled worker and are described, for example, in: Cloning Vectors (Ed. Pouwels, P. H., et al., Elsevier, Amsterdam-N.Y.- Oxford, 1985, ISBN 0 444 904018).

In yet a further embodiment, a nucleic acid according to the invention is expressed in mammalian cells using a mammalian expression vector. Mammals for the purposes of the invention are to be understood as all non-human mammals. Examples of mammalian expression vectors include pCDM8 (Seed, B. (1987) Nature 329:840) and pMT2PC (Kaufman et al. (1987) EMBO J. 6:187-195). When used in mammalian cells, the control functions of the expression vector are frequently provided by viral regulatory elements. Promoters which are usually used are derived, for example, from polyoma, adenovirus2, cytomegalovirus and Simian Virus 40. Other suitable expression systems for prokaryotic and eukaryotic cells can be found in Chapters 16 and 17 of Sambrook, J., Fritsch, E. F., and Maniatis, T., Molecular Cloning: A Laboratory Manual, 2nd Edition, Cold Spring Harbor Laboratory, Cold Spring Harbor Laboratory Press, Cold Spring Harbor, N.Y., 1989.

In another embodiment, the recombinant mammalian expression vector can control the expression of the nucleic acid preferably in a specific cell type (for example, tissue-specific regulatory elements are used for expressing the nucleic acid). Tissue-specific regulatory elements are known in the art. Nonlimiting examples of suitable tissue-specific promoters are, inter alia, the albumen promoter (liver-specific; Pinkert et al. (1987) Genes Dev. 1:268-277), lymphoid-specific promoters (Calame and Eaton (1988) Adv. Immunol. 43:235-275), in particular promoters of T-cell receptors (Winoto and Baltimore (1989) EMBO J. 8:729-733) and immunglobulins (Banerji et al. (1983) Cell 33:729-740; Queen and Baltimore (1983) Cell 33:741-748), neuron-specific promoters (for example neurofilament promoter; Byrne and Ruddle (1989) PNAS 86:5473-5477), pancreas-specific promoters (Edlund et al., (1985) Science 230:912-916) and mamma-specific promoters (for example milk serum promoter; U.S. Pat. No. 4,873,316 and European Patent Application document No. 264,166). Also included are development-regulated promoters, for example the mouse hox promoters (Kessel and Gruss (1990) Science 249:374-379) and the fetoprotein promoter (Campes and Tilghman (1989) Genes Dev. 3:537-546).

In a further embodiment, the desaturases according to the invention can be expressed in single-celled plant cells (such as algae), see Falciatore et al., 1999, Marine Biotechnology 1 (3):239-251 and references cited therein, and plant cells from higher plants (for example spermatophytes such as crops). Examples of plant expression vectors include those which are described in detail in: Becker, D., Kemper, E., Schell, J., and Masterson, R. (1992) “New plant binary vectors with selectable markers located proximal to the left border”, Plant Mol. Biol. 20:1195-1197; and Bevan, M. W. (1984) “Binary Agrobacterium vectors for plant transformation”, Nucl. Acids Res. 12:8711-8721; Vectors for Gene Transfer in Higher Plants; in: Transgenic Plants, Vol. 1, Engineering and Utilization, Ed.: Kung and R. Wu, Academic Press, 1993, pp. 15-38.

A plant expression cassette preferably comprises regulatory sequences which can control gene expression in plant cells and which are linked functionally so that each sequence can fulfil its function, such as transcriptional termination, for example polyadenylation signals. Preferred polyadenylation signals are those derived from Agrobacterium tumefaciens T-DNA, such as gene 3 of the Ti plasmid pTiACH5, which is known as octopine synthase (Gielen et al., EMBO J. 3 (1984) 835 et seq.) or functional equivalents thereof, but all other terminators which are functionally active in plants are also suitable.

Since plant gene expression is very frequently not limited to the transcription level, a plant expression cassette preferably comprises other functionally linked sequences, such as translation enhancers, for example the overdrive sequence, which contains the 5′-untranslated tobacco mosaic virus leader sequence, which increases the protein/RNA ratio (Gallie et al., 1987, Nucl. Acids Research 15:8693-8711).

Plant gene expression must be linked functionally to a suitable promoter which effects gene expression in a cell- or tissue-specific manner with the correct timing. Preferred promoters are those which lead to constitutive expression (Benfey et al., EMBO J. 8 (1989) 2195-2202), such as those which are derived from plant viruses such as ³⁵S CAMV (Franck et al., Cell 21 (1980) 285-294), 19S CaMV (see also U.S. Pat. No. 5,352,605 and WO 84/02913) or plant promoters such as the Rubisco small subunit promoter described in U.S. Pat. No. 4,962,028.

Other sequences which are preferred for use for functional linkage in plant gene expression cassettes are targeting sequences which are required for targeting the gene product into its correspoinding cell compartment (for a review, see Kermode, Crit. Rev. Plant Sci. 15, 4 (1996) 285-423 and references cited therein), for example into the vacuole, the nucleus, all types of plastids such as amyloplasts, chloroplasts, chromoplasts, the extracellular space, the mitochondria, the endoplasmic reticulum, elaioplasts, peroxisomes and other compartments of plant cells.

Plant gene expression can also be facilitated via a chemically inducible promoter (for a review, see Gatz 1997, Annu. Rev. Plant Physiol. Plant Mol. Biol., 48:89-108). Chemically inducible promoters are particularly suitable when it is desired for gene expression to take place in a specific manner with regard to timing. Examples of such promoters are a salicylic acid-inducible promoter (WO 95/19443), a tetracyclin-inducible promoter (Gatz et al. (1992) Plant J. 2, 397-404) and an ethanol-inducible promoter.

Other suitable promoters are promoters which respond to biotic or abiotic stress conditions, for example the pathogen-induced PRP1 gene promoter (Ward et al., Plant. Mol. Biol. 22 (1993) 361-366), the heat-inducible tomato hsp80 promoter (U.S. Pat. No. 5,187,267), the low temperature-inducible potato alpha-amylase promoter (WO 96/12814) or the wound-inducible pinII promoter (EP-A-0 375 091).

Promoters which are particularly preferred are those which lead to gene expression in tissues and organs in which lipid and oil biosynthesis take place, in seed cells such as endosperm cells and cells of the developing embryo. Promoters which are suitable are the oilseed rape napin gene promoter (U.S. Pat. No. 5,608,152), the Vicia faba USP promoter (Baeumlein et al., Mol Gen Genet, 1991, 225 (3):459-67), the Arabidopsis oleosin promoter (WO 98/45461), the Phaseolus vulgaris phaseolin promoter (U.S. Pat. No. 5,504,200), the Brassica Bce4 promoter (WO 91/13980) or the legumin B4 promoter (LeB4; Baeumlein et al., 1992, Plant Journal, 2 (2):233-9), and promoters which lead to the seed-specific expression in monocots such as maize, barley, wheat, rye, rice and the like. Notable promoters which are suitable are the barley 1pt2 or 1pt1 gene promoter (WO 95/15389 and WO 95/23230) or the promoters described in WO 99/16890 (promoters from the barley hordein gene, the rice glutelin gene, the rice oryzin gene, the rice prolamin gene, the wheat gliadin gene, the wheat glutelin gene, the maize zein gene, the oat glutelin gene, the sorghum kasirin gene, the rye secalin gene).

The multiparallel expression of desaturases according to the invention, alone or in combination with other desaturases or elongases, may be desired in particular. The introduction of such expression cassettes can be effected by a simultaneous transformation of a plurality of individual expression constructs or by combining a plurality of expression cassettes on one construct. Also, a plurality of vectors can be transformed with in each case a plurality of expression cassettes, and transferred to the host cell.

Promoters which are also particularly suitable are those which lead to plastid-specific expression, since plastids are the compartment in which the precursors and some end products of lipid biosynthesis are synthesized. Suitable promoters such as the viral RNA polymerase promoter are described in WO 95/16783 and WO 97/06250, and the Arabidopsis clpP promoter, described in WO 99/46394.

The invention furthermore provides a recombinant expression vector encompassing a DNA molecule according to the invention which is cloned into the expression vector in antisense orientation, i.e. the DNA molecule is linked functionally to a regulatory sequence in such a way that it allows the expression (by transcribing the DNA molecule) of an RNA molecule which is “antisense” to the desaturase mRNA. Regulatory sequences may be selected which are linked functionally to a nucleic acid cloned in antisense orientation and which control the continuous expression of the antisense RNA molecule in a multiplicity of cell types, for example, viral promoters and/or enhancers or regulatory sequences may be selected which control the constitutive, tissue-specific or cell-type-specific expression of antisense RNA. The antisense expression vector may be present in the form of a recombinant plasmid, phagemid or attenuated virus in which the antisense nucleic acids are produced under the control of a highly effective regulatory region whose activity can be determined by the cell type into which the vector has been introduced. For an explanation of the regulation of gene expression by means of antisense genes, see Weintraub, H., et al., Antisense-RNA as a molecular tool for genetic analysis, Reviews—Trends in Genetics, Vol. 1(1) 1986.

A further aspect of the invention relates to host cells into which a recombinant expression vector according to the invention has been introduced. The terms “host cell” and “recombinant host cell” are used interchangeably in the present context. Naturally, these terms do not only refer to the particular target cell, but also to the progeny or potential progeny of this cell. Since specific modifications may occur in subsequent generations owing to mutation or environmental effects, this progeny is not necessarily identical with the parental cell, but remains within the scope of the term as used in the present context.

The terms recombinant or transgene, for example recombinant expression vector or recombinant host or host cells is to be understood as meaning, for the purpose of the invention, that the nucleic acids according to the invention and/or their natural regulatory sequences at the 5′ and 3′ positions of the nucleic acids are not in their natural environment, that is to say either the location of the sequences in the original organism was altered or the nucleic acid sequences and/or the regulatory sequences were mutated in it or the nucleic acid sequences according to the invention were transferred into an organism other than the original organism or their regulatory sequences. Combinations of these modifications are also possible. Natural environment is to be understood as meaning the location of a nucleic acid sequence in an organism as it occurs in nature.

A host cell may be a prokaryotic or eukaryotic cell. For example a desaturase can be expressed in bacterial cells such as C. glutamicum, insect cells, fungal cells or mammalian cells (such as Chinese hamster ovary cells (CHO) or COS cells), algae, ciliates, plant cells, fungi or other microorganisms such as C. glutamicum. Other suitable host cells are known to the skilled worker.

Vector DNA can be introduced into prokaryotic or eukaryotic cells by conventional transformation or transfection techniques. The terms “transformation” and “transfection”, conjugation and transduction as used in the present context are intended to encompass a multiplicity of methods known in the art for introducing foreign nucleic acid (for example DNA) into a host cell, including calcium phosphate or calcium chloride coprecipitation, DEAE-dextran-mediated transfection, lipofection, natural competence, chemically mediated transfer, electroporation or particle bombardment. Suitable methods for the transformation or transfection of host cells, including plant cells, can be found in Sambrook et al. (Molecular Cloning: A Laboratory Manual., 2nd Ed., Cold Spring Harbor Laboratory, Cold Spring Harbor Laboratory Press, Cold Spring Harbor, N.Y., 1989) and other laboratory text books, such as Methods in Molecular Biology, 1995, Vol. 44, Agrobacterium protocols, Ed.: Gartland and Davey, Humana Press, Totowa, N.J.

It is known about the stable transfection of mammalian cells that only a small number of the cells integrate the foreign DNA into their genome, depending on the expression vector used and the transfection technique used. To identify and select these integrants, a gene which encodes a selectable marker (for example resistance to antibiotics) is usually introduced into the host cells together with the gene of interest. Preferred selectable markers encompass those which impart resistance to drugs such as G418, hygromycin and methotrexate, or, in plants, those which impart resistance to a herbicide such as glyphosate or glufosinate. Further suitable markers are, for example, markers which encode genes which are involved in the biosynthetic pathways of, for example, sugars or amino acids, such as β-galactosidase, ura3 or ilv2. Markers which encode genes such as luciferase, gfp or other fluorescence genes are also suitable. These markers can be used in mutants in which these genes are not functional since they have been deleted for example by means of conventional methods. Furthermore, markers which encode a nucleic acid which encodes a selectable marker can be introduced into a host cell on the same vector as the one which encodes a desaturase, or can be introduced on a separate vector. Cells which have been transfected stably with the nucleic acid introduced can be identified for example by drug selection (for example, cells which have the selectable marker integrated survive, whereas the other cells die).

To generate a microorganism with homologous recombination, a vector is generated which contains at least one segment of a desaturase gene into which a deletion, addition or substitution has been introduced in order to modify the desaturase gene hereby, for example to functionally disrupt it. This desaturase gene is preferably a Phaeodactylum tricornutum desaturase gene, but a homolog or analog from other organisms, even from mammalian, fungal or insect cells, can also be used. In a preferred embodiment, the vector is designed in such a way that the endogenous desaturase gene is functionally disrupted (i.e. no longer encodes a functional protein, also termed knock-out vector) upon homologous recombination. As an alternative, the vector can be designed in such a way that the endogenous desaturase gene is mutated or modified otherwise upon homologous recombination while still encoding a functional protein (for example, the upstream regulatory region can be modified in such a way that this leads to a modification of the expression of the endogenous desaturase). To generate a point mutation via homologous recombination, DNA-RNA hybrids, which are also known as chimeraplasty, and which are known from Cole-Strauss et al., 1999, Nucleic Acids Research 27(5):1323-1330 and Kmiec, Gene therapy, 1999, American Scientist, 87(3):240-247 can also be used.

In the vector for homologous recombination, the modified segment of the desaturase gene is flanked at its 5′ and 3′ end by additional nucleic acid of the desaturase gene, so that homologous recombination is possible between the exogenous desaturase gene which is present on the vector and an endogenous desaturase gene in a microorganism or plant. The additional flanking desaturase nucleic acid is sufficiently long for successful homologous recombination with the endogenous gene. Usually, several hundred base pairs up to kilobases of flanking DNA (both on the 5′ and on the 3′ end) are present in the vector (for a description of vectors for homologous recombination, see, for example, Thomas, K. R., and Capecchi, M. R. (1987) Cell 51:503 or for the recombination in Physcomitrella patens on cDNA basis, see Strepp et al., 1998, Proc. Natl. Acad. Sci. USA 95 (8):4368-4373). The vector is introduced into a microorganism or plant cell (for example by means of polyethylene glycol-mediated DNA), and cells in which the desaturase gene introduced has undergone homologous recombination with the endogenous desaturase gene are selected using techniques known in the art.

In another embodiment, recombinant organisms such as microorganisms can be generated which contain selected systems which allow regulated expression of the gene introduced. The inclusion of a desaturase gene in a vector, where it is placed under the control of the lac operon, allows, for example, expression of the desaturase gene only in the presence of IPTG. These regulatory systems are known in the art.

A host cell according to the invention, such as a prokaryotic or eukaryotic host cell, growing either in culture or in a field, can be used for producing (i.e. expressing) a desaturase. In plants, an alternative method can additionally be used by directly transferring DNA into developing flowers via electroporation or Agrobacterium-mediated gene transfer. Accordingly, the invention furthermore provides methods of producing desaturases using the host cells according to the invention. In one embodiment, the method encompasses growing the host cell according to the invention (into which a recombinant expression vector encoding a desaturase has been introduced or into whose genome a gene encoding a wild-type or modified desaturase has been introduced) in a suitable medium until the desaturase has been produced. In a further embodiment, the method encompasses isolating the desaturases from the medium or the host cell.

Host cells which are suitable in principle for taking up the nucleic acid according to the invention, the gene product according to the invention or the vector according to the invention are all prokaryotic or eukaryotic organisms. The host organisms which are used advantageously are organisms such as bacteria, fungi, yeasts, animal cells or plant cells. Further advantageous organisms are animals or, preferably, plants or parts thereof. Fungi, yeasts or plants are preferably used, especially preferably fungi or plants, very especially preferably plants such as oil crop plants which contain large amounts of lipid compounds, such as oilseed rape, evening primrose, canola, peanut, linseed, soybean, safflower, sunflower, borage, or plants such as maize, wheat, rye, oats, triticale, rice, barley, cotton, cassaya, pepper, tagetes, Solanaceae plants such as potato, tobacco, egg-plant and tomato, Vicia species, pea, alfalfa, bush plants (coffee, cacao, tea), Salix species, trees (oil palm, coconut) and perennial grasses and fodder crops. Especially preferred plants according to the invention are oil crop plants such as soybean, peanut, oilseed rape, canola, linseed, evening primrose, sunflower, safflower, trees (oil palm, coconut).

D. Isolated Desaturase

A further aspect of the invention relates to isolated desaturases and biologically active parts thereof. An “isolated” or “purified” protein or a biologically active part thereof is essentially free of cellular material when it is produced by recombinant DNA techniques, or free from chemical precursors or other chemicals when it is synthesized chemically. The term “essentially free of cellular material” encompasses desaturase preparations in which the protein is separated from cellular components of the cells in which it is produced naturally or recombinantly. In one embodiment, the term “essentially free of cellular material” encompasses desaturase preparations with less than approximately 30% (based on the dry weight) of non-desaturase (also referred to herein as “contaminating protein”), more preferably less than approximately 20% of non-desaturase, even more preferably less than approximately 10% of non-desaturase and most preferably less than approximately 5% of non-desaturase. If the desaturase or a biologically active part thereof has been produced recombinantly, it is also essentially free of culture medium, i.e. the culture medium amounts to less than approximately 20%, more preferably less than approximately 10% and most preferably less than approximately 5% of the volume of the protein preparation. The term “essentially free from chemical precursors or other chemicals” encompasses desaturase preparations in which the protein is separate from chemical precursors or other chemicals which are involved in the synthesis of the protein. In one embodiment, the term “essentially free of chemical precursors or other chemicals” encompasses desaturase preparations with less than approximately 30% (based on the dry weight) of chemical precursors or on-desaturase chemicals, more preferably less than approximately 20% of chemical precursors or non-desaturase chemicals, even more preferably less than approximately 10% of chemical precursors or non-desaturase chemicals and most preferably less than approximately 5% of chemical precursors or non-desaturase chemicals. In preferred embodiments, isolated proteins or biologically active parts thereof exhibit no contaminating proteins from the same organisms from which the desaturase originates. These proteins are usually produced by recombinant expression, for example, Phaeodactylum tricornutum desaturase in plants such as Physcomitrella patens or above-mentioned microorganisms, for example bacteria such as E. coli, Bacillus subtilis, C. glutamicum, fungi such as Mortierella, yeasts such as Saccharomyces, or ciliates such as Colpidium or algae such as Phaeodactylum.

An isolated desaturase according to the invention or a part thereof can also participate in the metabolism of compounds required for the synthesis of cell membranes in Phaeodactylum tricornutum or in the transport of molecules via these membranes. In preferred embodiments, the protein or the part thereof encompasses an amino acid sequence which has sufficient homology with an amino acid sequence of SEQ ID NO: 2, 4, 6 or 12 for the protein or part thereof to retain the ability to participate in the metabolism of compounds required for the synthesis of cell membranes in Phaeodactylum tricornutum or in the transport of molecules via these membranes. The part of the protein is preferably a biologically active part as described herein. In a further preferred embodiment, a desaturase according to the invention has one of the amino acid sequences shown in SEQ ID NO: 2, 4, 6 or 12. In a further preferred embodiment, the desaturase has an amino acid sequence which is encoded by a nucleotide sequence which hybridizes with a nucleotide sequence of SEQ ID NO: 1, 3, 5 or 11, for example under stringent conditions. In yet another preferred embodiment, the desaturase has an amino-acid sequence which is encoded by a nucleotide sequence which has at least approximately 50 to 60%, preferably at least approximately 60 to 70%, more preferably at least approximately 70 to 80%, 80 to 90%, 90 to 95%, and even more preferably at least approximately 96%, 97%, 98%, 99% or more homology with one of the amino acid sequences of SEQ ID NO: 2, 4, 6 or 18. The desaturase preferred according to the invention preferably also has at least one of the desaturase activities described herein. For example, a desaturase preferred according to the invention encompasses an amino acid sequence encoded by a nucleotide sequence which hybridizes with a nucleotide sequence of SEQ ID NO: 1, 3, 5 or 11, for example under stringent conditions, and which can participate in the metabolism of compounds required for the synthesis of cell membranes in Phaeodactylum tricornutum or in the transport of molecules via these membranes and is capable of introducing a double bond into a fatty acid with one, two, three or four double bonds and a chain length of C₁₈, C₂₀ or C₂₂.

In other embodiments, the desaturase is essentially homologous with an amino acid sequence of SEQ ID NO: 2, 4 or 6 and retains the functional activity of the protein of one of the sequences of SEQ ID NO: 2, 4 or 6, the amino acid sequence differing, however, owing to natural variation or mutagenesis as described in detail in the above subsection I. In a further embodiment, the desaturase is, accordingly, a protein encompassing an amino acid sequence which has at least approximately 50 to 60% homology, preferably approximately 60 to 70% homology and more preferably at least approximately 70 to 80%, 80 to 90%, 90 to 95% homology and most preferably at least approximately 96%, 97%, 98%, 99% or more homology with a complete amino acid sequence of SEQ ID NO: 2, 4 or 6 and has at least one of the desaturase activities described herein. In another embodiment, the invention relates to a complete Phaeodactylum tricornutum protein which is essentially homologous with a complete amino acid sequence of SEQ ID NO: 2, 4 or 6.

Biologically active parts of a desaturase encompass peptides encompassing amino acid sequences derived from the amino acid sequence of a desaturase, for example an amino acid sequence shown in SEQ ID NO: 2, 4 or 6 or the amino acid sequence of a protein which is homologous with a desaturase, which peptides have fewer amino acids than the full-length desaturase or the full-length protein which is homologous with a desaturase and have at least one activity of a desaturase. Biologically active parts (peptides, for example peptides with a length of, for example, 5, 10, 15, 20, 30, 35, 36, 37, 38, 39, 40, 50, 100 or more amino acids) usually encompass a domain or a motif with at least one activity of a desaturase. Moreover, other biologically active parts in which other regions of the protein are deleted can be generated by recombinant techniques and examined for one or more of the activities described herein. The biologically active parts of the desaturase preferably encompass one or more selected domains/motifs or parts thereof with biological activity.

Desaturases are preferably produced by recombinant DNA techniques. For example, a nucleic acid molecule encoding the protein is cloned into an expression vector (as described above), the expression vector is introduced into a host cell (as described above), and the desaturase is expressed in the host cell. The desaturase can then be isolated from the cells by a suitable purification scheme using standard techniques of protein purification. As an alternative to the recombinant expression, a desaturase, a desaturase polypeptide or a desaturase peptide can be synthesized chemically by means of standard techniques of peptide synthesis. Moreover, native desaturase can be isolated from cells (for example endotheliol cells), for example using an anti-desaturase antibody which can be raised by standard techniques, using a desaturase according to the invention or a fragment thereof.

The invention also provides chimeric desaturase proteins or desaturase fusion proteins. As used in the present context, a “chimeric desaturase protein” or “desaturase fusion protein” encompasses a desaturase polypeptide which is bound functionally to a non-desaturase polypeptide. A “desaturase polypeptide” refers to a polypeptide with an amino acid sequence which corresponds to a desaturase, whereas a “non-desaturase polypeptide” refers to a polypeptide with an amino acid sequence which corresponds to a protein which is essentially not homologous with the desaturase, for example a protein which differs from the desaturase and which originates from the same or another organism. Within the fusion protein, the term “linked functionally” is understood as meaning that the desaturase polypeptide and the non-desaturase polypeptide are fused to each other in such a way that both sequences fulfil the predicted function which has been ascribed to the sequence used. The non-desaturase polypeptide can be fused to the N terminus or the C terminus of the desaturase polypeptide. In one embodiment, the fusion protein is, for example, an EST-desaturase fusion protein in which the desaturase sequences are fused to the C terminus of the GST sequences. These fusion proteins can facilitate the purification of the recombinant desaturases. In a further embodiment, the fusion protein is a desaturase which has a heterologous signal sequence (N terminus). In specific host cells (for example mammalian host cells), the expression and/or secretion of a desaturase can be increased by using a heterologous signal sequence.

A chimeric desaturase protein or desaturase fusion protein according to the invention is produced by standard recombinant DNA techniques. For example, DNA fragments which encode different polypeptide sequences are ligated to each other in-frame using conventional techniques, for example by employing blunt ends or overhanging ends for ligation, restriction enzyme cleavage for providing suitable ends, filling up cohesive ends, as required, treatment with alkaline phosphatase to avoid undesired linkages, and enzymation ligation. In a further embodiment, the fusion gene can be synthesized by conventional techniques including DNA synthesizers. As an alternative, PCR amplification of gene fragments can be carried out using anchor primers which generate complementary overhangs between successive gene fragments which can subsequently be hybridized with each other and reamplified to give rise to a chimeric gene sequence (see, for example, Current Protocols in Molecular Biology, Ed. Ausubel et al., John Wiley & Sons: 1992). Moreover, a large number of expression vectors which already encode a fusion unit (for example a GST polypeptide) are commercially available. A desaturase-encoding nucleic acid can be cloned into such an expression vector so that the fusion unit is linked in-frame to the desaturase protein.

Desaturase homologs can be generated by mutagenesis, for example by specific point mutation or by truncating the desaturase. The term “homologs” as used in the present context refers to a variant form of the desaturase which acts as agonist or antagonist with the desaturase activity. A desaturase agonist can essentially retain the same activity as the desaturase, or some of the biological activities of the desaturase. A desaturase antagonist can inhibit one or more activities of the naturally occurring desaturase form, for example by competitive binding to an upstream or downstream element of the metabolic cascade for cell membrane components which encompass the desaturase, or by binding to a desaturase which mediates the transport of compounds via cell membranes, thus inhibiting translocation. In an alternative embodiment, desaturase homologs can be identified by screening combinatory libraries of desaturase mutants, for example truncated mutants, with regard to desaturase agonist or antagonist activity. In one embodiment, a variegated library of desaturase variants is generated at nucleic acid level by combinatory mutagenesis and encoded by a variegated genetic library. A variegated library of desaturase variants can be generated for example by enzymatic ligation of a mixture of synthetic oligonucleotides into gene sequences so that a degenerate set of potential desaturase sequences can be expressed as individual polypeptides or, alternatively, as a set of larger fusion proteins (for example for phage display) which comprise this set of desaturase sequences. There is a multiplicity of methods which can be used for generating libraries of potential desaturase homologs from a degenerate oligonucleotide sequence. The chemical synthesis of a degenerate gene sequence can be carried out in a DNA synthesizer, and the synthetic gene can then be ligated into a suitable expression vector. The use of the degenerate set of genes allows all sequences which encode the desired set of potential desaturase sequences to be provided in a mixture. Methods for the synthesis of degenerate oligonucleotides are known in the art (see, for example, Narang, S. A. (1983) Tetrahedron 39:3; Itakura et al. (1984) Annu. Rev. Biochem. 53:323; Itakura et al., (1984) Science 198:1056; Ike et al. (1983) Nucleic Acids Res. 11:477).

In addition, libraries of desaturase fragments can be used for generating a variegated population of desaturase fragments for screening and for the subsequent selection of homologs of a desaturase. In one embodiment, a library of fragments of the coding sequence can be generated by treating a double-strand PCR fragment of a coding desaturase sequence with a nuclease under conditions under which double-strand breaks only occur approximately once per molecule, denaturing the double-stranded DNA, renaturing the DNA with the formation of double-stranded DNA which can encompass sense/antisense pairs of various products with double-strand breaks, removal of single-stranded sections from newly formed duplices by treatment with S1 nuclease, and ligating the resulting fragment library into an expression vector. Using this method, an expression library can be derived which encodes N-terminal, C-terminal and internal desaturase fragments of various sizes.

A number of techniques for screening gene products in combinatory libraries which have been generated by point mutation or truncation and for screening cDNA libraries for gene products with a selected property are known in the art. These techniques can be adapted to rapid screening of the gene libraries which have been generated by combinatory mutagenesis of desaturase homologs. The most frequently used techniques for screening large gene libraries which can be subjected to high-throughput analysis usually encompass cloning the gene library into replicable expression vectors, transforming suitable cells with the resulting vector library, and expressing the combinatory genes under conditions under which detecting the desired activity facilitates the isolation of the vector encoding the gene whose product has been detected. Recursive ensemble mutagenesis (REM), a novel technique which increases the frequency of functional mutants in the libraries, can be used in combination with the screening assays for identifying desaturase homologs (Arkin and Yourvan (1992) Proc. Natl. Acad. Sci. USA 89:7811-7815; Delgrave et al. (1993) Protein Engineering 6(3):327-331).

A further known technique for modifying catalytic properties of enzymes or the genes encoding them is gene shuffling (see, for example, Stemmer, PNAS 1994, 91: 10747-10751, WO 97/20078 or WO 98/13487), which is a combination of gene fragments where this new combination can additionally be varied by erroneous polymerase chain reactions thus creating a high sequence diversity to be assayed. However, the prerequisite for using such an approach is a suitable screening system for testing the resulting gene diversity for functionality.

A screening method which identifies a PUFA-dependent enzyme activity or activities, is a prerequisite in particular for screening desaturase activities. As regards desaturase activities with a specificity for PUFAs, the toxicity of arachidonic acid in the presence of a toxic metabolyte (here: salicylic acid or salicylic acid derivatives) can be exploited in Mucor species which can be transformed with desired gene constructs by known transformation methods (Eroshin et al., Mikrobiologiya, Vol. 65, No.1 1996, pages 31-36), to carry out a growth-based primary screening. Resulting clones can then be analyzed for their lipid constituents by means of gas chromatography and mass spectroscopy in order to identify the nature and quantity of starting materials and products.

In a further embodiment, cell-based assays can be made use of for analyzing a variegated desaturase library using further processes known in the art.

E. Uses and Processes/Methods According to the Invention

The nucleic acid molecules, proteins, protein homologs, fusion proteins, primers, vectors and host cells described herein can be used in one or more of the processes/methods which follow: identification of Phaeodactylum and related organisms, genome mapping of organisms which are related to Phaeodactylum tricornutum, identification and localization of Phaeodactylum tricornutum sequences of interest, evolutionary studies, determination of desaturase protein regions required for the function, modulation of a desaturase activity, modulation of the metabolism of one or more cell membrane components, modulation of the transmembrane transport of one or more compounds, and modulation of the cellular production of a desired compound such as a fine chemical. The desaturase nucleic acid molecules according to the invention have a multiplicity of uses. Firstly, they can be used for identifying an organism as Phaeodactylum tricornutum or a close relative thereof. They can also be used for identifying the presence of Phaeodactylum tricornutum or of a relative thereof in a mixed population of microorganisms. The invention provides the nucleic acid sequences of a series of Phaeodactylum tricornutum genes; the presence or absence of this organism can be determined by screening the extracted genomic DNA of a culture of a uniform or mixed population of microorganisms under stringent conditions with a probe covering a region of a Phaeodactylum tricornutum gene or parts thereof, which gene is unique to this organism. Phaeodactylum tricornutum itself is used for the commercial production of polyunsaturated acids and is additionally suitable for the production of PUFAs, also in other organisms, in particular when it is intended for the resulting PUFAs also to be incorporated into the triacylglycerol fraction.

Furthermore, the nucleic acid and protein molecules according to the invention can act as markers for specific regions of the genome. This is suitable not only for mapping the genome, but also for functional Phaeodactylum tricornutum proteins. To identify the genome region to which a certain DNA-binding protein of Phaeodactylum tricornutum binds, it might be possible, for example, to fragment the Phaeodactylum tricornutum genome, and the fragments could be incubated with the DNA-binding protein. Those which bind the protein can additionally be screened with the nucleic acid molecules according to the invention, preferably with readily detectable markers; the binding of such a nucleic acid molecule to the genome fragment makes possible the localization of the fragment on the genome map of Phaeodactylum tricornutum and, if this is carried out repeatedly with different enzymes, facilitates a rapid determination of the nucleic acid sequence to which the protein binds. Moreover, the nucleic acid molecules according to the invention can have sufficient homology with the sequences of related species for these nucleic acid molecules to be able to act as markers for the construction of a genomic map in related fungi or algae.

The desaturase nucleic acid molecules according to the invention are also suitable for evolutionary studies and studies of the protein structure. The metabolic and transport processes in which the molecules according to the invention are involved are utilized by many prokaryotic and eukaryotic cells; the evolutionary degree of relatedness of the organisms can be determined by comparing the sequences of the nucleic acid molecules according to the invention with those which encode similar enzymes from other organisms. Accordingly, such a comparison allows the determination of which sequence regions are conserved and which are not conserved, and this may be helpful when determining regions of the protein which are essential for enzyme function. This type of determination is valuable for protein engineering studies and may provide a clue of how much mutagenesis the protein can tolerate without losing its function.

Manipulation of the desaturase nucleic acid molecules according to the invention can lead to the production of desaturases with functional differences to the wild-type desaturases. The efficiency or activity of these proteins can be improved, they can be present in the cell in larger numbers than usual, or their efficiency or activity can be reduced. An improved efficiency or activity means, for example, that the enzyme has a higher selectivity and/or activity, preferably an activity which is at least 10% higher, especially preferably an activity which is at least 20% higher, very especially preferably an activity which is at least 30% higher than that of the original enzyme.

There exists a series of mechanisms by which modification of a desaturase according to the invention can directly affect the yield, production and/or production efficiency of a fine chemical comprising such a modified protein. Obtaining fine chemical compounds from cultures of ciliates, algae or fungi on a large scale is significantly improved when the cell secretes the desired compounds, since these compounds can be isolated readily from the culture medium (in contrast to extraction from the biomass of the cultured cells). Otherwise, purification can be improved when the cell stores compounds in-vivo in a specialized compartment with a sort of concentration mechanism. In plants which express desaturases, an increased transport may lead to better distribution within the plant tissue and the plant organs. Increasing the number or the activity of transporter molecules which export fine chemicals from the cell may allow the quantity of the fine chemicals produced, which is present in the extracellular medium, to be increased, thus facilitating harvesting and purification or, in the case of plants, more efficient distribution. In contrast, increased amounts of cofactors, precursor molecules and intermediates for the suitable biosynthetic pathways are required for efficient overproduction of one or more fine chemicals. Increasing the number and/or the activity of transporter proteins involved in the import of nutrients such as carbon sources (i.e. sugars), nitrogen sources (i.e. amino acids, ammonium salts), phosphate and sulfur can improve the production of a fine chemical owing to the elimination of all limitations of the nutrients available in the biosynthetic process. Fatty acids such as PUFAs and lipids comprising PUFAs are desirable fine chemicals themselves; optimizing the activity or increasing the number of one or more desaturases according to the invention involved in the biosynthesis of these compounds, or disrupting the activity of one or more desaturases involved in the catabolism of these compounds, can thus increase the yield, production and/or production efficiency of fatty acids and lipid molecules in ciliates, algae, plants, fungi, yeasts or other microorganisms.

The manipulation of one or more desaturase genes according to the invention can likewise lead to desaturases with modified activities which indirectly affect the production of one or more desired fine chemicals from algae, plants, ciliates or fungi. The normal biochemical metabolic processes leaked, for example, to the production of a multiplicity of waste products (for example hydrogen peroxide and other reactive oxygen species) which can actively disrupt these metabolic processes (for example, peroxynitrite is known to nitrate tyrosine side chains, thus inactivating some enzymes with tyrosin in the active center (Groves, J. T. (1999) Curr. Opin. Chem. Biol. 3(2);226-235)). While these waste products are normally excreted, the cells used for fermentative production on a large scale are optimized for the overproduction of one or more fine chemicals and can therefore produce more waste products than is customary for a wild-type cell. Optimizing the activity of one or more desaturases according to the invention involved in the export of waste molecules allows the improvement of the viability of the cell and the maintenance of an efficient metabolic activity. Also, the presence of high intracellular amounts of the desired fine chemical can in fact be toxic to the cell, so that the viability of the cell can be improved by increasing the ability of the cell to secrete these compounds.

Furthermore, the desaturases according to the invention can be manipulated in such a way that the relative amounts of various lipids and fatty acid molecules are modified. This can have a decisive effect on the lipid composition of the cell membrane. Since each lipid type has different physical properties, a modification of the lipid composition of the membrane can significantly modify membrane fluidity. Changes in membrane fluidity can affect the transport of molecules via the membrane which, as explained above, can modify the export of waste products or of the fine chemical produced or the import of nutrients which are required. These changes in membrane fluidity can also have a decisive effect on cell integrity; cells with comparatively weaker membranes are more susceptible to abiotic and biotic stress conditions which can damage or kill the cell. Manipulation of desaturases involved in the production of fatty acids and lipids for membrane synthesis so that the resulting membrane has a membrane composition which is more susceptible to the environmental conditions prevailing in the cultures used for the production of fine chemicals should allow more cells to survive and multiply. Larger numbers of producing cells should manifest themselves in greater yields, higher production or higher production efficiency of the fine chemical from the culture.

The abovementioned mutagenesis strategies for desaturases intended to lead to elevated yields of a fine chemical are not to be construed as limiting; variations of these strategies are readily obvious to the skilled worker. Using these mechanisms, and with the aid of the mechanisms disclosed herein, the nucleic acid and protein molecules according to the invention can be used for generating algae, ciliates, plants, animals, fungi or other microorganisms such as C. glutamicum, which express mutated desaturase nucleic acid and protein molecules so that the yield, production and/or production efficiency of a desired compound is improved. This desired compound can be any natural product of algae, ciliates, plants, animals, fungi or bacteria which encompasses the end products of biosynthetic pathways and intermediates of naturally occurring metabolic pathways, and also molecules which do not naturally occur in the metabolism of these cells, but which are produced by the cells according to the invention.

A further embodiment according to the invention is a process for the production of PUFAs, which comprises culturing an organism which contains a nucleic acid according to the invention, a gene construct according to the invention or a vector according to the invention which encode a polypeptide which elongates C₁₈-, C₂₀- or C₂₂-fatty acids with at least two double bonds in the fatty acid molecule by at least two carbon atoms under conditions under which PUFAs are produced in the organism. PUFAs produced by this process can be isolated by harvesting the organisms either from the culture in which they grow or from the field, and disrupting and/or extracting the harvested material with an organic solvent. The oil, which contains lipids, phospholipids, sphingolipids, glycolipids, triacylglycerols and/or free fatty acids with a higher PUFA content can be isolated from this solvent. The free fatty acids with a higher PUFA content can be isolated by basic or acid hydrolysis of the lipids, phospholipids, sphingolipids, glycolipids and triacylglycerols. A higher PUFA content means at least 5%, preferably 10%, especially preferably 20%, very especially preferably 40% more PUFAs than the original organism which does not have additional nucleic acid encoding the desaturase according to the invention.

The PUFAs produced by this process are preferably C₁₈- or C₂₀₋₂₂-fatty acid molecules with at least two double bonds in the fatty acid molecule, preferably three, four, in combination with a further elongase and a Δ4-desaturase five or six double bonds. These C₁₈- or C₂₀₋₂₂-fatty acid molecules can be isolated from the organism in the form of an oil, lipid or a free fatty acid. Examples of suitable organisms are those mentioned above. Preferred organisms are transgenic plants.

An embodiment according to the invention are oils, lipids or fatty acids or fractions thereof which have been prepared by the above-described process, especially preferably an oil, a lipid or a fatty acid composition comprising PUFAs and originating from transgenic plants.

A further embodiment according to the invention is the use of the oil, lipid or fatty acid composition in feeds, foods, cosmetics or pharmaceuticals.

The invention further relates to a method of identifying an antagonist or agonist of desaturases, comprising

a) contacting the cells which express the polypeptide of the present invention with a candidate substance;

b) testing the desaturate activity;

c) comparing the desaturase activity with a standard activity in the absence of the candidate material, where an increase in the desaturase activity beyond the standard indicates that the candidate material is an agonist and a reduction in the desaturase activity indicates that the candidate material is an antagonist.

The candidate substance mentioned can be a substance which has been synthesized chemically or produced by microbes and can occur, for example, in cell extracts of, for example, plants, animals or microorganisms. Moreover, the substance mentioned, while being known in the prior art, may not be known as yet as increasing or reversing the activity of the desaturases. The reaction mixture can be a cell-free extract or encompass a cell or cell culture. Suitable methods are known to the skilled worker and are described in general terms for example in Alberts, Molecular Biology the cell, 3rd Edition (1994), for example Chapter 17. The substances mentioned can be added for example to the reaction mixture or the culture medium or else injected into the cells or sprayed onto a plant.

When a sample comprising an active substance by the method according to the invention has been identified, it is either possible directly to isolate the substance from the original sample or else the sample can be divided into various groups, for example when they consist of a multiplicity of various components, in order to reduce the number of the various substances per sample and then to repeat the method according to the invention with such a “subset” of the original sample. Depending on the complexity of the sample, the above-described steps can be repeated repeatedly, preferably until the sample identified in accordance with the method according to the invention only still contains a small number of substances, or only one substance. Preferably, the substance identified in accordance with the method according to the invention, or derivatives of the substance, are formulated further so that it is suitable for use in plant breeding or in plant cell or tissue culture.

The substances which have been assayed and identified in accordance with the method according to the invention can be: expression libraries, for example cDNA expression libraries, peptides, proteins, nucleic acids, antibodies, small organic substances, hormones, PNAs or the like (Milner, Nature Medicin 1 (1995), 879-880; Hupp, Cell. 83 (1995), 237-245; Gibbs, Cell. 79 (1994), 193-198 and references cited therein). These substances can also be functional derivatives or analogs of the known inhibors or activators. Methods of preparing chemical derivatives or analogs are known to the skilled worker. The derivatives and analogs mentioned can be assayed in accordance with prior-art methods. Moreover, computer-aided design or peptidomimetics can be used for producing suitable derivatives and analogs. The cell or the tissue which can be used for the method according to the invention is preferably a host cell according to the invention, a plant cell according to the invention or a plant tissue as described in the abovementioned embodiments.

Accordingly, the present invention also relates to a substance which has been identified in accordance with the above methods according to the invention. The substance is, for example, a homolog of the desaturases according to the invention. Homologs of the desaturases can be generated by mutagenesis, for example by point mutation or deletion of the desaturases. The term “homolog” as used in the present context denotes a variant form of the desaturases which acts as agonist or antagonist for the activity of the desaturases. An agonist can have essentially the same or part of the biological activity of the desaturases. An antagonist of the desaturases can inhibit one or more activities of the naturally occurring forms of the desaturases, for example can undergo competitive banding to a downstream or upstream member of the fatty acid synthesis metabolic pathways, which include the desaturases, or can bind to desaturases and thus reduce or inhibit the activity.

Moreover, the present invention also relates to an antibody or a fragment thereof as are described herein, which antibody or fragment inhibits the activity of the desaturases according to the invention.

In one aspect, the present invention relates to an antibody which specifically recognizes, or binds to, the above-described agonist or antagonist according to the invention.

A further aspect relates to a composition comprising the antibody, the stop identified by the method according to the invention or the antisense molecule.

In a further embodiment, the present invention relates to a kit comprising the nucleic acid according to the invention, the gene construct according to the invention, the amino acid sequence according to the invention, the antisense nucleic acid molecule according to the invention, the antibody and/or composition according to the invention, an antagonist or agonist prepared by the method according to the invention, and/or oils, lipids and/or fatty acids according to the invention or a fraction thereof. Equally, the kit can comprise the host cells, organisms, plants according to the invention or parts thereof, harvestable parts of the plants according to the invention or propagation material or else the antagonist or agonist according to the invention. The components of the kit of the present invention can be packaged in suitable containers, for example with or in buffers or other solutions. One or more of the abovementioned components may be packaged in one and the same container. In addition, or as an alternative, one or more of the abovementioned components can be adsorbed onto a solid surface, for example nitrocellulose filters, glass sheets, chips, nylon membranes or microtiter plates. The kit can be used for any of the methods and embodiments described herein, for example for the production of host cells, transgenic plants, for the detection of homologous sequences, for the identification of antagonists or agonists and the like. Furthermore, the kit can comprise instructions for the use of the kit for one of the abovementioned applications.

The present invention is illustrated in greater detail by the examples which follow, and which must not be construed as limiting. The content of any references, patent applications, patents and published patent applications cited in the present patent application is herewith incorporated by reference.

### EXAMPLES Section

### Example 1

### General methods

a) General Cloning Methods:

Cloning methods such as, for example, restriction cleavages, agarose gel electrophoresis, purification of DNA fragments, transfer of nucleic acids to nitrocellulose and nylon membranes, linkage of DNA fragments, transformation of Escherichia coli and yeast cells, the culture of bacteria and the sequence analysis of recombinant DNA were carried out as described in Sambrook et al. (1989) (Cold Spring Harbor Laboratory Press: ISBN 0-87969-309-6) or Kaiser, Michaelis and Mitchell (1994) “Methods in Yeast Genetics” (Cold Spring Harbor Laboratory Press: ISBN 0-87969-451-3). The transformation and culture of algae such as Chlorella or Phaeodactylum are carried out as described by El-Sheekh (1999), Biologia Plantarum 42:209-216; Apt et al. (1996) Molecular and General Genetics 252 (5):872-9.

b) Chemicals

Unless otherwise specified in the text, the chemicals used were obtained in analytical quality from Fluka (Neu-Ulm), Merck (Darmstadt), Roth (Karlsruhe), Serva (Heidelberg) and Sigma (Deisenhofen). Solutions were supplied using pure pyrogen-free water, referred to in the following text as H₂O, on a Milli-Q water system water purification unit (Millipore, Eschborn). Restriction endonucleases, DNA-modifying enzymes and molecular biology kits were obtained from AGS (Heidelberg), Amersham (Braunschweig), Biometra (Gottingen), Boehringer (Mannheim), Genomed (Bad Oeynhausen), New England Biolabs (Schwalbach/Taunus), Novagen (Madison, Wis., USA), Perkin-Elmer (Weiterstadt), Pharmacia (Freiburg), Qiagen (Hilden) and Stratagene (Amsterdam, the Netherlands). Unless otherwise specified, they were used following the manufacturer's instructions.

c) Cell Material

The isolated nucleic acid sequences according to the invention are present in the genome of a Phaeodactylum tricornutum UTEX646 strain, which is available from the algae collection of the University of Texas, Austin.

Phaeodactylum tricornutum was cultured at 25° C. at a light/dark photo period of 14:10 hours at 22° C. and 35 microEinstein (corresponds to micromol of photons per square meter and second) in glass tubes into which air was passed from the bottom.

The culture medium used for Phaeodactylum tricornutum was the f/2 culture medium supplemented with 10% organic medium of Guillard, R. R. L. (1975; Culture of phytoplankton for feeding marine invertebrates. In: Smith, W. L. and Chanley, M. H. (Eds.) Culture of marine Invertebrate animals, NY Plenum Press, pp. 29-60.):

It comprises

995.5 ml of (artificial) sea water

1 ml of NaNO₃ (75 g/l), 1 ml of NaH₂PO₄ (5 g/l), 1 ml of trace element solution, 1 ml of Tris/Cl pH 8.0, 0.5 ml of f/2 vitamin solution

Trace element solution: Na₂EDTA (4.36 g/l), FeCl₃ (3.15 g/l), Primary trace elements: CuSO₄ (10 g/l), ZnSO₄ (22 g/l), CoCl₂ (10 g/l), MnCl₂ (18 g/l), NaMoO4 (6.3 g/l) f/2 vitamin solution: biotin: 10 mg/l, thiamine 200 mg/l, vitamin B120.1 mg/l

org medium: sodium acetate (1 g/l), glucose (6 g/l), sodium succinate (3 g/l), Bacto-tryptone (4 g/l), yeast extract (2 g/l)

### Example 2

### Isolation of Total DNA from Phaeodactylum tricornutum UTEX646 for Hybridization Experiments

The details of the isolation of total DNA refer to the work-up of plant material with a fresh weight of one gram.

CTAB buffer: 2% (w/v) N-acetyl-N,N,N-trimethylammonium bromide (CTAB); 100 mM Tris-HCl, pH 8.0; 1.4 M NaCl; 20 mM EDTA.

N-Laurylsarcosine buffer: 10% (w/v) of N-laurylsarcosine; 100 mM Tris-HCl, pH 8.0; 20 mM EDTA.

Phaeodactylum tricornutum cell material was triturated in a mortar under liquid nitrogen to give a fine powder which was transferred into 2 ml Eppendorf vessels. The frozen plant material was then covered with a layer of 1 ml of break buffer (1 ml of CTAB buffer, 100 ml of N-laurylsarcosine buffer, 20 ml of 1-mercaptoethanol and 10 ml of proteinase K solution, 10 mg/ml) and incubated at 60° C. for one hour with continuous shaking. The homogenate obtained was distributed into two Eppendorf vessels (2 ml) and extracted twice by shaking with an equal volume of chloroform/isoamyl alcohol (24:1). For phase separation, centrifugation was carried out at 8 000×g and RT (=room temperature =−23° C.) for 15 minutes in each case. The DNA was then precipitated for 30 minutes at −70° C. using ice-cold isopropanol. The precipitated DNA was sedimented for 30 minutes at 10 000 g at 4° C. and resuspended in 180 ml of TE buffer (Sambrook et al., 1989, Cold Spring Harbor Laboratory Press: ISBN 0-87969-309-6). For further purification, the DNA was treated with NaCl (final concentration 1.2 M) and reprecipitated for 30 minutes at −70° C. using twice the volume of absolute ethanol. After a wash step with 70% strength ethanol, the DNA was dried and subsequently taken up in 50 ml of H₂O+RNase (final concentration 50 mg/ml). The DNA was dissolved overnight at 4° C., and the RNase cleavage was subsequently carried out for 1 hour at 37° C. The DNA was stored at 4° C.

### Example 3

### Isolation of Total RNA and poly(A)⁺ RNA from plants and Phaeodactylum tricornutum

Total RNA was isolated from plants such as linseed and oilseed rape and the like by a method described by Logemann et al. (1987, Anal. Biochem. 163, 21). The total RNA from moss can be obtained from protonema tissue using the GTC method (Reski et al., 1994, Mol. Gen. Genet., 244:352-359).

RNA Isolation of Phaeodactylum tricornutum:

Frozen samples of algae (−70° C.) were triturated in an ice-cold mortar under liquid nitrogen to give a fine powder. 2 volumes of homogenization medium (12.024 g of sorbitol, 40.0 ml of 1M Tris-HCl, pH 9 (0.2 M); 12.0 ml of 5 M NaCl (0.3 M), 8.0 ml of 250 mM EDTA, 761.0 mg of EGTA, 40.0 ml of 10% SDS were made up to 200 ml with H₂O and the pH was brought to 8.5) and 4 volumes of phenol with 0.2% mercaptoethanol were added to the frozen cell powder at 40 to 50° C. while mixing thoroughly. Then, 2 volumes of chloroform were added and the mixture was stirred vigorously for 15 minutes. The mixture was centrifuged for 10 minutes at 10 000 g and the aqueous phase was extracted with phenol/chloroform (2 volumes) and subsequently extracted with chloroform.

The resulting volume of the aqueous phase was treated with 1/20 volume of 4 M sodium acetate (pH 6) and 1 volume of isopropanol (ice-cold), and the nucleic acids were precipitated overnight at −20° C. The mixture was then centrifuged for 30 minutes at 10 000 g and the supernatant pipetted off. This was followed by a wash step with 70% strength EtOH and another centrifugation. The sediment was taken up in Tris-borate buffer (80 mM Tris-borate buffer, 10 mM EDTA, pH 7.0). The supernatant was then treated with ⅓ volume of 8 M LiCl, mixed and incubated for 30 minutes at 4° C. After recentrifugation, the sediment was washed with 70% strength ethanol, centrifuged and the sediment was dissolved in RNase-free water.

Poly(A)⁺ RNA was isolated using Dyna Beads® (Dynal, Oslo, Norway) following the instructions in the manufacturer's protocol.

After the RNA or poly(A)⁺ RNA concentration had been determined, the RNA was precipitated by adding 1/10 volume of 3 M sodium acetate, pH 4.6, and 2 volumes of ethanol and stored at −70° C.

For the analysis, 20 μg portions of RNA were separated in a formaldehyde-containing 1.5% strength agarose gel and transferred to nylon membranes (Hybond, Amersham). Specific transcripts were detected as described by Amasino ((1986) Anal. Biochem. 152, 304)).

### Example 4

### Construction of the cDNA Library

To construct the cDNA library from Phaeodactylum tricornutum, the first-strand synthesis was carried out using murine leukaemia virus reverse transcriptase (Roche, Mannheim, Germany) and oligo-d(T) primers, while the second-strand synthesis was carried out by incubation with DNA polymerase I, Klenow enzyme and RNase H cleavage at 12° C. (2 hours), 16° C. (1 hour) and 22° C. (1 hour). The reaction was quenched by incubation at 650C (10 minutes) and subsequently transferred to ice. Double-stranded DNA molecules were made blunt-ended with T4 DNA polymerase (Roche, Mannheim) at 37° C. (30 minutes). The nucleotides were removed by extraction with phenol/chloroform and Sephadex G50 spin columns. EcoRI/XhoI adapters (Pharmacia, Freiburg, Germany) were ligated to the cDNA ends by means of T4 DNA ligase (Roche, 12° C., overnight), recut with XhoI and phosphorylated by incubation with polynucleotide kinase (Roche, 37° C., 30 min). This mixture was subjected to separation on a low-melting agarose gel. DNA molecules with over 300 base pairs were eluted from the gel, extracted with phenol, concentrated on Elutip D columns (Schleicher and Schull, Dassel, Germany) and ligated to vector arms and packaged into lambda-ZAP-Express phages using the Gigapack Gold kit (Stratagene, Amsterdam, the Netherlands), using the manufacturer's material and following their instructions.

### Example 5

### DNA Sequencing and Computer Analysis

cDNA libraries as described in Example 4 were used for DNA sequencing by standard methods, in particular the chain termination method using the ABI PRISM Big Dye Terminator Cycle Sequencing Ready Reaction Kit (Perkin-Elmer, Weiterstadt, Germany). Sequencing of random clones which had been singled out was carried out following preparative plasmid preparation from cDNA libraries via in-vivo mass excision and retransformation of DH10B on agar plates (details on materials and protocol: Stratagene, Amsterdam, the Netherlands). Plasmid DNA was prepared from E. coli cultures grown overnight in Luria broth supplemented with ampicillin (see Sambrook et al. (1989) (Cold Spring Harbor Laboratory Press: ISBN 0-87969-309-6)) using a Qiagen DNA preparation robot (Qiagen, Hilden) following the manufacturer's protocols. Sequencing primers with the following nucleotide sequences were used:

5′-CAGGAAACAGCTATGACC-3′
5′-CTAAAGGGAACAAAAGCTG-3′
5′-TGTAAAACGACGGCCAGT-3′

The sequences were processed and annotated using the EST-MAX standard software package, which is commercially available from Bio-Max (Munich, Germany). Exploiting comparative algorithms, and using the search sequence shown in SEQ ID NO: 8, homologous genes were searched for using the BLAST program (Altschul et al. (1997) “Gapped BLAST and PSI-BLAST: a new generation of protein database search programs”, Nucleic Acids Res. 25:3389-3402). Two sequences from Phaeodactylum tricornutum with homologies with the Physcomitrella patens search sequence were characterized in greater detail.

### Example 5a

### Isolation of Phaeodactylum tricornutum Desaturases via Polymerase Chain Reaction with the Aid of Degenerate Oligonucleotides

Published desaturases allow motifs to be identified which are typical of Δ5- and Δ6-desaturases. Oligonucleotide sequences with possible variations are shown in the following text. Underneath the oligonucleotide sequence, the amino acid from which the base combination can be derived is shown in the one-letter code. For example, A/G means that either an A or a G is randomly incorporated at this position in the oligonucleotide when the unit is synthesized, since the base triplet derived from the corresponding amino acid can either be AAA or AAG. The DNA sequence may also contain an inosine (i) if the determination of a base at this position permits three or four different bases owing to the genetic code. The following sequences and primers can be used:

| 5′-forward primer: | |
|---|---|
| F1a: | TGG TGG AA A/G TGG AAi CA T/C AA |
| F1b: | TGG TGG AA A/G TGG ACi CA T/C AA |
| F1a: | W W K W N/T H K/N |
| F1b: | W W K W K H K/N |
| F2a: | Gi TGG AA A/G GAi A/C Ai CA T/C AA |
| F2b: | Gi TGG AA A/G TTG A/C Ai CA T/C AA |
| F2a: | G/W W K E/D K/Q/N H K/N |
| F2b: | G/W W K W K/Q/N H K/N |
| F3a: | T A/T i TTG AAi A/C A A/G C/A G/A i CA |
| F3b: | T A/T i TTG AAi A/C A A/G CAi CA |
| F3a: | W W K/N H/N R/Q H |
| F3b: | Y W K/N H/N R/Q H |
| F4a: | GTi TGG A A/T G/A GA A/G CA A/G CA |
| F4b: | GTi TGG A A/T G/A A/T A T/C CA A/C CA |
| F4a: | V W K/M E Q H |
| F4b: | V W K/M N/Y Q H |
| F5a1: | CA T/C TA T/C TGG AA A/G AA T/C CA G C |
| F5a1: | CA T/C TA T/C TGG AA A/G AA T/C CA A C |
| F5a1: | H Y W K N Q H/Q |
| F6a: | TTG TTG AAi A/C A A/G AA i CA T/C AA |
| F6a: | W W K/N H/N K/N H K/N |
| 3′- reverse primer | |
| R1b: | GG A/G AA/iAG G/A TG G/A TG T/C TC |
| R1b: | GG A/G AA iAA G/A TG C/A TG T/C TC |
| R1a: | P F L H H E |
| R1b: | P F F H H E |
| R2a1: | AA iAG A/G TG A/G TG iA C/T iA/G T/C TG |
| R2a2: | AA T/C AA A/G TG A/G TG iA C/T iA/G T/C TG |
| R2a1: | F L H H V/I V/A Q |
| R3a1: | AT iTG iGG A/G AA iAA A/G TG A/G TG |
| R3a2: | AT A/G TT iGG A/G AA iAA A/G TG A/G TG |
| R3a3: | AT iTG iGG A/G AA iAG A/G TG A/G TG |
| R3a4: | AT A/G TT iGG A/G AA iAG A/G TG A/G TG |
| R3a1: | I/M H/Q P F F H H |
| R3a2: | I/M N P F L H H |
| R4a1: | CT iGG A/G AA iA A/G A/G TG A/G TG |
| R4a2: | GA iGG A/G AA iA A/G A/G TG A/G TG |
| R4a3: | GT iGG A/G AA iA A/G A/G TG A/G TG |
| R4a1: | = T/R/S P F F/L H H |
| R5a1: | AA iAA A/G TG A/G TG T/C TC T/A/G AT T/C TG |
| R5a2: | AA iAG A/G TG A/G TG T/C TC T/A/G AT T/C TG |
| R5a1: | F F H H E I Q |
| R5a2: | F L H H E I Q |
| R6a1: | T iGG iA A/G iAA A/G TG A/G TG iAC |
| R6a1: | T iGG iA A/G iAG A/G TG A/G TG iAC |
| R6a1: | T/N P L F/L H H V |

Owing to various possibilities of variations, a large number of derived oligonucleotides are possible, but surprisingly it has been found that oligonucleotides shown can be particularly suitable for isolating desaturases.

The primers can be employed for polymerase chain reactions in all combinations. Individual combinations allowed desaturase fragments to be isolated when the following conditions were taken into consideration: for PCR reactions, in each case 10 nMol of primer and 10 ng of a plasmid library obtained by in-vivo excision were employed. It was possible to isolate the plasmid library from the phage library following the protocols of the manufacturer (Stratagene). The PCR reaction was carried out in a thermocycler (Biometra) using Pfu DNA polymerase (Stratagene) and the following temperature program: 3 minutes at 96° C. followed by 35 cycles with 30 seconds at 96° C., 30 seconds at 55° C. and 1 minute at 72° C. After the first step at 55° C., the annealing temperature was lowered stepwise by in each case 3° C., and, after the fifth cycle, an annealing temperature of 40° C. was retained. Finally, a 10-minute-cycle at 72° C. was carried out, and the reaction was stopped by cooling to 4° C.

The primer combinations F6a and R4a2 are shown underlined in the text, and it was possible to exploit them successfully for isolating a desaturase fragment. It was possible to verify the resulting fragment by sequencing; it showed homologies with Streptomyces coelicolor desaturase with the Genbank Accession No. T36617. The homology was obtained with the aid of the BLASTP program. The alignment is shown in FIG. 4. Identities of 34% and a homology of 43% with sequence T36617 were revealed. The DNA fragment was employed in accordance with the invention as shown in Example 7 in a hybridization experiment for isolating a full-length gene under standard conditions.

The coding region of a DNA sequence isolated in this way was obtained by translating the genetic code into a polypeptide sequence. SEQ ID NO: 3 shows a sequence 1434 base pairs in length which was isolated by the method described. The sequence has a start codon in positions 1 to 3 and a stop codon in positions 1432-1434 and it was possible to translate it into a polypeptide 477 amino acids in length. In the alignment with the gene sequence described in WO 98/46763, it was found that a nonidentical, but homologous, Phaeodactylum tricornutum fragment encoding 87 amino acids had previously been described. However, WO 98/46763 discloses neither a complete, functionally active desaturase nor position or substrate specificity. This is also made clear by the fact that homologies with both the Δ5- and Δ6-desaturase from Mortierella alpina are reported without indicating a specific function. The sequence according to the invention, in contrast, encodes a functionally active Δ6-acyl lipid desaturase.

### Example 6

### Identification of DNA Sequences Encoding Phaeodactylum tricornutum Desaturases

The full-length sequence of the Δ6-acyl lipid desaturase Pp_des6 AJ222980 (NCBI Genbank Accession No.) from the moss Physcomitrella patens (see also Table 1) and the Δ12-acyl lipid desaturase sequence (Table 1, see Ma_desl2) from Mortierella alpina AF110509 (AF110509 NCBI Genbank Accession No.) were employed for sequence alignment with the aid of the TBLASTN search algorithm.

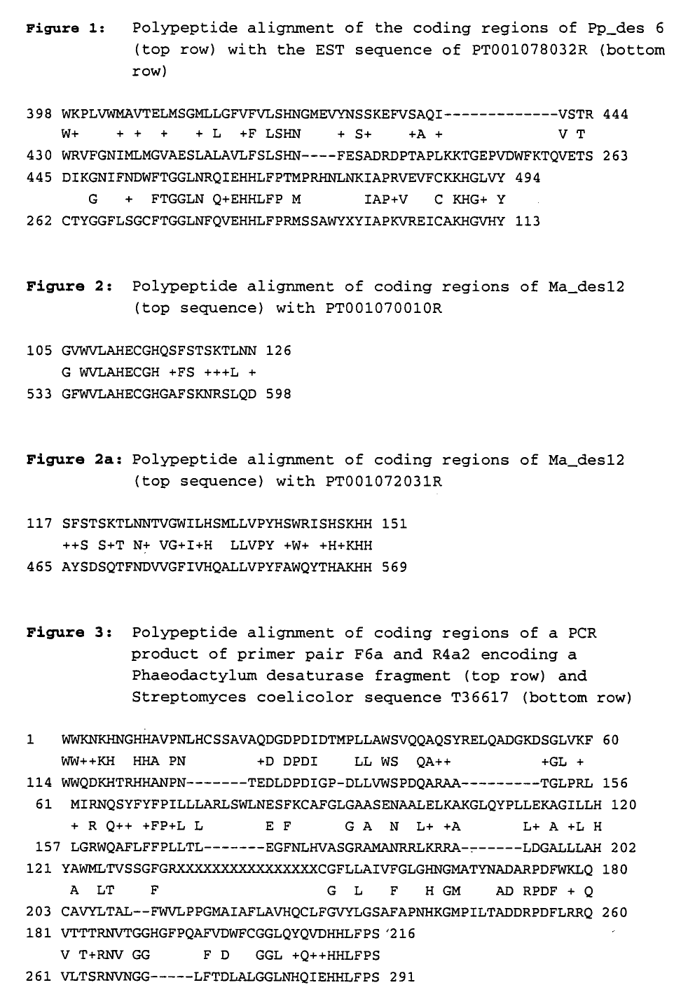

The EST sequences PT0010070010R, PT001072031R and PT001078032R were first considered as target gene among further candidate genes owing to weak homologies with the search sequences from Physcomitrella and Mortierella. FIGS. 1, 2 and 2a show the result of the two EST sequences found. The sequences found are part of the nucleic acids according to the invention of SEQ ID NO: 1 (gene name: Pt_des5, inventors' own database No. PT001078032R), SEQ ID NO: 5. (gene name: Pt_des12, inventors' own database No. PT0010070010R) and SEQ ID NO: 11 (gene name: Pt_desl2.2, inventor's own database No. PT001072031R). Letters indicate identical amino acids, while the plus symbol indicates a chemically similar amino acid. The identities and homologies of all sequences found in accordance with the invention can be seen from the summary in Table 2.

Desaturases can have cytochrome b5 domains which also occur in other genes which do not code for desaturases. Thus, cytochrome b5 domains show high homologies, even though the gene functions are different. Within weakly conserved regions, desaturases can only be identified as putative candidate genes and must be tested for the enzyme activity and position specificity of the enzymatic function. For example, various hydroxylases, acetylenases and epoxygenases, like desaturases, also show histidine box motifs, so that a specific function must be proven experimentally and only the additional verification of the double bond makes possible a guaranteed enzyme activity and position specificity of a desaturase. Surprisingly, it has been found that Δ6- and Δ5-desaturases according to the invention have particularly suitable substrate specificities and are particularly suitable for being exploited, in combination with a Physcomitrella Δ6-elongase, for producing polyunsaturated fatty acids such as arachidonic acid, eicosapentaenoic acid and docosahexanoic acid.

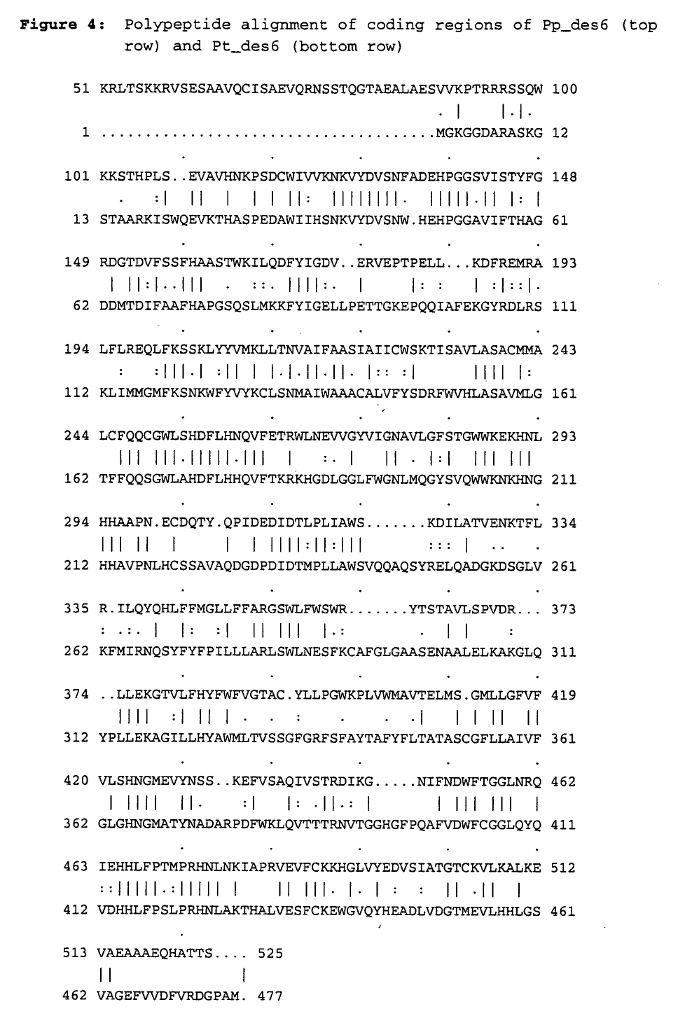

Sequencing of the full cDNA fragment from clone PT001078032R revealed a sequence 1652 base pairs in length. The sequence encodes a polypeptide of 469 amino acids shown in SEQ ID NO: 2. It was obtained by translating the genetic code of SEQ ID NO: 1 with a start codon in base pair position 115-117 and with a stop codon in base pair position 1522-1524. The clone comprises a complete desaturase polypeptide, as can be seen from the sequence alignment in FIG. 3. Lines denote identical amino acids, while colons and dots represent chemically exchangeable, i.e. chemically equivalent, amino acids. The alignment was carried out using Henikoff & Henikoff's BLOSUM62 substitution matrix ((1992) Amino acid substitution matrices from protein blocks. Proc. Natl. Acad. Sci. USA 89: 10915-10919). Parameters used: Gap Weight: 8; Average Match: 2.912, Length Weight: 2, Average Mismatch: -2.003.

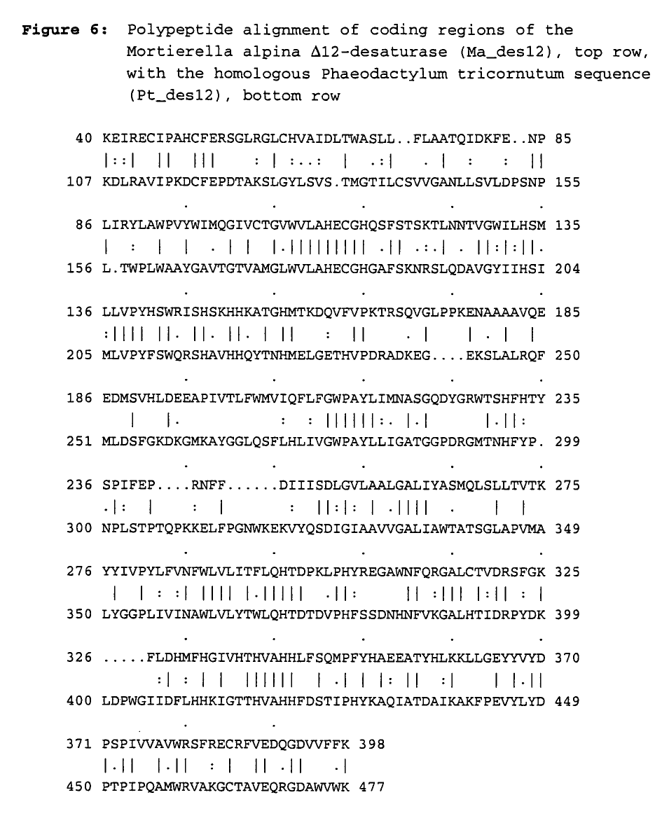

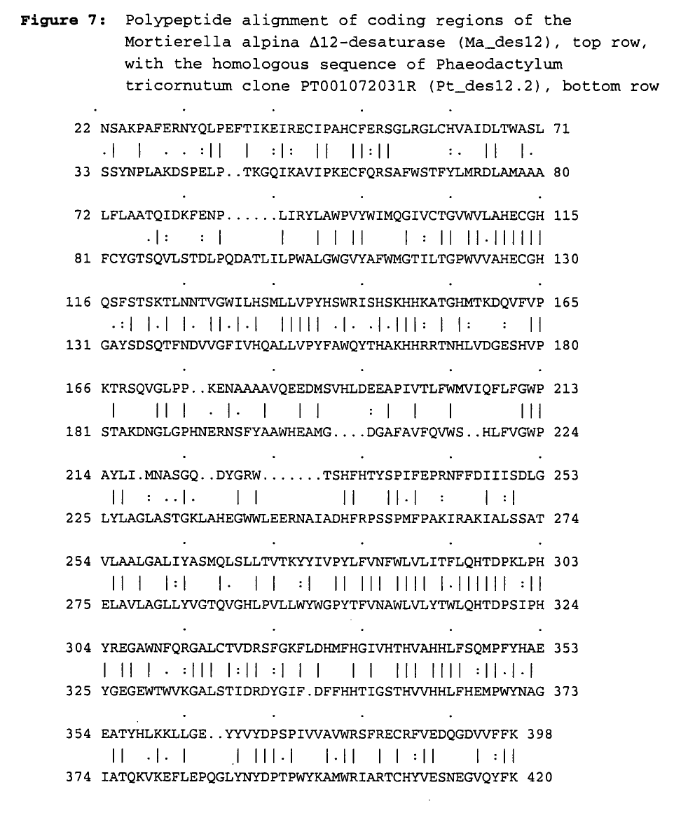

FIG. 6 and FIG. 7 show the alignment of the MA_des12 peptide sequence with the sequences found.

Sequencing of the complete cDNA fragment from clone PT0010070010R revealed a sequence 1651 base pairs in length and shown in SEQ ID NO: 5 with a start codon in position 67-69 and a stop codon in position 1552-1554. The polypeptide sequence according to the invention is shown in SEQ ID NO: 6.

Sequencing the complete cDNA fragment identified from clone PT0010072031R revealed a sequence 1526 base pairs in length and shown in SEQ ID NO: 11 with a start codon in position 92-94 and a stop codon in position 1400-1402. The polypeptide sequence according to the invention is shown in SEQ ID NO: 12.

Table. 2 shows the identities and homologies of desaturases according to the invention with each other and with the Physcomitrella patens and Mortierella alpina desaturase. The data were obtained with the aid of the Bestfit program under given parameters as defined hereinbelow as a subprogram of the following software: Wisconsin Package Version 10.0 (Genetics Computer Group (GCG), Madison, Wisc., USA). Henikoff, S. and Henikoff, J. G. (1992). Amino acid substitution matrices from protein blocks. Proc. Natl. Acad. Sci. USA 89: 10915-10919.

Furthermore, FIG. 5 shows the alignment of the Physcomitrella patens Δ6-acyl lipid desaturase with the polypeptide sequence of clone Pt_des6.

TABLE 2

| Homology/ | Search sequence | Search sequence |
|---|---|---|
| identity in % | Pp_des6 | Ma_des12 |
| Pt_des5 | 34.92/26.37 | n.d. |
| Pt_des6 | 50.69/41.06 | n.d. |
| Pt_des12 | n.d. | 48.58/38.92 |
| Pt_des12.2 | n.d. | 48.37/41.60 |

n.d. = not determined

With the aid of the algorithm TBLASTN 2.0.10: Altschul et al. 1997, “Gapped BLAST and PSI-BLAST: a new generation of protein database search programs”, Nucleic Acids Res. 25:3389-3402, sequences with the highest sequence homology or identity were identified via a local database alignment. The results are shown in Table 2A hereinbelow.

TABLE 2A
Homologs with the highest sequence homologies or
identities with polypeptide sequences according to
the invention of SEQ ID NO. 2, 4, 6 or 12

| Homo- | Search | Search | Search | Search |
|---|---|---|---|---|
| logy/iden- | sequence | sequence | sequence | sequence |
| tity (%) | PT001070010R | PT001072031R | PT001078032R | PT_des6 |
| L26296: | 50%/37% | n.d. | n.d. | n.d. |
| Fad2 A. | | | | |
| thaliana | | | | |
| U86072 | n.d. | 51/40 | n.d. | n.d. |
| Petro- | | | | |
| selinum | | | | |
| crispum | | | | |
| Fad2 | | | | |
| AL358652 | n.d. | n.d. | 45/30 | n.d. |
| L. major | | | | |
| putative | | | | |
| desaturase | | | | |
| AB020032 | n.d. | n.d. | n.d. | 53/38 |
| M. alpina | | | | |
| Δ6 | | | | |
| desaturase | | | | |

### Example 7

### Identification of Genes by Means of Hybridization

Gene Sequences can be Used for Identifying Homologous or Heterologous Genes from cDNA or Genomic Libraries.

Homologous genes (i.e. full-length cDNA clones which are homologous, or homologs) can be isolated via nucleic acid hybridization using, for example, cDNA libraries: the method can be used in particular for isolating functionally active full-length genes of those shown in SEQ ID NO: 3. Depending on the frequency of the gene of interest, 100 000 up to 1 000 000 recombinant bacteriophages are plated out and transferred to a nylon membrane. After denaturation with alkali, the DNA was immobilized on the membrane, for example by UV crosslinking. Hybridization is performed under high-stringency conditions. The hybridization and the wash steps are carried out in aqueous solution at an ionic strength of 1 M NaCl and a temperature of 68° C. Hybridization probes were generated for example by labeling with radioactive (³²P-) nick transcription (High Prime, Roche, Mannheim, Germany). The signals are detected by autoradiography.

Partially homologous or heterologous genes which are related but not identical can be identified analogously to the process described above using low-stringency hybridization and wash conditions. For the aqueous hybridization, the ionic strength was usually kept at 1 M NaCl, and the temperature was lowered gradually from 68 to 42° C.

The isolation of gene sequences which only exhibit homologies with an individual domain of, for example, 10 to 20 amino acids can be carried out using synthetic, radiolabeled oligonucleotide probes. Radiolabeled oligonucleotides are generated by phosphorylating the 5′ end of two complementary oligonucleotides with T4 polynucleotide kinase. The complementary oligonucleotides are hybridized and ligated to each other to give rise to concatemers. The double-stranded concatemers are radiolabeled for example by nick transcription. Hybridization is usually carried out under low-stringency conditions using high oligonucleotide concentrations.

Oligonucleotide hybridization solution:

6×SSC

0.01 M sodium phosphate

1 mM EDTA (pH 8)

0.5% SDS

100 μg/ml denatured salmon sperm DNA

0.1% dry low-fat milk

During the hybridization, the temperature is lowered stepwise to 5 to 10° C. below the calculated oligonucleotide Tm or down to room temperature (unless otherwise specified, RT=−23° C. in all experiments), followed by wash steps and autoradiography. Washing is carried out at extremely low stringency, for example three wash steps using 4×SSC. Further details are as described by Sambrook, J., et al. (1989), “Molecular Cloning: A Laboratory Manual”, Cold Spring Harbor Laboratory Press, or Ausubel, F. M., et al. (1994)“Current Protocols in Molecular Biology”, John Wiley & Sons.

### Example 8

### Identification of Target Genes by Screening Expression Libraries with Antibodies

To generate recombinant protein, for example E. coli, cDNA sequences were used (for example Qiagen QIAexpress pQE system). The recombinant proteins were then affinity-purified, usually via Ni-NTA affinity chromatography (Qiagen). The recombinant proteins were then used for raising specific antibodies, for example using standard techniques for immunizing rabbits. The antibodies were then affinity-purified using an Ni-NTA column which is desaturated with recombinant antigen, as described by Gu et al., (1994) BioTechniques 17:257-262. The antibody can then be used for screening expression cDNA libraries by immunological screening (Sambrook, J., et al. (1989), “Molecular Cloning: A Laboratory Manual”, Cold Spring Harbor Laboratory Press, or Ausubel, F. M., et al. (1994) “Current Protocols in Molecular Biology”, John Wiley & Sons).

### Example 9

### Transformation of Agrobacterium

Agrobacterium-Mediated plant transformation can be effected for example using the Agrobacterium tumefaciens strain GV3101-(pMP90-) (Koncz and Schell, Mol. Gen. Genet. 204 (1986) 383-396) or LBΔ4404- (Clontech) or C58C1 pGV2260 (Deblaere et al 1984, Nucl. Acids Res. 13, 4777-4788). The transformation can be carried out by standard transformation techniques (Deblaere et al., 1984, IBID.).

### Example 10

### Plant Transformation

Agrobacterium-mediated plant transformation can be effected using standard transformation and regeneration techniques (Gelvin, Stanton B., Schilperoort, Robert A., Plant Molecular Biology Manual, 2nd Ed., Dordrecht: Kluwer Academic Publ., 1995, in Sect., Ringbuc Zentrale Signatur: BT11-P ISBN 0-7923-2731-4; Glick, Bernard R., Thompson, John E., Methods in Plant Molecular Biology and Biotechnology, Boca Raton: CRC Press, 1993, 360 pp., ISBN 0-8493-5164-2).

For example, oilseed rape can be transformed by means of cotyledon or hypocotyl transformation (Moloney et al., Plant Cell 8 (1989) 238-242; De Block et al., Plant Physiol. 91 (1989) 694-701). The use of antibiotics for the selection of agrobacteria and plants depends on the binary vector and the agrobacterial strain used for the transformation. The selection of oilseed rape is normally carried out using kanamycin as selectable plant marker.

Agrobacterium-mediated gene transfer in linseed (Linum usitatissimum) can be carried out for example using a technique described by Mlynarova et al. (1994) Plant Cell Report 13:282-285.

The transformation of soybean can be carried out for example using a technique described in EP-A-0 0424 047 (Pioneer Hi-Bred International) or in EP-A-0 0397 687, U.S. Pat. No. 5,376,543, U.S. Pat. No. 5,169,770 (University Toledo).

Plant transformation using particle bombardment, polyethylene glycol-mediated DNA uptake or via the silicon carbonate fiber technique is described, for example, by Freeling and Walbot “The maize handbook” (1993) ISBN 3-540-97826-7, Springer Verlag New York).

### Example 11

### Plasmids for Plant Transformation

Suitable binary vectors and transformation markers Binary vectors such as pBinAR (Hofgen and Willmitzer, Plant Science 66 (1990) 221-230) or pGPTV (Becker et al 1992, Plant Mol. Biol. 20:1195-1197) can be used for transforming plants. The binary vectors can be constructed by ligating the cDNA in sense or antisense orientation into T-DNA. 5′ of the cDNA, a plant promoter activates cDNA transcription. A polyadenylation sequence is located 3′ of the cDNA. The binary vectors can contain different marker genes. Thus, for example, resistance can be effected by expressing the nptII marker gene under the control of the ³⁵S or the nos promoter. In particular, the nptII marker gene encoding kanamycin resistance mediated by neomycin phosphotransferase can be exchanged for the herbicide-resistant form of an acetolactate synthase gene (AHAS or ALS). The ALS gene is described in Ott et al., J. Mol. Biol. 1996, 263:359-360. The use of the hygromycin resistance gene is also suitable for some plants. The v-ATPase-c1 promoter can be cloned into plasmid pBin19 or pGPTV and exploited for marker gene expression by cloning it before the coding region of the ALS gene. The v-ATPase-c1 promoter stated corresponds to a 1153 base pair fragment from Beta vulgaris (Plant Mol. Biol., 1999, 39:463-475). The nos promoter stated [lacuna] Both sulfonylureas and imidazolinones such as imazethapyr or sulfonylureas can be used as antimetabolites for selection. As an alternative, the nos promoter may also be used for expressing the marker gene.

### Example 12

### Determination of Suitable Promoters for the Expression in Linseed

Tissue-specific expression can be achieved using a tissue-specific promoter. For example, seed-specific expression can be achieved by cloning the DC3 or LeB4 or the USP promoter or the phaseolin promoter 5′ of the cDNA. Any other seed-specific promoter elements such as, for example, the napin or arcelin promoter (Goossens et al. 1999, Plant Phys. 120(4):1095-1103 and Gerhardt et al. 2000, Biochimica et Biophysica Acta 1490(1-2):87-98) may also be used. The CaMV 35S promoter or a v-ATPase-c1 promoter can be used for constitutive expression in all of the plant.

In order to determine the properties of the promoter and to identify its essential elements which account for its tissue specificity, it is necessary to place the promoter itself or various fragments of the latter in front of what is known as a reporter gene, which makes possible a determination of the expression activity. An example of a reporter gene which may be mentioned is the bacterial β-glucuronidase (GUS) (Jefferson et al., EMBO J. 1987, 6, 3901-3907). The β-glucuronidase activity can be determined in situ by means of a chromogenic substrate such as 5-bromo-4-chloro-3-indolyl-β-D-glucuronic acid in the form of activity staining (Jefferson, 1987, Plant Molecular Biology Reporter 5, 387-405). To study the tissue specificity, the plant tissue is cut, embedded, stained and analyzed as described (for example Baumlein H et al., 1991 Mol Gen Genet 225: 121-128).

Fluorimetric GUS assay (by the method of Montgomery et al., 1993)

This assay permits the quantitative determination of the GUS activity in the tissue studied. To carry out the quantitative activity determination, MUG (4-methylumbelliferyl-β-D-glucuronide) is used as substrate for β-glucuronidase and is cleaved into MU (methylumbelliferone) and glucuronic acid.

To carry out this assay, a protein extract of the desired tissue is first prepared and the substrate of GUS is then added thereto. The substrate can only be measured fluorimetrically after conversion by GUS. At various points in time, samples are taken and subsequently measured in the fluorimeter. This assay was carried out on linseed embryos at various age stages (21, 24 or 30 days after the onset of flowering, daf - days after flowering). To this end, in each case one embryo was ground into a powder in a 2 ml reaction vessel with the aid of a vibrating mill (Retsch MM 2000) in liquid nitrogen. After addition of 100 ml of EGL buffer, the mixture was spun for 10 minutes at 25° C. and 14 000×g. The supernatant was removed and spun a second time. Again, the supernatant was transferred into a fresh reaction vessel and kept on ice until further use. 25 ml of this protein extract were treated with 65 ml of EGL buffer (without DTT) and employed in the GUS assay. At this point in time, 10 ml of the substrate MUG (10 mM 4-methylumbelliferyl-β-D-glucuronide) were added, the mixture was vortexed, and, immediately, 30 ml were removed to act as zero value and treated with 470 ml of stop buffer (0.2 M Na₂CO₃). This procedure was repeated for all samples at an interval of 30 seconds. The samples taken were stored in a refrigerator until they were measured. The readings were obtained after 1 h and after 2 h. To carry out the measurement in the fluorimeter, a calibration series was established which contained concentrations of from 0.1 mM to 10 mM MU (4-methylumbelliferone). If the readings of the samples were outside these concentrations, less protein extract was employed (10 ml, 1 ml, 1 ml from a 1:10 dilution), and shorter intervals were measured (0 h, 30 min, 1 h). The measurement was carried out at an excitation of 365 nm and an emission of 445 nm in a Fluoroscan II apparatus (Labsystem). As an alternative, cleavage of the substrate can be monitored fluorimetrically under alkaline conditions (excitation at 365 nm, measurement of the emission at 455 nm; SpectroFluorimeter BMG Polarstar+) as described in Bustos M. M. et al., 1989 Plant Cell 1:839-853. All samples were subjected to a protein concentration determination by the method of Bradford (1976) to obtain findings on the promoter activity and promoter strength in various tissues and plants.

EGL buffer

| | | |
|---|---|---|
| 0.1 | M | KPO₄, pH 7.8 |
| 1 | mM | EDTA |
| 5% | | Glycerol |
| 1 | M | DTT |

Further examples which may be mentioned for reporter genes which can be used in a similar fashion are, for example, the green fluorescent protein (GFP) and its derivatives (C.Reichel et al. (1996) Proc.Natl.Acad.Sci.USA 93, 5888-5893 and J.Sheen et al., (1995) Plant Journal 8, 777-784) and various luciferases (A.Millar et al. (1992) Plant Mol. Biol. Reporter 10, 324-414). The corresponding detection methods are known to the skilled worker and described, for example, in the literature mentioned.

Examples of promoter-reporter gene constructs for the abovementioned promoters are given hereinbelow. Fragments of these promoters can be isolated with the aid of the polymerase chain reaction and, using flanking sequences, tailored as desired on the basis of synthetic oligonucleotides.

Examples of oligonucleotides which may be used are the following:

| | |
|---|---|
| LeB4 front: | GAAAGCTTCTCGAGTTATGCATTTCTT |
| LeB4 rear: | GGGTCTAGATCTGTGACTGTGATAG |
| DC3a front: | AGTGGATCCCCGAGCTAACCACAACT |
| DC3a rear: | ATAAGCTTTTTCTTTGCAGA |
| napin front: | GAAAGCTTCTAATATGATAAACTCTG |
| napin rear: | GGGTCTAGAAACACATACAAACATCAC |

The methods are known to the specialist worker and generally known from the literature.

In a first step, the promoter fragments are amplified via PCR, cut with suitable restriction enzymes and cloned into the above cassettes. For example, the LeB4(700) PCR fragment is cut with XbaI and HindIII and cloned into vector pGPTV into the HindIII and XbaI cleavage sites 5′ upstream of the GUS reporter gene. The PCR-amplified DC3 promoter fragments can be cut with BamHI and HindIII, subcloned into, for example, pBluescript (Stratagene) and then cloned into pGPTV into suitable cleavage sites upstream of the GUS reporter gene.

For example, a 1055 bp napin promoter fragment which has been PCR-amplified with the above primers can, following restriction with HindIII and XbaI, be cloned into pGPTV upstream of the GUS reporter gene. A similar construct might be a 1100 bp napin promoter fragment cloned 5′ upstream of a GUS reporter gene with intron followed 3′ by an nos terminator, in a pHL9000 vector (Hausmann & Topfer, 1999).

Using above-described or equivalent constructs following transformation into linseed cv. Flanders, the GUS activity in transgenic linseed embryos of various age stages which have been transformed with one of the following constructs: napin-GUS, 35S-GUS, LeB4-GUS, USP-GUS can be measured. The readings are means of one to five measurements with various amounts of protein. In each case three embryos were analyzed quantitatively per construct.

Quantitative analysis revealed large differences between the various seed-specific promoters. The Brassica napus napin promoter proved to be less active by two to three powers of ten than the two Vicia faba promoters (LeB4 and USP). GUS activity increased in the sequence napin, LeB4 and USP. The activity of the positive control 35S was between LeB4 and USP, whereas the negative control, the untransformed wild type (cultivar Flanders), showed virtually no activity. The table which follows shows the means of the activities at the individual age stages and in total for each construct.

Table 3.4: Overview of the mean GUS activities of linseed embroyos transformed with various GUS constructs. daf: days after beginning of flowering.

Readings with an * are based on one measurement only.

| | GUS activity | GUS activity | GUS activity | GUS activity | |
|---|---|---|---|---|---|
| GUS | [nmol/h/mg | [nmol/h/mg | [nmol/h/mg | [nmol/h/mg | % vs. |
| construct | protein] | protein] | protein] | protein] | 35S |
| | Mean 21 daf | Mean 24 daf | Mean 30 daf | Overall mean | |
| Without | 0.06 | 0.06 | *0.02 | 0.05 | 0.0007 |
| 35S | 649.00 | 913.00 | 639.00 | 734.00 | 100.00 |
| Napin | 7.70 | 5.70 | 3.70 | 5.70 | 0.80 |
| LeB4 | 1778.00 | 253.00 | 283.00 | 771.00 | 105.00 |
| USP | 2843.00 | 3770.00 | 2107.00 | 2907.00 | 396.00 |

### Example 13

### Plasmids for Plant Transformation

Plants may be transformed using binary vectors such as pBinAR (Hofgen and Willmitzer, Plant Science 66 (1990) 221-230) or pGPTV (Becker et al 1992, Plant Mol. Biol. 20:1195-1197) or derivatives thereof. The binary vectors may be constructed by ligating the cDNA in sense or antisense orientation into T-DNA. 5′ of the cDNA, a plant promoter activates cDNA transcription. A polyadenylation sequence is located 3′ of the cDNA. The binary vectors can contain different marker genes. In particular, the nptII marker gene, which encodes neomycin-phosphotransferase-mediated kanaymicin resistance, may be exchanged for the herbicide-resistant form of an acetolactate synthase gene (abbreviation: AHAS or ALS). The ALS gene is described in Ott et al., J. Mol. Biol. 1996, 263:359-360. The v-ATPase-c1 promoter can be cloned into plasmid pBin19 or pGPTV and used for expressing the marker gene by being cloned upstream of the ALS coding region. The promoter mentioned corresponds to a 1153 base pair fragment of Beta vulgaris (Plant Mol Biol, 1999, 39:463-475). Not only sulfonylureas, but also imidazolinones such as imazethapyr or sulfonylureas may be used as antimetabolites for selection purposes.

Tissue-specific expression can be achieved by using a tissue-specific promoter. For example, seed-specific expression can be achieved by cloning the DC3 or the LeB4 or the USP promoter or the phaseolin promoter 5′ of the cDNA. Any other seed-specific promoter element can also be used, such as, for example, the napin or arcelin promoter (Goossens et al. 1999, Plant Phys. 120(4):1095-1103 and Gerhardt et al. 2000, Biochimica et Biophysica Acta 1490(1-2):87-98). The CaMV ³⁵S promoter or a v-ATPase C1 promoter can be used for constitutive expression in intact plants.

In particular, genes encoding desaturases and elongases can be cloned into a binary vector one after the other by constructing several expression cassettes in order to imitate the metabolic pathway in plants.

Within an expression cassette, the protein to be expressed can be targeted into a cellular compartment using a signal peptide, for example for plastids, mitochondria or the endoplasmic reticulum (Kermode, Crit. Rev. Plant Sci. 15, 4 (1996) 285-423). The signal peptide is cloned 5′ in-frame with the cDNA in order to achieve subcellular localization of the fusion protein.

Examples of multiexpression cassettes are given hereinbelow.

I.) Promoter-Terminator Cassettes

Expression cassettes are composed of at least two functional units, such as a promoter and a terminator. Further desired gene sequences such as targeting sequences, coding regions of genes or parts thereof etc. can be inserted between promoter and terminator. In order to construct expression cassettes, promoters and terminators (USP promoter: Baeumlein et al., Mol. Gen. Genet., 1991, 225 (3):459-67); OCS terminator: Gielen et al. EMBO J. 3 (1984) 835 et seq.) are isolated with the aid of polymerase chain reaction and tailor-made as desired with flanking sequences based on synthetic oligonucleotides.

Examples of the oligonucleotides which can be used are the following:

| | |
|---|---|
| USP1 front: | CCGGAATTCGGCGCGCCGAGCTCCTCGAGCAAATTTACACATTGCCA |
| USP2 front: | CCGGAATTCGGCGCGCCGAGCTCCTCGAGCAAATTTACACATTGCCA |
| USP3 front: | CCGGAATTCGGCGCGCCGAGCTCCTCGAGCAAATTTACACATTGCCA |
| USP1 back: | AAAACTGCAGGCGGCCGCCCACCGCGGTGGGCTGGCTATGAAGAAATT |
| USP2 back: | CGCGGATCCGCTGGCTATGAAGAAATT |
| USP3 back: | TCCCCCGGGATCGATGCCGGCAGATCTGCTGGCTATGAAGAAATT |
| OCS1 front: | AAAACTGCAGTCTAGAAGGCCTCCTGCTTTAATGAGATAT |
| OCS2 front: | CGCGGATCCGATATCGGGCCCGCTAGCGTTAACCCTGCTTTAATGAGATAT |
| OCS3 front: | TCCCCCGGGCCATGGCCTGCTTTAATGAGATAT |
| OCS1 back: | CCCAAGCTTGGCGCGCCGAGCTCGAATTCGTCGACGGACAATCAGTAAATTGA |
| OCS2 back: | CCCAAGCTTGGCGCGCCGAGCTCGAATTCGTCGACGGACAATCAGTAAATTGA |
| OCS3 back: | CCCAAGCTTGGCGCGCCGAGCTCGTCGACGGACAATCAGTAAATTGA |

The methods are known to the skilled worker in the art and are generally known from the literature.

In a first step, a promoter and a terminator are amplified via PCR. Then, the terminator is cloned into a recipient plasmid and, in a second step, the promoter is inserted before the terminator. This gives an expression cassette on a carrier plasmid. Plasmids pUT1, pUT2 and pUT3 are generated on the basis of plasmid pUC19.

The constructs are defined in accordance with the invention in SEQ ID NO: 13, 14 and 15. Based on pUC19, they comprise the USP promoter and the OCS terminator. Based on these plasmids, construct pUT12 is generated by cutting pUT1 with SalI/ScaI and cutting pUT2 with XhoI/ScaI. The fragments in the expression cassette are ligated and transformed into E. coli XLI blue MRF. After singling out ampicillin-resistant colonies, DNA is prepared, and those clones which comprise two expression cassettes are identified by restriction analysis. The XhoI/SalI ligation of compatible ends has eliminated the two cleavage sites XhoI and SalI between the expression cassettes. This gives rise to plasmid pUT12, which is defined in SEQ ID NO: 16. pUT12 is subsequently cut again with SalI/ScaI and pUT3 with XhoI/ScaI. The fragments comprising the expression cassettes are ligated and transformed into E. coli XLI blue MRF. After singling out ampicillin-resistant columns, DNA is prepared, and those clones which comprise three expression cassettes are identified by restriction analysis. In this manner, a set of multiexpression cassettes is created which can be exploited for inserting the desired DNA and is described in Table 3 and can additionally incorporate further expression cassettes.

They comprise the following elements:

TABLE 3

| | Cleavage sites | Multiple | Cleavage sites |
|---|---|---|---|
| pUC19 | for the USP | cloning cleavage | behind the OCS |
| derivate | promoter | sites | terminator |
| pUT1 | EcoRI/AscI/ | BstXI/NotI/ | SalI/EcoRI/ |
| | SacI/XhoI | PstI/XbaI/StuI | SacI/AscI/ |
| | | | HindIII |
| pUT2 | EcoRI/AscI/ | BamHI/EcoRV/ | SalI/EcoRI/ |
| | SacI/XhoI | ApaI/NheI/HpaI | SacI/AscI/ |
| | | | HindIII |
| pUT3 | EcoRI/AscI/ | BglII/NaeI/ | SalI/SacI/ |
| | SacI/XhoI | ClaI/SmaI/NcoI | AscI/HindIII |
| pUT12 | EcoRI/ASCI/ | BstXI/NotI/ | SalI/EcoRI/ |
| Double | SacI/XhoI | PstI/XbaI/StuI | SalI/AscI/ |
| expression | | and | HindIII |
| cassette | | BamHI/EcoRV/ | |
| | | ApaI/NheI/HpaI | |
| pUT123 | EcoRI/AscI/ | 1. BstXI/NotI/ | SalI/SacI/AscI/ |
| Triple | SacI/XhoI | PstI/XbaI/StuI | HindIII |
| expression | | and | |
| cassette | | 2. BamHI/EcoRV/ | |
| | | ApaI/NheI/HpaI | |
| | | and | |
| | | 3. BglII/NaeI/ | |
| | | ClaI/SmaI/NcoI | |

Furthermore, further multiexpression cassettes can be generated and employed for the seed-specific gene expression, as described and as specified in greater detail in Table 4, with the aid of the

i) USP promoter or with the aid of the

ii) approx. 700 base pair 3′ fragment of the LeB4 promoter or with the aid of the

iii) DC3 promoter.

The DC3 promoter is described in Thomas, Plant Cell 1996, 263:359-368 and consists merely of the region -117 to +27, which is why it therefore constitutes one of the smallest known seed-specific promoters.

Fragments can be isolated from these promoters with the aid of the polymerase chain reaction and, using flanking sequences, tailored as desired on the basis of synthetic oligonucleotides.

Examples of oligonucleotides which can be used are the following:

| | |
|---|---|
| LeB4 front: | GAAAGCTTCTCGAGTTATGCATTTCTT |
| LeB4 rear: | GGGTCTAGATCTGTGACTGTGATAG |
| DC3a front: | CCGGAATTCGGCGCGCCGAGCTCCTCGAG |
| DC3a rear: | CGCGGATCCTAGCTTTTTCTTGGCAGATG |

The methods are known to the specialist worker and generally known from the literature.

In a first step, the promoter fragments are amplified via PCR, cut with suitable restriction enzymes and cloned into the above cassettes. For example, the LeB4 (700) PCR fragment is cut with XhoI and BglII and inserted into the XHOI and BglII cleavage sites of plasmid pUT3 to give rise to pLT3.

Advantageous expression cassettes comprise, based on pUC19 (Vieira and Messing (1982); Gene 19, 259), SEQ ID NO: 32, the LeB4 promoter and the sequences SEQ ID NO: 33, SEQ ID NO: 34 or SEQ ID NO: 35. Based on these plasmids, construct pLT12 is generated by cleaving PUTI by means of SalI/ScaI and cleaving pUT2 by means of XhoI/ScaI. The fragments comprising the expression cassettes are ligated and transformed into E. coli XLI blue MRF. After singling out ampicillin-resistant colonies, DNA is prepared, and those clones which contain two expression cassettes are identified by restriction analysis. The XhoI/SalI ligation of compatible ends has resulted in eliminating the two cleavage sites XhoI and SalI between the expression cassettes. This results in plasmid pUT12, which is defined in SEQ ID NO: 16. Subsequently, pUT12, in turn, is cleaved by means of Sal/ScaI and pUT3 is cleaved by means of XhoI/ScaI. The fragments comprising the expression cassettes are ligated and transformed into E. coli XLI blue MRF. After singling out ampicillin-resistant colonies, DNA is prepared, and those clones containing three expression cassettes are identified by restriction analysis. In this fashion, a selection of multi-expression cassettes is created which can be used for inserting the desired DNA, is described in Table 3 and can additionally incorporate further expression cassettes.

The expression cassettes can comprise several copies of the same promoter or else be constructed via three different promoters.

TABLE 4
Multiple expression cassettes

| | Cleavage sites | Multiple | Cleavage |
|---|---|---|---|
| Plasmid name of | before the | cloning | sites behind |
| the pUC19 | respective | cleavage | the OCS |
| derivative | promoter | sites | terminator |
| pUT1 | EcoRI/AscI/SacI/ | (1) BstXI/NotI/ | SalI/EcoRI/ |
| (pUC19 with | XhoI | PstI/XbaI/StuI | SacI/AscI/ |
| USP-OCS1) | | | HindIII |
| pDCT | EcoRI/AscI/SacI/ | (2) BamHI/ | SalI/EcoRI/ |
| (pUC19 with | XhoI | EcoRV/ApaI/ | SacI/AscI/ |
| DC3-OCS) | | NheI/HpaI | HindIII |
| pLeBT | EcoRI/AscI/SacI/ | (3) BglII/NaeI/ | SalI/SacI/ |
| (pUC19-with | XhoI | ClaI/SmaI/NcoI | AscI/HindIII |
| LeB4(700)-OCS) | | | |
| pUD12 | EcoRI/AscI/SacI/ | (1) BstXI/NotI/ | SalI/EcoRI/ |
| (pUC19 with | XhoI | PstI/XbaI/StuI | SacI/AscI/ |
| USP-OCS1 and | | and | HindIII |
| with DC3-OCS) | | (2) BamHI/ | |
| | | EcoRV/ApaI/ | |
| | | NheI/HpaI | |
| pUDL123 | EcoRI/AscI/SacI/ | (1) BstXI/NotI/ | SalI/SacI/ |
| Triple expression | XhoI | PstI/XbaI/StuI | AscI/HindIII |
| cassette | | and | |
| (pUC19 with | | (2) BamHI/ | |
| USP/DC3 and | | (EcoRV*)/ApaI/ | |
| LeB4-700) | | NheI/HpaI | |
| | | and | |
| | | (3) BglII/NaeI/ | |
| | | ClaI/SmaI/NcoI | |

*EcoRV cleavage site in the 700 base-pair fragment of the LeB4 promoter (LeB4-700)

Further promoters for multi-gene constructs can be generated analogously, in particular using the

a) 2.4 kb fragment of the LeB4 promoter (Baumlein et al., 1991: Mol.Gen.Genet. 225, 121-128) or with the aid of the

b) phaseolin promoter (Bustos et al. (1989) Plant Cell 1,839-853) or with the aid of the

c) constitutive v-ATPase c1 promoter.

It may be particularly desirable to use further especially suitable promoters for constructing seed-specific multi-expression cassettes such as, for example, one of the fragments of the napin promoter (Stalberg et al., 1993: Plant Mol. Biol.23,671-683) or the arcelin-5 promoter (A.Goossens et al., 1999: plant Physiol. 120,1095-1104).

ii) Generation of expression construct in pUC19 derivatives or pGPTV derivatives receiving promoter and terminator and comprised in combination with desired gene sequences for PUFA gene expression in plant expression cassettes.

Using AscI, multi-expression cassettes can be inserted directly from pUC19 derivatives of Table 3 into the vector pGPTV+AscI (see iii.)) via the AscI cleavage site and are available for inserting target genes. The gene constructs in question (pBUT1 is shown in SEQUENCE ID NO: 20, pBUT2 is shown in SEQUENCE ID NO: 21, pBUT3 is shown in SEQUENCE ID NO: 22, PBUT 12 is shown in SEQUENCE ID NO: 22 and pBUT123 is shown in SEQUENCE ID NO: 24) are available in accordance with the invention in kit form.

As an alternative, gene sequences can be inserted into the pUC19-based expression cassettes and inserted into pGPTV+AscI in the form of an AscI fragment.

In pUT12, the Δ6-elongase Pp_PSE1 is first inserted into the first cassette via BstXI and XbaI. Then, the moss Δ6-desaturase (Pp_des6) is inserted into the second cassette via BamHI/NaeI. This gives rise to the construct pUT-ED. The AscI fragment from plasmid pUT-ED is inserted into the AscI-cut vector pGPTV+AscI, and the orientation of the inserted fragment is determined by restriction or sequencing. This gives rise to plasmid pB-DHGLA, whose complete sequence is shown in SEQUENCE ID NO. 25. The coding region of the Physcomitrella Δ6-elongase is shown in SEQUENCE ID NO. 26, that of the Physcomitrella Δ6-desaturase in SEQUENCE ID NO: 27.

In pUT123, the Δ6-elongase Pp_PSE1 is first inserted into the first cassette via BstXI and XbaI. Then, the moss Δ6-desaturase (Pp_des6) is inverted into the second cassette via BamHI/NaeI, and, finally, the Phaeodactylum Δ5-desaturase (Pt_des5) is inserted into the third cassette via BglII. The triple construct is given the name pARA1. Taking into consideration sequence-specific restriction cleavage sites, further expression cassettes termed pARA2, pARA3 and pARA4 can be generated, as shown in Table 5.

The AscI fragment from plasmid pARA1 is inserted into the AscI-cut vector pGPTV+AscI and the orientation of the inserted fragment is determined by means of restriction or sequencing. The complete sequence of the resulting plasmid pBARA1 is shown in SEQUENCE ID NO. 28. The coding region of the Physcomitrella Δ6-elongase is shown in SEQUENCE ID NO. 29, that of the Physcomitrella Δ6-desaturase in SEQUENCE ID NO: 30 and that of the Phaeodactylum tricornutum Δ5-desaturase in SEQUENCE ID NO: 31.

TABLE 5
Combinations of desaturases and elongases

| | Gene | | | |
|---|---|---|---|---|
| | Plasmid | Δ6-desaturase | Δ5-desaturase | Δ6-elongase |
| 1 | PUT-ED | Pp_des6 | — | Pp_PSE1 |
| 2 | pARA1 | Pt_des6 | Pt_des5 | Pp_PSE1 |
| 3 | pARA2 | Pt_des6 | Ce_des5 | Pp_PSE1 |
| 4 | pARA3 | Pt_des6 | Ce_des5 | Pp_PSE1 |
| 5 | pARA4 | Ce_des6 | Ce_des5 | Ce_PSE1 |
| 6 | PBDHGLA | Pt_des6 | — | Pp_PSE1 |
| 7 | PEARAI | Pt_des6 | Pt_des5 | Pp_PSE1 |

Plasmids 1 to 5 are pUC derivatives, plasmids 6 to 7 are binary plant transformation vectors

Pp=Physcomitrella patens, Pt=Phaeodactylum tricornutum

Pp_PSE1 corresponds to the sequence of SEQ ID NO: 9.

PSE=PUFA-specific Δ6-elongase

Ce_des5=Caenorhabditis elegans A5-desaturase (Genbank Acc. No. AF078796)

Ce_des6=Caenorhabditis elegans A6-desaturase (Genbank Acc. No. AF031477, bases 11-1342)

Ce_PSE1=Caenorhabditis elegans A6-elongase (Genbank Acc. No. AF244356, bases 1-867)

Further desaturases or elongase gene sequences can also be inserted into expression cassettes of the above-described type, such as, for example, Genbank Acc. No. AF231981, NM_{—}013402, AF206662, AF268031, AF226273, AF110510 or AF110509.

iii) Transfer of Expression Cassettes into Vectors for the Transformation of Agrobacterium tumefaciens and for the Transformation of Plants

Chimeric gene constructs based on those described in pUC19 can be inserted into the binary vector pGPTV by means of AscI. For this purpose, the multiple cloning sequence is extended by an AscI cleavage site. For this purpose, the polylinker is newly synthesized as two double-stranded oligonucleotides, an additional AscI DNA sequence being inserted. The oligonucleotide is inserted into the vector pGPTV by means of EcoRI and HindIII. This gives rise to plasmid pGPTV+AscI. The cloning techniques required are known to the skilled worker and can simply be found as described in Example 1.

### Example 14

### In-Vivo Mutagenesis

The in-vivo mutagenesis of microorganisms can be performed by passaging the plasmid DNA (or any other vector DNA) via E. coli or other microorganisms (for example Bacillus spp. or yeasts such as Saccharomyces cerevisiae) in which the ability of maintaining the integrity of the genetic information is disrupted. Conventional mutator strains have mutations in the genes for the DNA repair system (for example mutHLS, mutD, mutT and the like; as reference, see Rupp, W. D. (1996) DNA repair mechanisms, in: Escherichia coli and Salmonella, pp. 2277-2294, ASM: Washington). These strains are known to the skilled worker. The use of these strains is illustrated for example in Greener, A., and Callahan, M. (1994) Strategies 7:32-34. The transfer of mutated DNA molecules into plants is preferably effected after the microorganisms have been selected and tested. Transgenic plants are generated in accordance with various examples in the examples section of the present document.

### Example 15

### Studying the Expression of a Recombinant Gene Product in a Transformed Organism

The activity of a recombinant gene product in the transformed host organism can be measured at the transcription and/or translation level.

A suitable method for determining the amount of transcription of the gene (which indicates the amount of RNA available for translation of the gene product) is to carry out a Northern blot as specified hereinbelow (for reference, see Ausubel et al. (1988) Current Protocols in Molecular Biology, Wiley: New York, or the abovementioned examples section) in which a primer which is designed such that it binds to the gene of interest is labeled with a detectable label (usually radioactivity or chemiluminescent) so that, when the total RNA of a culture of the organism is extracted, separated on a gel, transferred to a stable matrix and incubated with this probe, binding and the extent of binding of the probe indicate the presence and also the quantity of the mRNA for this gene. This information indicates the degree of transcription of the transformed gene. The total cell RNA can be prepared from cells, tissues or organs by a plurality of methods, all of which are known in the art, such as, for example, the method of Bormann, E. R., et al. (1992) Mol. Microbiol. 6:317-326.

Northern Hybridization

For the RNA hybridization, 20 μg of total RNA or 1 μg of poly(A)⁺ RNA were separated by gel electrophoresis in 1.25% strength agarose gel using formaldehyde as described by Amasino (1986, Anal. Biochem. 152, 304), transferred to positively charged nylon membranes (Hybond N+, Amersham, Braunschweig) by capillary attraction using 10×SSC, immobilized by means of UV light and prehybridized for 3 hours at 68° C. using hybridization buffer (10% dextran sulfate w/v, 1 M NaCl, 1% SDS, 100 mg herring sperm DNA). The DNA probe had been labeled with the Highprime DNA labeling kit (Roche, Mannheim, Germany) during the prehybridization stage using alpha-³²P-dCTP (Amersham, Braunschweig, Germany). The hybridization was carried out after adding the labeled DNA probe in the same buffer at 68° C. overnight. The wash steps were carried out twice for 15 minutes using 2×SSC and twice for 30 minutes using 1×SSC, 1% SDS, at 680C. The sealed filters were exposed at −70° C. for a period of 1 to 14 days.

Standard techniques such as a Western blot (see, for example, Ausubel et al. (1988) Current Protocols in Molecular Biology, Wiley: New York) can be employed for studing the presence or the relative quantity of protein translated from this mRNA. In this method, the total cell proteins are extracted, separated by means of gel electrophoresis, transferred to a matrix such as nitrocellulose, and incubated with a probe such as an antibody which specifically binds to the desired protein. This probe is usually provided with a chemiluminescent or calorimetric label which can be detected readily. The presence and the quantity of the label observed indictes the presence and quantity of the desired mutated protein which is present in the cell.

### Example 16

### Analysis of the Effect of the Recombinant Proteins on the Production of the Desired Product

The effect of the genetic modification in plants, fungi, algae, ciliates or on the production of a desired compound (such as a fatty acid) can be determined by growing the modified microorganisms or the modified plant under suitable conditions (such as those described above) and analyzing the medium and/or the cell components for the increased production of the desired product (i.e. of lipids or a fatty acid). These analytical techniques are known to the skilled worker and encompass spectroscopy, thin-layer chromatography, various staining methods, enzymatic and microbiological methods, and analytical chromatography such as high-performance liquid chromatography (see, for example, Ullman, Encyclopedia of Industrial Chemistry, Vol. A2, pp. 89-90 and pp. 443-613, VCH Weinheim (1985); Fallon, A., et al., (1987) “Applications of HPLC in Biochemistry” in: Laboratory Techniques in Biochemistry and Molecular Biology, Vol. 17; Rehm et al. (1993) Biotechnology, Vol. 3, Chapter III: “Product recovery and purification”, pp. 469-714, VCH Weinheim; Belter, P. A., et al. (1988) Bioseparations: downstream processing for Biotechnology, John Wiley and Sons; Kennedy, J.F., and Cabral, J. M. S. (1992) Recovery processes for biological Materials, John Wiley and Sons; Shaeiwitz, J. A., and Henry, J. D. (1988) Biochemical Separations, in: Ullmann's Encyclopedia of Industrial Chemistry, Vol. B3; Chapter 11, pp. 1-27, VCH Weinheim; and Dechow, F. J. (1989) Separation and purification techniques in biotechnology, Noyes Publications).

In addition to the abovementioned methods, plant lipids are extracted from plant materials as described by Cahoon et al. (1999) Proc. Natl. Acad. Sci. USA 96 (22):12935-12940, and Browse et al. (1986) Analytic Biochemistry 152:141-145. Qualitative and quantitative lipid or fatty acid analysis is described in Christie, William W., Advances in Lipid Methodology, Ayr/Scotland: Oily Press (Oily Press Lipid Library; 2); Christie, William W., Gas Chromatography and Lipids. A Practical Guide—Ayr, Scotland: Oily Press, 1989, Repr. 1992, IX, 307 S. (Oily Press Lipid Library; 1); “Progress in Lipid Research, Oxford: Pergamon Press, 1 (1952)-16 (1977) under the title: Progress in the Chemistry of Fats and Other Lipids CODEN.

In addition to measuring the end product of the fermentation, it is also possible to analyze other components of the metabolic pathways which are used for producing the desired compound, such as intermediates and byproducts, in order to determine the overall production efficiency of the compound. The analytical methods encompass measurements of the nutrient quantities in the medium (for example sugars, hydrocarbons, nitrogen sources, phosphate and other ions), measurements of biomass concentration and growth, analysis of the production of customary metabolites of biosynthetic pathways, and measurements of gases which are generated during fermentation. Standard methods for these measurements are described in Applied Microbial Physiology; A Practical Approach, P. M. Rhodes and P. F. Stanbury, Ed., IRL Press, S. 103-129; 131-163 and 165-192 (ISBN: 0199635773) and references cited therein.

One example is the analysis of fatty acids (abbreviations: FAME, fatty acid methyl ester; GC-MS, gas-liquid chromatography/mass spectrometry; TAG, triacylglycerol; TLC, thin-layer chromatography).

The unambiguous detection of the presence of fatty acid products can be obtained by analyzing recombinant organisms by analytical standard methods: GC, GC-MS or TLC, as they are described on several occasions by Christie and the references therein (1997, in: Advances on Lipid Methodology, Fourth Edition: Christie, Oily Press, Dundee, 119-169; 1998, gas chromatography/mass spectrometry methods, Lipide 33:343-353).

The material to be analyzed can be disrupted by ultrasonication, grinding in a glass mill, liquid nitrogen and grinding or by other applicable methods. After disruption, the material must be centrifuged. The sediment is resuspended in distilled water, heated for 10 minutes at 100° C., ice-cooled and recentrifuged, followed by extraction in 0.5 M sulfuric acid in methanol with 2% dimethoxypropane for 1 hour at 90° C., which leads to hydrolyzed oil and lipid compounds which give transmethylated lipids. These fatty acid methyl esters are extracted in petroleum ether and finally subjected to GC analysis using a capillary column (Chrompack, WCOT Fused Silica, CP-Wax-52 CB, 25 μm, 0.32 mm) at a temperature gradient between 170° C. and 240° C. for 20 minutes and 5 minutes at 240° C. The identity of the resulting fatty acid methyl esters must be defined using standards which are commercially available (i.e. Sigma).

In the case of fatty acids for which no standards are available, the identity must be demonstrated via derivatization followed by GC/MS analysis. For example, the localization of fatty acids with triple bonds must be demonstrated via GC/MS following derivatization with 4,4-dimethoxyoxazolin derivatives (Christie, 1998, see above).

Expression Constructs in Heterologous Microbial Systems

Strains, Wash Conditions and Plasmids

The Escherichia coli strain XL1 Blue MRF′ kan (Stratagene) was used for subcloning novel Physcomitrella patens desaturase pPDesaturasel. For functionally expressing this gene, we used the Saccharomyces cerevisiae strain INVSc 1 (Invitrogen Co.). E. coli was grown in Luria-Bertini broth (LB, Duchefa, Haarlem, the Netherlands) at 37° C. If necessary, ampicillin (100 mg/liter) was added, and 1.5% of agar (w/v) was added for solid LB media. S. cerevisiae was grown at 30° C. either in YPG medium or in complete minimal dropout uracil medium (CMdum; see in: Ausubel, F. M., Brent, R., Kingston, R. E., Moore, D. D., Seidman, J. G., Smith, J. A., Struhl, K., Albright, L. B., Coen, D. M., and Varki, A. (1995) Current Protocols in Molecular Biology, John Wiley & Sons, New York) together with 2% (w/v) of either raffinose or glucose. For solid media, 2% (w/v) of Bacto™-agar (Difco) were added. The plasmids used for cloning an expression are pUC18 (Pharmacia) and pYES2 (Invitrogen Co.).

### Example 17

### Cloning and Expression of PUFA-Specific Phaeodactylum tricornutum Desaturases

For the expression in yeast, the Phaeodactylum tricornutum cDNA clones from SEQ ID NO: 1, 3, 5 or 11 or the sequences from SEQ ID NO: 7 or 9 or other desired sequences were first modified in such a way that only the coding regions are amplified by means of polymerase chain reaction with the aid of two oligonucleotides. Care was taken that a consensus sequence for the start codon was retained for efficient translation. To this end, either the base sequence ATA or AAA was selected and inserted into the sequence before the ATG (Kozak, M. (1986) Point mutations define a sequence flanking the AUG initiator codon that modulates translation by eukaryotic ribosomes, Cell 44, 283-292). A restriction cleavage site was additionally introduced before this consensus triplet, which restriction cleavage site must be compatible with the cleavage site of the target vector into which the fragment is to be cloned and with the aid of which gene expression is to take place in microorganisms or plants.

The PCR reaction was carried out with plasmid DNA as template in a thermocycler (Biometra) using Pfu DNA (Stratagene) polymerase and the following temperature program: 3 minutes at 96° C., followed by 30 cycles with 30 seconds at 96° C., 30 seconds at 55° C. and 2 minutes at 72° C., 1 cycle with 10 minutes at 72° C. and stop at 4° C. The annealing temperature was varied depending on the oligonucleotides chosen. A synthesis time of approximately one minute can be assumed per kilobase pairs of DNA. Further parameters which have an effect on the PCR, such as, for example, Mg ions, salt, DNA polymerase and the like are known to the skilled worker and can be varied as required.

The correct size of the amplified DNA fragment was controlled by means of agarose TBE gel electrophoresis. The amplified DNA was extracted from the gel using the QIAquick gel extraction kit (QIAGEN) and ligated into the SmaI restriction site of the dephosphorylated vector pUC18 using the Sure Clone Ligations Kit (Pharmacia), giving rise to the pUC derivatives. Following the transformation of E. coli XL1 Blue MRF′ kan, a DNA mini preparation (Riggs, M. G., & McLachlan, A. (1986) A simplified screening procedure for large numbers of plasmid mini-preparation. BioTechniques 4, 310-313) was carried out on ampicillin-resistant transformants, and positive clones were identified by means of BamHI restriction analysis. The sequence of the cloned PCR product was confirmed by resequencing using the ABI PRISM Big Dye Terminator Cycle Sequencing Ready Reaction Kit (Perkin-Elmer, Weiterstadt).

Δ5 acyl lipid desaturase, Pt_des5

| | |
|---|---|
| Primer | GAG CTC ACA TAA TGG CTC CGG ATG CGG ATA AGC |
| 1 | |
| Primer | CTC GAG TTA CGC CCG TCC GGT CAA GGG |
| 2 | |

The PCR fragment (1428 bp) was cloned into pUC18 with the aid of the Sure Clone kit (Pharmacia), the inserted fragment was digested with SacI/XhoI, and the fragment was inserted into pYES2 or pYES6 with the aid of suitable restriction cleavage sites.

Δ6 acyl lipid desaturase, Pt_des6
Primer 3
GGA TCC ACA TAA TGG GCA AAG GAG GGG ACG CTC GGG
Primer 4
CTC GAG TTA CAT GGC GGG TCC ATC GGG

The PCR fragment (1451 bp) was cloned into pUC18 with the aid of the Sure Clone kit (Pharmacia), the inserted fragment was digested with BamHI/XhoI, and the fragment was inserted into pYES2 or pYES6 with the aid of suitable restriction cleavage sites.

Δ12 acyl lipid desaturase, Pt_des12

| | |
|---|---|
| Primer 5 | GGA TCC ACA TAA TGG TTC GCT TTT CAA CAG CC |
| Primer 6 | CTC GAG TTA TTC GCT CGA TAA TTT GC |

Δ12 acyl lipid desaturase, Pt_des12.2

| | |
|---|---|
| Primer 7 | GGA TCC ACA TAA TGG GTA AGG GAG GTC AAC G |
| Primer 8 | CTC GAG TCA TGC GGC TTT GTT TCG C |

The PCR fragment (1505 bp) was cloned into pUC18 with the aid of the Sure Clone kit (Pharmacia), the inserted fragment was digested with BamHI/XhoI, and the fragment was inserted into pYES2 or pYES6 with the aid of suitable restriction cleavage sites.

The plasmid DNA was cleaved with restriction enzyme(s) to match the introduced cleavage site of the primer sequence, and the fragment obtained was ligated into the compatible restriction sites of the dephosphorylated yeast/E. coli shuttle vector pYES2 or pYES6, giving rise to pYES derivatives. Following the transformation of E. coli, and DNA minipreparation from the transformants, the orientation of the DNA fragment in the vector was verified by suitable restriction cleavage or sequencing. One clone was used for the DNA maxipreparation with the Nucleobond® AX 500 plasmid DNA extraction kit (Macherey-Nagel, Duringen).

Saccharomyces cerevisiae INVSc1 was transformed with the pYES derivatives and pYES blank vector by means of a PEG/lithium acetate protocol (Ausubel et al., 1995). Following selection on CMdum agar plates with 2% glucose, pYES derivative transformants and one pYES2 transformant were selected for further cultivation and functional expression. For pYES6 derivatives, blasticidin was used as antimetabolite. In the case of coexpression based on pYES2 and pYES6, selection was carried out with blasticidin on minimal medium.

Functional Expression of a Desaturase Activity in Yeast

Preculture

20 ml of liquid CMdum dropout uracil medium which, however, contains 2% (w/v) of raffinose were inoculated with the transgenic yeast clones (pYES2) and grown for 3 days at 30° C., 200 rpm, until an optical density at 600 nm (OD₆₀₀) of 1.5 to 2 had been reached. If pYES6 was used as vector, there was additional selection on blasticidin as antimetabolite.

Main Culture

For expression, 20 ml of liquid CMdum dropout uracil medium which, however, contains 2% of raffinose and 1% (v/v) of Tergitol NP-40 were supplemented with fatty acid substrates to a final concentration of 0.003% (w/v). The media were inoculated with the precultures to an OD₆₀₀ of 0.05. Expression was induced for 16 hours at an OD₆₀₀ of 0.2, using 2% (w/v) of galactose, whereupon the cultures were harvested at an OD₆₀₀ of 0.8-1.2.

Fatty Acid Analysis

The total fatty acids were extracted from yeast cultures and analyzed by means of gas chromatography. To this end, cells of 5 ml of culture were harvested by centrifugation (1 000×g, 10 minutes, 4° C.) and washed once with 100 mM NaHCO₃, pH 8.0 in order to remove residual medium and fatty acids. To prepare the fatty acid methyl ester(s) (FAMEs or, in the singular, FAME), the cell sediments were treated for 1 hour at 80° C. with 1 M methanolic H₂SO₄ and 2% (v/v) dimethoxypropane. The FAMEs were extracted twice with 2 ml of petroleum ether, washed once with 100 mM NaHCO₃, pH 8.0, and once with distilled water, and dried with Na₂SO₄. The organic solvent was evaporated under a stream of argon, and the FAMEs were dissolved in 50 μl of petroleum ether. The samples were separated on a ZEBRON-ZB Wax capillary column (30 m, 0.32 mm, 0.25 μm; Phenomenex) in a Hewlett Packard 6850 gas chromatograph equipped with flame ionization detector. The oven temperature was programmed from 70° C. (hold for 1 minute) to 200° C. at a rate of 20° C./minute, then to 250° C. (hold for 5 minutes) at a rate of 5° C./minute and finally to 260° C. at a rate of 5° C./minute. Nitrogen was used as the carrier gas (4.5 ml/minute at 70° C.). The fatty acids were identified by comparison with retention times of FAME standards (SIGMA).

Expression Analysis

The ratios of the fatty acid substrates which had been added and taken up were determined, thus recording the quantity and quality of the desaturase reaction in accordance with Table 6, Table 7 and Table 8.

The result of the expression of a Phaeodactylum tricornutum A6-acyl lipid desaturase in yeast:

TABLE 6

pYES2-Ptd6 fed with

| Fatty acid | pYES2 | — | +18:2 | +18:3 |
|---|---|---|---|---|
| 16:0 | 13.3 | 18.9 | 28.4 | 16.7 |
| 16:1Δ9 | 45.4 | 44.7 | 12.5 | 16.9 |
| 16:2Δ6, 9 | — | 4.3 | — | — |
| 18:0 | 4.9 | 6.3 | 10.4 | 9.1 |
| 18:1Δ9 | 36.4 | 24.2 | 6.8 | 11.8 |
| 18:2Δ6, 9 | — | 1.8 | — | — |
| 18:2Δ9, 12 | — | — | 33.4 | — |
| 18:3Δ6, 12, 15 | — | — | 4.9 | — |
| 18:3Δ9, 12, 15 | — | — | | 43.1 |
| 18:4Δ6, 9, 12, 15 | — | — | — | 2.3 |

The data represent mol % of corresponding cis-fatty acids.

Result of the expression of a Phaeodactylum tricornutum Δ5-acyl lipid desaturase in yeast:

TABLE 7

| | | | pYES_PtD5-construct fed with | | | | | | |
|---|---|---|---|---|---|---|---|---|---|
| Fatty- | pYES2 | Con- | | | 20:1 | −20:1 | 20:2 | 20:3 | 20:3 |
| acid | Blank | trol | 18:2 | 18:3 | Δ8 | Δ11 | Δ11.14 | Ω3 | Ω6 |
| 16:0 Δ | 16.9 | 20.4 | 27.7 | 24.4 | 16.2 | 21 | 17.6 | 19.5 | 22.8 |
| 16:1 Δ9 | 44.7 | 44.1 | 13.2 | 9.6 | 37.4 | 39.4 | 38.3 | 36.9 | 30.7 |
| 18:0 | 6.1 | 6.9 | 10.54 | 9.8 | 4.7 | 7.9 | 6.3 | 6.8 | 8.2 |
| 18:1 Δ9 | 31.72 | 28.1 | 8.77 | 6 | 15 | 26 | 29.5 | 25.6 | 21.1 |
| 18:2 Δ5, 9 | | 0.17 | 0 | 0 | 0 | 0.09 | 0.21 | 0.09 | 9 |
| 18:2 Δ9, 12 | | — | 39.7 | — | — | — | — | — | — |
| 18:3 Δ9, 12, 15 | | — | | 49.9 | — | — | — | — | — |
| 20:1 Δ8 | | — | | — | 25.5 | — | — | — | — |
| 20:1 Δ11 | | — | | — | — | 5.41 | — | — | — |
| 20:2 Δ5, 11 | | — | | — | — | 0.21 | — | — | — |
| 20:2 Δ11, 14 | | — | — | — | — | — | 6.48 | — | — |
| 20:3 Δ5, 11, 14 | | — | | | | | 0.76 | — | — |
| 20:3 Δ11, 14, 17 | | — | — | — | — | — | — | 9.83 | — |
| 20:3 Δ8, 11, 14 | | — | — | — | — | — | — | — | 13.69 |
| 20:4 Δ5, 11, 14, 17 | | — | — | — | — | — | — | 1.16 | — |
| 20:4 Δ5, 8, 11, 14 | | — | — | — | — | — | — | — | 3.08 |

The data represent mol % fatty acids of cis-fatty acids.

Further feeding experiments have revealed that C18:1Δ9 was not desaturated in the presence of C18:2Δ9,11 or C18:3Δ9,12,15 or C20:1Δ8 fatty acids, while C18:1 is also desaturated in the presence of C20:1Δ1, C20:2Δ11,14 and C20:3Δ8,11,14. Also, no desaturation took place in the presence of C20:3Δ8,11,14.

When using the protease-deficient yeast strain C13BYS86 (Kunze I. et al., Biochemica et Biophysica Acta (1999) 1410:287-298) for expressing the Phaeodactylum tricornutum Δ5-desaturase on complete medium with blasticidin, it was found that C20:4 Δ8,11,14,17 as substrate of Δ5-desaturase gave a conversion rate of 20% and was thus equally well converted as C20:3Δ8,11,14. As an alternative, the auxotrophism markers leu2, ura3 or his can also be used for gene expression.

In a further coexpression experiment of Phaeodactylum Δ5-desaturase and Physcomitrella Δ6-elongase, the strain used was UTL7A (Warnecke et al., J. Biol. Chem. (1999) 274(19):13048-13059), A5-desaturase converting approximately 10% of C20:3 A8,11,14 into C20:4Δ5,8,11,14.

Further feeding experiment with a wide range of other fatty acids alone or in combination (for example linoleic acid, 20:3 Δ5,11,14-fatty acid, α- or γ-linolenic acid, stearidonic acid, arachidonic acid, eicosapentaenoic acid and the like) can be carried out for confirming the substrate specificity and substrate selectivity of these desaturases in greater detail.

TABLE 8
Result of coexpressing a Phaeodactylum tricornutum
Δ5-acyl lipid desaturase and a moss Δ6-elongase in
yeast based on the expression vectors pYES2 and pYES6

| | |
|---|---|
| | pYES2-Elo and |
| pYES2-Elo | pYES6-Ptd5 |

| | +18:3 | +18:4 | +18:3 | +18:4 |
|---|---|---|---|---|
| 16:0 | 15.0 | 14.8 | 15.6 | 15.1 |
| 16:1Δ9 | 27.7 | 29.2 | 27.5 | 29.0 |
| 18:0 | 5.6 | 6.3 | 5.7 | 6.4 |
| 18:1Δ9 | 17.1 | 30.8 | 27.4 | 31.6 |
| 18:3Δ6, 9, 12 | 7.60 | — | 7.8 | — |
| 18:4Δ6, 9, 12, 15 | — | 6.71 | — | 6.4 |
| 20:3Δ8, 11, 14 | 15.92 | — | 13.55 | — |
| 20:4Δ5, 8, 11, 14 | — | — | 1.31 | — |
| 20:4Δ8, 11, 14, 17 | — | 11.4 | — | 10.31 |
| 20:5Δ5, 8, 11, 14, 17 | — | — | — | 0.53 |

The substrate conversions reveal that the Phaeodactylum Δ5-desaturase and the Physcomitrella patens A6 elongase which were used are suitable with regard to substrate activity and in particular substrate specificity for producing arachidonic acid or eicosapentaenoic acid with the aid of sequences according to the invention.

The fragmentation patterns and mass spectra of DMOX derivatives of standards and the peak fractions of the fatty acids shown in Tables 6, 7 and 8 and identified by GC show, in comparison, identical results, thus confirming the respective position of the double bond beyond simple GC detection.

### Example 18

### Purification of the Desired Product from Transformed Organisms

The recovery of the desired product from plant material or fungi, algae, ciliates, animal cells or the supernatant of the above-described cultures can be performed by various methods known in the art. If the desired product is not secreted from the cells, the cells can be harvested from the culture by low-speed centrifugation, the cells can be lysed by standard techniques, such as mechanical force or sonication. Organs of plants can be separated mechanically from other tissue or other organs. Following homogenization, the cell debris is removed by centrifugation, and the supernatant fraction comprising the soluble proteins is retained for further purification of the desired compounds. If the product is secreted from desired cells, then the cells are removed from the culture by low-speed centrifugation, and the supernatant fraction is retained for further purification.

The supernatant fraction from each purification method is subjected to chromatography with a suitable resin, the desired molecule either being retained on the chromatography resin while many of the impurities of the sample are not, or the impurities being retained by the resin while the sample is not. These chromatography steps can be repeated if necessary, using the same or different chromatography resins. The skilled worker is familiar with the selection of suitable chromatography resins and their most effective application for a particular molecule to be purified. The purified product can be concentrated by filtration or ultrafiltration, and stored at a temperature at which the stability of the product is maximized.

A wide spectrum of purification methods is known in the art, and the above purification method is not intended to be limiting. These purification methods are described, for example, in Bailey, J. E., & Ollis, D. F., Biochemical Engineering Fundamentals, McGraw-Hill: New York (1986).

The identity and purity of the isolated compounds may be assessed by techniques which are standard in the art. They include high-performance liquid chromatography (HPLC), spectroscopic methods, staining methods, thin-layer chromatography, in particular thin-layer chromatography and flame ionization detection (IATROSCAN, Iatron, Tokyo, Japan), NIRS, enzyme assays or microbiological tests. Such analytical methods are reviewed in: Patek et al. (1994) Appl. Environ. Microbiol. 60:133-140; Malakhova et al. (1996) Biotekhnologiya 11:27-32; and Schmidt et al. (1998) Bioprocess Engineer. 19:67-70. Ulmann's Encyclopedia of Industrial Chemistry (1996) Vol. A27, VCH Weinheim, pp. 89-90, pp. 521-540, pp. 540-547, pp. 559-566, pp. 575-581 and pp. 581-587; Michal, G (1999) Biochemical Pathways: An Atlas of Biochemistry and Molecular Biology, John Wiley and Sons; Fallon, A., et al. (1987) Applications of HPLC in Biochemistry in: Laboratory Techniques in Biochemistry and Molecular Biology, Vol. 17.

Equivalents

The skilled worker recognizes, or will be able to ascertain, a number of equivalents of the specific use forms according to the invention described herein by using no more than routine experiments. These equivalents are intended to be encompassed by the claims.

We claim:

1. An expression cassette with a structure selected from the group consisting of:
a) L1-promoter-structural gene-L2,
b) L1-promoter-structural gene-L2-L1-promoter-structural gene-L2,
c) L1-promoter-structural gene-L2-L1-promoter-structural gene-L2-L1-promoter-structural gene-L2,
where L1, L2, promoter and structural gene have the following meanings:
L1=SEQ ID NO: 32 or a sequence comprising equivalent restriction cleavage sites,
L2=independently of one another SEQ ID NO: 33, 34 or 35 or sequences comprising equivalent restriction cleavage sites,
promoter=plant promoter
structural gene=a nucleic acid sequence which can be expressed in plants.

2. An expression cassette as claimed in claim 1, wherein the structural gene is a biosynthesis gene.

3. An expression cassette as claimed in claim 1 or 2, wherein the structural gene is a biosynthesis gene of the lipid or fatty acid metabolism.

4. An expression cassette as claimed in any of claims 1 to 3, wherein the structural gene is a plant gene.

5. An expression cassette as claimed in any of claims 1 to 4, wherein the gene is a nucleic acid sequence encoding proteins selected from the group consisting of:
acyl-CoA dehydrogenase(s), acyl-ACP[=acyl carrier protein] desaturase(s), acyl-ACP thioesterase(s), fatty acid acyl transferase(s), fatty acid synthase(s), fatty acid hydroxylase(s), acetyl-coenzyme A carboxylase(s), acyl-coenzyme A oxidase(s), fatty acid desaturase(s), fatty acid acetylenases, lipoxygenases, triacylglycerol lipases, allenoxide synthases, hydroperoxide lyases or fatty acid elongase(s).

6. An expression cassette as claimed in any of claims 1 to 5, wherein the gene is a nucleic acid sequence selected from the group consisting of:
a) a nucleic acid sequence with the sequence shown in SEQ ID NO: 1, SEQ ID NO: 3, SEQ ID NO: 5 or SEQ ID NO: 11,
b) nucleic acid sequences which, owing to the degeneracy of the genetic code, are obtained by backtranslating the amino acid sequences shown in SEQ ID NO: 2, SEQ ID NO: 4, SEQ ID NO: 6 or SEQ ID NO: 12,
c) derivatives of the nucleic acid sequence shown in SEQ ID NO: 1, SEQ ID NO: 3, SEQ ID NO: 5 or SEQ ID NO: 11, which encode polypeptides with the amino acid sequences shown in SEQ ID NO: 2, SEQ ID NO: 4, SEQ ID NO: 6 or SEQ ID NO: 12 and have at least 50% homology at the amino acid level, without essentially reducing the enzymatic action of the polypeptides.

7. The use of expression cassettes as claimed in any of claims 1 to 8 in a method for the production of fatty acid esters with an increased content of polyunsaturated fatty acids having at least two double bonds, which comprises introducing the expression cassette into an organism producing fatty acid esters, growing the organism and isolating the fatty acid esters present in the organism.

8. A method as claimed in claim 7, wherein the fatty acid esters produced by the method comprise polyunsaturated C₁₈-, C₂₀- or C₂₂-fatty acid molecules with at least two double bonds in the fatty acid ester.

9. A method as claimed in claim 7 or 8, wherein the C₁₈-, C₂₀- or C₂₂-fatty acid molecules are isolated from the organism in the form of an oil or lipid.

10. A method as claimed in any of claims 7 to 9, wherein the organism is a transgenic plant.

11. A method as claimed in any of claims 7 to 10, wherein the fatty acid esters contain C₁₈-, C₂₀- or C₂₂-fatty acids with three, four or five double bonds in the fatty acid ester.

12. A method as claimed in any of claims 7 to 11, wherein the polyunsaturated fatty acids present in the fatty acid esters are liberated.

13. A vector comprising an expression cassette as claimed in any of claims 1 to 6.

14. An organism comprising at least one expression cassette as claimed in any of claims 1 to 6 or at least one vector as claimed in claim 13.

15. An organism as claimed in claim 14, which is a plant.

16. A transgenic plant comprising a functional or nonfunctional nucleic acid sequence in an expression cassette as claimed in any of claims 1 to 6.

17. The use of an expression cassette as claimed in any of claims 1 to 6 for generating transgenic plants.


SEQUENCE LISTING (35 sequences)

SEQ ID NO: 1 - DNA, 1652 bp, Phaeodactylum tricornutum
  CDS (115)..(1524)
gacccaacaaacccaacaatcccaacaatcccatcaacaggaattgggtttcgttgagtcaataattgctagaatccaaacagacagacagagaccaaccgcatctattacagaatggctccggatgcggataagcttcgacaacgccagacgactgcggtagcgaagcacaatgctgctaccatatcgacgcaggaacgcctttgcagtctgtcttcgctcaaaggcgaagaagtctgcatcgacggaatcatctatgacctccaatcattcgatcatcccgggggtgaaacgatcaaaatgtttggtggcaacgatgtcactgtacagtacaagatgattcacccgtaccataccgagaagcatttggaaaagatgaagcgtgtcggcaaggtgacggatttcgtctgcgagtacaagttcgataccgaatttgaacgcgaaatcaaacgagaagtcttcaagattgtgcgacgaggcaaggatttcggtactttgggatggttcttccgtgcgttttgctacattgccattttcttctacctgcagtaccattgggtcaccacgggaacctcttggctgctggccgtggcctacggaatctcccaagcgatgattggcatgaatgtccagcacgatgccaaccacggggccacctccaagcgtccctgggtcaacgacatgctaggcctcggtgcggattttattggtggttccaagtggctctggcaggaacaacactggacccaccacgcttacaccaatcacgccgagatggatcccgatagctttggtgccgaaccaatgctcctattcaacgactatcccttggatcatcccgctcgtacctggctacatcgctttcaagcattcttttacatgcccgtcttggctggatactggttgtccgctgtcttcaatccacaaattcttgacctccagcaacgcggcgcactttccgtcggtatccgtctcgacaacgctttcattcactcgcgacgcaagtatgcggttttctggcgggctgtgtacattgcggtgaacgtgattgctccgttttacacaaactccggcctcgaatggtcctggcgtgtctttggaaacatcatgctcatgggtgtggcggaatcgctcgcgctggcggtcctgttttcgttgtcgcacaatttcgaatccgcggatcgcgatccgaccgccccactgaaaaagacgggagaaccagtcgactggttcaagacacaggtcgaaacttcctgcacttacggtggattcctttccggttgcttcacgggaggtctcaactttcaggttgaacaccacttgttcccacgcatgagcagcgcttggtatccctacattgcccccaaggtccgcgaaatttgcgccaaacacggcgtccactacgcctactacccgtggatccaccaaaactttctctccaccgtccgctacatgcacgcggccgggaccggtgccaactggcgccagatggccagagaaaatcccttgaccggacgggcgtaaaagtacacgacacgaccaaaggtggcgtatggtgatctctagaaaacagacatagcctactggaaatatcgacgtccaaacaataattttaaagactatttttctgcgtaaaaaaaaaaaaaaaaaaa

SEQ ID NO: 2 - protein, 469 aa, Phaeodactylum tricornutum
MAPDADKLRQRQTTAVAKHNAATISTQERLCSLSSLKGEEVCIDGIIYDLQSFDHPGGETIKMFGGNDVTVQYKMIHPYHTEKHLEKMKRVGKVTDFVCEYKFDTEFEREIKREVFKIVRRGKDFGTLGWFFRAFCYIAIFFYLQYHWVTTGTSWLLAVAYGISQAMIGMNVQHDANHGATSKRPWVNDMLGLGADFIGGSKWLWQEQHWTHHAYTNHAEMDPDSFGAEPMLLFNDYPLDHPARTWLHRFQAFFYMPVLAGYWLSAVFNPQILDLQQRGALSVGIRLDNAFIHSRRKYAVFWRAVYIAVNVIAPFYTNSGLEWSWRVFGNIMLMGVAESLALAVLFSLSHNFESADRDPTAPLKKTGEPVDWFKTQVETSCTYGGFLSGCFTGGLNFQVEHHLFPRMSSAWYPYIAPKVREICAKHGVHYAYYPWIHQNFLSTVRYMHAAGTGANWRQMARENPLTGRA

SEQ ID NO: 3 - DNA, 1434 bp, Phaeodactylum tricornutum
  CDS (1)..(1434)
atgggcaaaggaggggacgctcgggcctcgaagggctcaacggcggctcgcaagatcagttggcaggaagtcaagacccacgcgtctccggaggacgcctggatcattcactccaataaggtctacgacgtgtccaactggcacgaacatcccggaggcgccgtcattttcacgcacgccggtgacgacatgacggacattttcgctgcctttcacgcacccggatcgcagtcgctcatgaagaagttctacattggcgaattgctcccggaaaccaccggcaaggagccgcagcaaatcgcctttgaaaagggctaccgcgatctgcgctccaaactcatcatgatgggcatgttcaagtccaacaagtggttctacgtctacaagtgcctcagcaacatggccatttgggccgccgcctgtgctctcgtcttttactcggaccgcttctgggtacacctggccagcgccgtcatgctgggaacattctttcagcagtcgggatggttggcacacgactttctgcaccaccaggtcttcaccaagcgcaagcacggggatctcggaggactcttttgggggaacctcatgcagggttactccgtacagtggtggaaaaacaagcacaacggacaccacgccgtccccaacctccactgctcctccgcagtcgcgcaagatggggacccggacatcgataccatgccccttctcgcctggtccgtccagcaagcccagtcttaccgggaactccaagccgacggaaaggattcgggtttggtcaagttcatgatccgtaaccaatcctacttttactttcccatcttgttgctcgcccgcctgtcgtggttgaacgagtccttcaagtgcgcctttgggcttggagctgcgtcggagaacgctgctctcgaactcaaggccaagggtcttcagtacccccttttggaaaaggctggcatcctgctgcactacgcttggatgcttacagtttcgtccggctttggacgcttctcgttcgcgtacaccgcattttactttctaaccgcgaccgcgtcctgtggattcttgctcgccattgtctttggcctcggccacaacggcatggccacctacaatgccgacgcccgtccggacttctggaagctccaagtcaccacgactcgcaacgtcacgggcggacacggtttcccccaagcctttgtcgactggttctgtggtggcctccagtaccaagtcgaccaccacttattccccagcctgccccgacacaatctggccaagacacacgcactggtcgaatcgttctgcaaggagtggggtgtccagtaccacgaagccgaccttgtggacgggaccatggaagtcttgcaccatttgggcagcgtggccggcgaattcgtcgtggattttgtacgcgatggacccgccatgtaa

SEQ ID NO: 4 - protein, 477 aa, Phaeodactylum tricornutum
MGKGGDARASKGSTAARKISWQEVKTHASPEDAWIIHSNKVYDVSNWHEHPGGAVIFTHAGDDMTDIFAAFHAPGSQSLMKKFYIGELLPETTGKEPQQIAFEKGYRDLRSKLIMMGMFKSNKWFYVYKCLSNMAIWAAACALVFYSDRFWVHLASAVMLGTFFQQSGWLAHDFLHHQVFTKRKHGDLGGLFWGNLMQGYSVQWWKNKHNGHHAVPNLHCSSAVAQDGDPDIDTMPLLAWSVQQAQSYRELQADGKDSGLVKFMIRNQSYFYFPILLLARLSWLNESFKCAFGLGAASENAALELKAKGLQYPLLEKAGILLHYAWMLTVSSGFGRFSFAYTAFYFLTATASCGFLLAIVFGLGHNGMATYNADARPDFWKLQVTTTRNVTGGHGFPQAFVDWFCGGLQYQVDHHLFPSLPRHNLAKTHALVESFCKEWGVQYHEADLVDGTMEVLHHLGSVAGEFVVDFVRDGPAM

SEQ ID NO: 5 - DNA, 1651 bp, Phaeodactylum tricornutum
  CDS (67)..(1554)
gaagaaggaacatataaaagtaagccatctcctcggcaccatctaaagacctaatatctactcgtcatggttcgcttttcaacagccgctctactttctctgtcgacattgacaacttcatgtattggtgccttccagctgtcttcgccagcacaacttccgacaagtaggcttcgtcggcatacgaacacggcgccgctttcggccgtggccgtcgactccggttcttccgatccggccttggtaggcaacctcccccttcccaacaacaatgataatgaggacaagaaccgtagaatgccaatgatggacttgaaaggtattgctctgtctggtctcaaagggcaagctctttccgtccgagcggaagattttcctcaggcgaaagacttgcgtgccgtcattccgaaagattgcttcgaacccgacacggccaaatcgttgggatatctttccgtttcaactatggggacaattctctgctccgtcgtcggcgcgaacctccttagtgtgctcgatccctccaatccattaacctggcctctctgggcggcctacggtgccgtcacggggacggtcgccatggggctttgggtgctggcccacgaatgcggacacggcgccttttccaaaaaccgatccctccaggatgccgtggggtacattatccattccatcatgctggtgccatactttagttggcagcgatcgcatgccgtgcatcaccagtataccaatcatatggaactgggggaaacacacgttcctgatcgagccgataaggagggcgagaagagcctggcgctccgccagttcatgttggattcctttggtaaagacaagggcatgaaagcatacggaggcctccagtcgtttttgcatctcatcgtgggatggccagcctacctcctgatcggtgcgaccggtggacccgaccgtggtatgaccaaccatttttatcccaaccctttgtcgacgccaacacagcccaagaaagaacttttccctgggaactggaaagaaaaggtctaccagtcagatattggaatcgccgccgttgtcggcgccctcattgcttggaccgccacttcgggtctagcccccgtcatggccttgtacggtggtcccttgatcgtcattaatgcctggctggtactgtacacgtggttgcaacatacagataccgatgttccgcacttttcctccgacaaccacaactttgtcaagggcgcactgcatacgatcgatcgtccctacgacaaacttgatccctggggaatcatagactttctgcaccacaagattggaacaacgcatgtggcacaccattttgacagtactatcccccactataaggctcagattgctaccgatgccatcaaagccaagtttccagaagtgtacctctatgacccgacaccaattccacaagccatgtggcgcgtcgccaagggatgtactgcagtagagcaacgcggtgacgcctgggtgtggaaaaacgaaggaatagaagatttggtggaacatcgtcaaagcaaattatcgagcgaataaagcaacatatcgctttatggaagaacaaacgtccattgtgtaaaaccctgataatttcaatattgtgttttgttttaaaaaaaaaaaaaaaaaaaaa

SEQ ID NO: 6 - protein, 495 aa, Phaeodactylum tricornutum
MVRFSTAALLSLSTLTTSCIGAFQLSSPAQLPTSRLRRHTNTAPLSAVAVDSGSSDPALVGNLPLPNNNDNEDKNRRMPMMDLKGIALSGLKGQALSVRAEDFPQAKDLRAVIPKDCFEPDTAKSLGYLSVSTMGTILCSVVGANLLSVLDPSNPLTWPLWAAYGAVTGTVAMGLWVLAHECGHGAFSKNRSLQDAVGYIIHSIMLVPYFSWQRSHAVHHQYTNHMELGETHVPDRADKEGEKSLALRQFMLDSFGKDKGMKAYGGLQSFLHLIVGWPAYLLIGATGGPDRGMTNHFYPNPLSTPTQPKKELFPGNWKEKVYQSDIGIAAVVGALIAWTATSGLAPVMALYGGPLIVINAWLVLYTWLQHTDTDVPHFSSDNHNFVKGALHTIDRPYDKLDPWGIIDFLHHKIGTTHVAHHFDSTIPHYKAQIATDAIKAKFPEVYLYDPTPIPQAMWRVAKGCTAVEQRGDAWVWKNEGIEDLVEHRQSKLSSE

SEQ ID NO: 7 - DNA, 1578 bp, Physcomitrella patens
  CDS (1)..(1578)
atggtattcgcgggcggtggacttcagcagggctctctcgaagaaaacatcgacgtcgagcacattgccagtatgtctctcttcagcgacttcttcagttatgtgtcttcaactgttggttcgtggagcgtacacagtatacaacctttgaagcgcctgacgagtaagaagcgtgtttcggaaagcgctgccgtgcaatgtatatcagctgaagttcagagaaattcgagtacccagggaactgcggaggcactcgcagaatcagtcgtgaagcccacgagacgaaggtcatctcagtggaagaagtcgacacaccccctatcagaagtagcagtacacaacaagccaagcgattgctggattgttgtaaaaaacaaggtgtatgatgtttccaattttgcggacgagcatcccggaggatcagttattagtacttattttggacgagacggcacagatgttttctctagttttcatgcagcttctacatggaaaattcttcaagacttttacattggtgacgtggagagggtggagccgactccagagctgctgaaagatttccgagaaatgagagctcttttcctgagggagcaacttttcaaaagttcgaaattgtactatgttatgaagctgctcacgaatgttgctatttttgctgcgagcattgcaataatatgttggagcaagactatttcagcggttttggcttcagcttgtatgatggctctgtgtttccaacagtgcggatggctatcccatgattttctccacaatcaggtgtttgagacacgctggcttaatgaagttgtcgggtatgtgatcggcaacgccgttctggggtttagtacagggtggtggaaggagaagcataaccttcatcatgctgctccaaatgaatgcgatcagacttaccaaccaattgatgaagatattgatactctccccctcattgcctggagcaaggacatactggccacagttgagaataagacattcttgcgaatcctccaataccagcatctgttcttcatgggtctgttatttttcgcccgtggtagttggctcttttggagctggagatatacctctacagcagtgctctcacctgtcgacaggttgttggagaagggaactgttctgtttcactacttttggttcgtcgggacagcgtgctatcttctccctggttggaagccattagtatggatggcggtgactgagctcatgtccggcatgctgctgggctttgtatttgtacttagccacaatgggatggaggtttataattcgtctaaagaattcgtgagtgcacagatcgtatccacacgggatatcaaaggaaacatattcaacgactggttcactggtggccttaacaggcaaatagagcatcatcttttcccaacaatgcccaggcataatttaaacaaaatagcacctagagtggaggtgttctgtaagaaacacggtctggtgtacgaagacgtatctattgctaccggcacttgcaaggttttgaaagcattgaaggaagtcgcggaggctgcggcagagcagcatgctaccaccagttaa

SEQ ID NO: 8 - protein, 525 aa, Physcomitrella patens
MVFAGGGLQQGSLEENIDVEHIASMSLFSDFFSYVSSTVGSWSVHSIQPLKRLTSKKRVSESAAVQCISAEVQRNSSTQGTAEALAESVVKPTRRRSSQWKKSTHPLSEVAVHNKPSDCWIVVKNKVYDVSNFADEHPGGSVISTYFGRDGTDVFSSFHAASTWKILQDFYIGDVERVEPTPELLKDFREMRALFLREQLFKSSKLYYVMKLLTNVAIFAASIAIICWSKTISAVLASACMMALCFQQCGWLSHDFLHNQVFETRWLNEVVGYVIGNAVLGFSTGWWKEKHNLHHAAPNECDQTYQPIDEDIDTLPLIAWSKDILATVENKTFLRILQYQHLFFMGLLFFARGSWLFWSWRYTSTAVLSPVDRLLEKGTVLFHYFWFVGTACYLLPGWKPLVWMAVTELMSGMLLGFVFVLSHNGMEVYNSSKEFVSAQIVSTRDIKGNIFNDWFTGGLNRQIEHHLFPTMPRHNLNKIAPRVEVFCKKHGLVYEDVSIATGTCKVLKALKEVAEAAAEQHATTS

SEQ ID NO: 9 - DNA, 873 bp, Physcomitrella patens
  CDS (1)..(873)
atggaggtcgtggagagattctacggtgagttggatgggaaggtctcgcagggcgtgaatgcattgctgggtagttttggggtggagttgacggatacgcccactaccaaaggcttgcccctcgttgacagtcccacacccatcgtcctcggtgtttctgtatacttgactattgtcattggagggcttttgtggataaaggccagggatctgaaaccgcgcgcctcggagccatttttgctccaagctttggtgcttgtgcacaacctgttctgttttgcgctcagtctgtatatgtgcgtgggcatcgcttatcaggctattacctggcggtactctctctggggcaatgcatacaatcctaaacataaagagatggcgattctggtatacttgttctacatgtctaagtacgtggaattcatggataccgttatcatgatactgaagcgcagcaccaggcaaataagcttcctccacgtttatcatcattcttcaatttccctcatttggtgggctattgctcatcacgctcctggcggtgaagcatattggtctgcggctctgaactcaggagtgcatgttctcatgtatgcgtattacttcttggctgcctgccttcgaagtagcccaaagttaaaaaataagtaccttttttggggcaggtacttgacacaattccaaatgttccagtttatgctgaacttagtgcaggcttactacgacatgaaaacgaatgcgccatatccacaatggctgatcaagattttgttctactacatgatctcgttgctgtttcttttcggcaatttttacgtacaaaaatacatcaaaccctctgacggaaagcaaaagggagctaaaactgagtga

SEQ ID NO: 10 - protein, 290 aa, Physcomitrella patens
MEVVERFYGELDGKVSQGVNALLGSFGVELTDTPTTKGLPLVDSPTPIVLGVSVYLTIVIGGLLWIKARDLKPRASEPFLLQALVLVHNLFCFALSLYMCVGIAYQAITWRYSLWGNAYNPKHKEMAILVYLFYMSKYVEFMDTVIMILKRSTRQISFLHVYHHSSISLIWWAIAHHAPGGEAYWSAALNSGVHVLMYAYYFLAACLRSSPKLKNKYLFWGRYLTQFQMFQFMLNLVQAYYDMKTNAPYPQWLIKILFYYMISLLFLFGNFYVQKYIKPSDGKQKGAKTE

SEQ ID NO: 11 - DNA, 1526 bp, Phaeodactylum tricornutum
  CDS (92)..(1402)
gcttccgttagcgtcccatagtttgttacacttggctgtgaaacgaatacgttcttggtctacttactacaacgaagcaaccaccagcagcatgggtaagggaggtcaacgagctgtagctcccaagagtgccaccagctctactggcagtgctacccttagccaaagcaaggaacaggtatggacttcgtcgtacaaccctctggcgaaggattccccggagctgccaaccaaaggccaaatcaaggccgtcattccgaaggaatgtttccaacgctcagccttttggtctaccttctacctgatgcgcgatctcgccatggctgccgccttttgctacggaacctcacaggtcctctccaccgaccttccccaagacgccacgctcattctgccctgggctctcggctggggcgtctacgccttttggatgggaaccattctcaccgggccttgggtagttgcgcacgaatgtggacacggcgcttactccgactcccagacgttcaatgacgtggtcggctttatcgtccaccaagctttgctcgtcccctactttgcctggcagtacacccacgcgaaacaccaccgtcgaaccaaccatctggtggacggcgagtcccacgtcccttctaccgccaaggataacggcctcgggccgcacaacgagcgaaactccttctacgccgcgtggcacgaggccatgggagacggcgcctttgccgtctttcaagtctggtcgcacttgttcgtcggctggcctctctacttggccggtctggccagtaccggaaagcttgcgcacgaaggttggtggctggaagaacggaacgcgattgcggatcactttcgacccagctctcccatgttccccgccaagatccgtgccaagattgccctttccagcgcgacggaactcgccgtgctcgctggactcttgtatgtcggtacacaggtcggacaccttcccgtcctgctgtggtactggggaccgtacacctttgtcaacgcttggcttgtactctacacgtggctgcagcatacggacccgtccatcccgcactacggtgaaggcgagtggacctgggtcaagggcgcgctctctaccattgatcgagactacggcatcttcgatttctttcaccacaccatcggttccacgcacgtggtacaccatttgttccacgaaatgccctggtacaatgccggcattgccacgcaaaaggtcaaggaatttttggaaccccagggcttgtacaattacgatccgaccccctggtacaaggccatgtggcgcattgcccggacctgtcactatgtggagtcaaacgagggtgtgcagtatttcaagagtatggaaaacgtgccgctgactaaggatgtgcgaaacaaagccgcatgagaaaaagtgccaccgacgcataattttacaatcctaccaacaagaccaacattatatggttttcgcttaaaagatagttttttctaccatctgtgtagtcggcacaaaaaaaaaaaaaaaaaaa

SEQ ID NO: 12 - protein, 436 aa, Phaeodactylum tricornutum
MGKGGQRAVAPKSATSSTGSATLSQSKEQVWTSSYNPLAKDSPELPTKGQIKAVIPKECFQRSAFWSTFYLMRDLAMAAAFCYGTSQVLSTDLPQDATLILPWALGWGVYAFWMGTILTGPWVVAHECGHGAYSDSQTFNDVVGFIVHQALLVPYFAWQYTHAKHHRRTNHLVDGESHVPSTAKDNGLGPHNERNSFYAAWHEAMGDGAFAVFQVWSHLFVGWPLYLAGLASTGKLAHEGWWLEERNAIADHFRPSSPMFPAKIRAKIALSSATELAVLAGLLYVGTQVGHLPVLLWYWGPYTFVNAWLVLYTWLQHTDPSIPHYGEGEWTWVKGALSTIDRDYGIFDFFHHTIGSTHVVHHLFHEMPWYNAGIATQKVKEFLEPQGLYNYDPTPWYKAMWRIARTCHYVESNEGVQYFKSMENVPLTKDVRNKAA

SEQ ID NO: 13 - DNA, 3598 bp, Unknown
  Sequenz stellt eine pflanzliche Promotor-Terminator-Expressionskassette in Vektor pUC19 dar
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcggcgcgccgagctcctcgagcaaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgctgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcatagccagcccaccgcggtgggcggccgcctgcagtctagaaggcctcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccgtcgacgaattcgagctcggcgcgccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 14 - DNA, 3590 bp, Unknown
  Sequenz stellt eine pflanzliche Promotor-Terminator-Expressionskassette in Vektor pUC19 dar
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcggcgcgccgagctcctcgagcaaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgctgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcatagccagcggatccgatatcgggcccgctagcgttaaccctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccgtcgacgaattcgagctcggcgcgccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 15 - DNA, 3584 bp, Unknown
  Sequenz stellt eine pflanzliche Promotor-Terminator-Expressionskassette in Vektor pUC19 dar
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcggcgcgccgagctcctcgagcaaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgctgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcatagccagcagatctgccggcatcgatcccgggccatggcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccgtcgacgagctcggcgcgccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 16 - DNA, 4507 bp, Unknown
  Sequenz stellt eine pflanzliche Promotor-Terminator-Expressionskassette in Vektor pUC19 dar
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcggcgcgccgagctcctcgagcaaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgctgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcatagccagcccaccgcggtgggcggccgcctgcagtctagaaggcctcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccgtcgagcaaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgctgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcatagccagcggatccgatatcgggcccgctagcgttaaccctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccgtcgacgaattcgagctcggcgcgccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 17 - DNA, 5410 bp, Unknown
  Sequenz stellt eine pflanzliche Promotor-Terminator-Expressionskassette in Vektor pUC19 dar
ttttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcatagccagcggatccgatatcgggcccgctagcgttaaccctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccgtcgagcaaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgctgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcatagccagcagatctgccggcatcgatcccgggccatggcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccgtcgacgagctcggcgcgccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcggcgcgccgagctcctcgagcaaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgctgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcatagccagcccaccgcggtgggcggccgcctgcagtctagaaggcctcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccgtcgagcaaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgctgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaata

SEQ ID NO: 18 - DNA, 648 bp, Phaeodactylum tricornutum
  CDS (1)..(648)
  .
tggtggaaaaacaagcacaacggacaccacgccgtccccaacctccactgctcctccgcagtcgcgcaagatggggacccggacatcgataccatgccccttctcgcctggtccgtccagcaagcccagtcttaccgggaactccaagccgacggaaaggattcgggtttggtcaagttcatgatccgtaaccaatcctacttttactttcccatcttgttgctcgcccgcctgtcgtggttgaacgagtccttcaagtgcgcctttgggcttggagctgcgtcggagaacgctgctctcgaactcaaggccaagggtcttcagtacccccttttggaaaaggctggcatcctgctgcactacgcttggatgcttacagtttcgtccggctttggacgcttctcgttcgcgtacaccgcattttactttctaaccgcgaccgcgtcctgtggattcttgctcgccattgtctttggcctcggccacaacggcatggccacctacaatgccgacgcccgtccggacttctggaagctccaagtcaccacgactcgcaacgtcacgggcggacacggtttcccccaagcctttgtcgactggttctgtggtggcctccagtaccaagtcgaccaccacttattccccagc

SEQ ID NO: 19 - protein, 216 aa, Phaeodactylum tricornutum
WWKNKHNGHHAVPNLHCSSAVAQDGDPDIDTMPLLAWSVQQAQSYRELQADGKDSGLVKFMIRNQSYFYFPILLLARLSWLNESFKCAFGLGAASENAALELKAKGLQYPLLEKAGILLHYAWMLTVSSGFGRFSFAYTAFYFLTATASCGFLLAIVFGLGHNGMATYNADARPDFWKLQVTTTRNVTGGHGFPQAFVDWFCGGLQYQVDHHLFPS

SEQ ID NO: 20 - DNA, 12093 bp, Unknown
  pflanzlicher Expressionsvektor mit einer Promotor-Terminator-Expressionskassette
gatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattaattcccatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatgtgtaatacataaattgatgatatagctagcttagctcatcgggggatccgtcgaagctagcttgggtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctcccatgggccctcgactagagtcgagatctggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagcttgcatgcctgcaggtcgacggcgcgccgagctcctcgagcaaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgctgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcatagccagcccaccgcggtgggcggccgcctgcagtctagaaggcctcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccgtcgacgaattcgagctcggcgcgcctctagaggatcgatgaattcagatcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccttcgtccatttgtatgtgcatgccaaccacagggttcccca

SEQ ID NO: 21 - DNA, 12085 bp, Unknown
  pflanzlicher Expressionsvektor mit einer Promotor-Terminator-Expressionskassette
gatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattaattcccatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatgtgtaatacataaattgatgatatagctagcttagctcatcgggggatccgtcgaagctagcttgggtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctcccatgggccctcgactagagtcgagatctggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagcttgcatgcctgcaggtcgacggcgcgccgagctcctcgagcaaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgctgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcatagccagcggatccgatatcgggcccgctagcgttaaccctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccgtcgacgaattcgagctcggcgcgcctctagaggatcgatgaattcagatcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccttcgtccatttgtatgtgcatgccaaccacagggttcccca

SEQ ID NO: 22 - DNA, 12079 bp, Unknown
  pflanzlicher Expressionsvektor mit einer Promotor-Terminator-Expressionskassette
gatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattaattcccatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatgtgtaatacataaattgatgatatagctagcttagctcatcgggggatccgtcgaagctagcttgggtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctcccatgggccctcgactagagtcgagatctggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagcttgcatgcctgcaggtcgacggcgcgccgagctcctcgagcaaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgctgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcatagccagcagatctgccggcatcgatcccgggccatggcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccgtcgacgagctcggcgcgcctctagaggatcgatgaattcagatcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccttcgtccatttgtatgtgcatgccaaccacagggttcccca

SEQ ID NO: 23 - DNA, 13002 bp, Unknown
  pflanzlicher Expressionsvektor mit zwei Promotor-Terminator-Expressionskassetten
gatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattaattcccatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatgtgtaatacataaattgatgatatagctagcttagctcatcgggggatccgtcgaagctagcttgggtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctcccatgggccctcgactagagtcgagatctggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagcttgcatgcctgcaggtcgacggcgcgccgagctcctcgagcaaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgctgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcatagccagcccaccgcggtgggcggccgcctgcagtctagaaggcctcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccgtcgagcaaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgctgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcatagccagcggatccgatatcgggcccgctagcgttaaccctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccgtcgacgaattcgagctcggcgcgcctctagaggatcgatgaattcagatcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccttcgtccatttgtatgtgcatgccaaccacagggttcccca

SEQ ID NO: 24 - DNA, 13905 bp, Unknown
  pflanzlicher Expressionsvektor mit drei Promotor-Terminator-Expressionskassetten
gatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattaattcccatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatgtgtaatacataaattgatgatatagctagcttagctcatcgggggatccgtcgaagctagcttgggtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctcccatgggccctcgactagagtcgagatctggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagcttgcatgcctgcaggtcgacggcgcgccgagctcctcgagcaaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgctgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcatagccagcccaccgcggtgggcggccgcctgcagtctagaaggcctcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccgtcgagcaaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgctgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcatagccagcggatccgatatcgggcccgctagcgttaaccctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccgtcgagcaaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgctgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcatagccagcagatctgccggcatcgatcccgggccatggcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccgtcgacgagctcggcgcgcctctagaggatcgatgaattcagatcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccttcgtccatttgtatgtgcatgccaaccacagggttcccca

SEQ ID NO: 25 - DNA, 15430 bp, Unknown
  pflanz. Expressionsvektor mit zwei Promotor- Terminator-Expressionskassetten inseriert ist Physcomitrella patens Elongase und Desaturase
gatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattaattcccatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatgtgtaatacataaattgatgatatagctagcttagctcatcgggggatccgtcgaagctagcttgggtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctcccatgggccctcgactagagtcgagatctggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagcttgcatgcctgcaggtcgacggcgcgccgagctcctcgagcaaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgctgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcatagccagcccaccgcggtggaaaatggaggtcgtggagagattctacggtgagttggatgggaaggtctcgcagggcgtgaatgcattgctgggtagttttggggtggagttgacggatacgcccactaccaaaggcttgcccctcgttgacagtcccacacccatcgtcctcggtgtttctgtatacttgactattgtcattggagggcttttgtggataaaggccagggatctgaaaccgcgcgcctcggagccatttttgctccaagctttggtgcttgtgcacaacctgttctgttttgcgctcagtctgtatatgtgcgtgggcatcgcttatcaggctattacctggcggtactctctctggggcaatgcatacaatcctaaacataaagagatggcgattctggtatacttgttctacatgtctaagtacgtggaattcatggataccgttatcatgatactgaagcgcagcaccaggcaaataagcttcctccacgtttatcatcattcttcaatttccctcatttggtgggctattgctcatcacgctcctggcggtgaagcatattggtctgcggctctgaactcaggagtgcatgttctcatgtatgcgtattacttcttggctgcctgccttcgaagtagcccaaagttaaaaaataagtaccttttttggggcaggtacttgacacaattccaaatgttccagtttatgctgaacttagtgcaggcttactacgacatgaaaacgaatgcgccatatccacaatggctgatcaagattttgttctactacatgatctcgttgctgtttcttttcggcaatttttacgtacaaaaatacatcaaaccctctgacggaaagcaaaagggagctaaaactgagtgatctagaaggcctcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccgtcgagcaaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgctgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcatagccagcggatccatggtattcgcgggcggtggacttcagcagggctctctcgaagaaaacatcgacgtcgagcacattgccagtatgtctctcttcagcgacttcttcagttatgtgtcttcaactgttggttcgtggagcgtacacagtatacaacctttgaagcgcctgacgagtaagaagcgtgtttcggaaagcgctgccgtgcaatgtatatcagctgaagttcagagaaattcgagtacccagggaactgcggaggcactcgcagaatcagtcgtgaagcccacgagacgaaggtcatctcagtggaagaagtcgacacaccccctatcagaagtagcagtacacaacaagccaagcgattgctggattgttgtaaaaaacaaggtgtatgatgtttccaattttgcggacgagcatcccggaggatcagttattagtacttattttggacgagacggcacagatgttttctctagttttcatgcagcttctacatggaaaattcttcaagacttttacattggtgacgtggagagggtggagccgactccagagctgctgaaagatttccgagaaatgagagctcttttcctgagggagcaacttttcaaaagttcgaaattgtactatgttatgaagctgctcacgaatgttgctatttttgctgcgagcattgcaataatatgttggagcaagactatttcagcggttttggcttcagcttgtatgatggctctgtgtttccaacagtgcggatggctatcccatgattttctccacaatcaggtgtttgagacacgctggcttaatgaagttgtcgggtatgtgatcggcaacgccgttctggggtttagtacagggtggtggaaggagaagcataaccttcatcatgctgctccaaatgaatgcgatcagacttaccaaccaattgatgaagatattgatactctccccctcattgcctggagcaaggacatactggccacagttgagaataagacattcttgcgaatcctccaataccagcatctgttcttcatgggtctgttatttttcgcccgtggtagttggctcttttggagctggagatatacctctacagcagtgctctcacctgtcgacaggttgttggagaagggaactgttctgtttcactacttttggttcgtcgggacagcgtgctatcttctccctggttggaagccattagtatggatggcggtgactgagctcatgtccggcatgctgctgggctttgtatttgtacttagccacaatgggatggaggtttataattcgtctaaagaattcgtgagtgcacagatcgtatccacacgggatatcaaaggaaacatattcaacgactggttcactggtggccttaacaggcaaatagagcatcatcttttcccaacaatgcccaggcataatttaaacaaaatagcacctagagtggaggtgttctgtaagaaacacggtctggtgtacgaagacgtatctattgctaccggcacttgcaaggttttgaaagcattgaaggaagtcgcggaggctgcggcagagcagcatgctaccaccagttaagctagcgttaaccctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccgtcgacgaattcgagctcggcgcgcctctagaggatcgatgaattcagatcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccttcgtccatttgtatgtgcatgccaaccacagggttcccca

SEQ ID NO: 26 - protein, 290 aa, Unknown; identical to SEQ ID NO: 10
  pflanz. Expressionsvektor mit zwei Promotor- Terminator-Expressionskassetten inseriert ist Physcomitrella patens Elongase und Desaturase

SEQ ID NO: 27 - protein, 525 aa, Unknown; identical to SEQ ID NO: 8
  pflanz. Expressionsvektor mit zwei Promotor- Terminator-Expressionskassetten inseriert ist Physcomitrella patens Elongase und Desaturase

SEQ ID NO: 28 - DNA, 17752 bp, Unknown
  pflanz. Expressionsvektor mit 3 Promotor-Terminator- Expressionskassetten inseriert mit Physcomitrella Elongase + Desaturase + Phaeodactylum Desaturase
gatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattaattcccatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatgtgtaatacataaattgatgatatagctagcttagctcatcgggggatccgtcgaagctagcttgggtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctcccatgggccctcgactagagtcgagatctggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagcttgcatgcctgcaggtcgacggcgcgccgagctcctcgagcaaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgctgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcatagccagcccaccgcggtggaaaatggaggtcgtggagagattctacggtgagttggatgggaaggtctcgcagggcgtgaatgcattgctgggtagttttggggtggagttgacggatacgcccactaccaaaggcttgcccctcgttgacagtcccacacccatcgtcctcggtgtttctgtatacttgactattgtcattggagggcttttgtggataaaggccagggatctgaaaccgcgcgcctcggagccatttttgctccaagctttggtgcttgtgcacaacctgttctgttttgcgctcagtctgtatatgtgcgtgggcatcgcttatcaggctattacctggcggtactctctctggggcaatgcatacaatcctaaacataaagagatggcgattctggtatacttgttctacatgtctaagtacgtggaattcatggataccgttatcatgatactgaagcgcagcaccaggcaaataagcttcctccacgtttatcatcattcttcaatttccctcatttggtgggctattgctcatcacgctcctggcggtgaagcatattggtctgcggctctgaactcaggagtgcatgttctcatgtatgcgtattacttcttggctgcctgccttcgaagtagcccaaagttaaaaaataagtaccttttttggggcaggtacttgacacaattccaaatgttccagtttatgctgaacttagtgcaggcttactacgacatgaaaacgaatgcgccatatccacaatggctgatcaagattttgttctactacatgatctcgttgctgtttcttttcggcaatttttacgtacaaaaatacatcaaaccctctgacggaaagcaaaagggagctaaaactgagtgatctagaaggcctcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccgtcgagcaaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgctgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcatagccagcggatccatggtattcgcgggcggtggacttcagcagggctctctcgaagaaaacatcgacgtcgagcacattgccagtatgtctctcttcagcgacttcttcagttatgtgtcttcaactgttggttcgtggagcgtacacagtatacaacctttgaagcgcctgacgagtaagaagcgtgtttcggaaagcgctgccgtgcaatgtatatcagctgaagttcagagaaattcgagtacccagggaactgcggaggcactcgcagaatcagtcgtgaagcccacgagacgaaggtcatctcagtggaagaagtcgacacaccccctatcagaagtagcagtacacaacaagccaagcgattgctggattgttgtaaaaaacaaggtgtatgatgtttccaattttgcggacgagcatcccggaggatcagttattagtacttattttggacgagacggcacagatgttttctctagttttcatgcagcttctacatggaaaattcttcaagacttttacattggtgacgtggagagggtggagccgactccagagctgctgaaagatttccgagaaatgagagctcttttcctgagggagcaacttttcaaaagttcgaaattgtactatgttatgaagctgctcacgaatgttgctatttttgctgcgagcattgcaataatatgttggagcaagactatttcagcggttttggcttcagcttgtatgatggctctgtgtttccaacagtgcggatggctatcccatgattttctccacaatcaggtgtttgagacacgctggcttaatgaagttgtcgggtatgtgatcggcaacgccgttctggggtttagtacagggtggtggaaggagaagcataaccttcatcatgctgctccaaatgaatgcgatcagacttaccaaccaattgatgaagatattgatactctccccctcattgcctggagcaaggacatactggccacagttgagaataagacattcttgcgaatcctccaataccagcatctgttcttcatgggtctgttatttttcgcccgtggtagttggctcttttggagctggagatatacctctacagcagtgctctcacctgtcgacaggttgttggagaagggaactgttctgtttcactacttttggttcgtcgggacagcgtgctatcttctccctggttggaagccattagtatggatggcggtgactgagctcatgtccggcatgctgctgggctttgtatttgtacttagccacaatgggatggaggtttataattcgtctaaagaattcgtgagtgcacagatcgtatccacacgggatatcaaaggaaacatattcaacgactggttcactggtggccttaacaggcaaatagagcatcatcttttcccaacaatgcccaggcataatttaaacaaaatagcacctagagtggaggtgttctgtaagaaacacggtctggtgtacgaagacgtatctattgctaccggcacttgcaaggttttgaaagcattgaaggaagtcgcggaggctgcggcagagcagcatgctaccaccagttaagctagcgttaaccctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccgtcgagcaaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgctgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcatagccagcagatctaaaatggctccggatgcggataagcttcgacaacgccagacgactgcggtagcgaagcacaatgctgctaccatatcgacgcaggaacgcctttgcagtctgtcttcgctcaaaggcgaagaagtctgcatcgacggaatcatctatgacctccaatcattcgatcatcccgggggtgaaacgatcaaaatgtttggtggcaacgatgtcactgtacagtacaagatgattcacccgtaccataccgagaagcatttggaaaagatgaagcgtgtcggcaaggtgacggatttcgtctgcgagtacaagttcgataccgaatttgaacgcgaaatcaaacgagaagtcttcaagattgtgcgacgaggcaaggatttcggtactttgggatggttcttccgtgcgttttgctacattgccattttcttctacctgcagtaccattgggtcaccacgggaacctcttggctgctggccgtggcctacggaatctcccaagcgatgattggcatgaatgtccagcacgatgccaaccacggggccacctccaagcgtccctgggtcaacgacatgctaggcctcggtgcggattttattggtggttccaagtggctctggcaggaacaacactggacccaccacgcttacaccaatcacgccgagatggatcccgatagctttggtgccgaaccaatgctcctattcaacgactatcccttggatcatcccgctcgtacctggctacatcgctttcaagcattcttttacatgcccgtcttggctggatactggttgtccgctgtcttcaatccacaaattcttgacctccagcaacgcggcgcactttccgtcggtatccgtctcgacaacgctttcattcactcgcgacgcaagtatgcggttttctggcgggctgtgtacattgcggtgaacgtgattgctccgttttacacaaactccggcctcgaatggtcctggcgtgtctttggaaacatcatgctcatgggtgtggcggaatcgctcgcgctggcggtcctgttttcgttgtcgcacaatttcgaatccgcggatcgcgatccgaccgccccactgaaaaagacgggagaaccagtcgactggttcaagacacaggtcgaaacttcctgcacttacggtggattcctttccggttgcttcacgggaggtctcaactttcaggttgaacaccacttgttcccacgcatgagcagcgcttggtatccctacattgcccccaaggtccgcgaaatttgcgccaaacacggcgtccactacgcctactacccgtggatccaccaaaactttctctccaccgtccgctacatgcacgcggccgggaccggtgccaactggcgccagatggccagagaaaatcccttgaccggacgggcgtaaagatctgccggcatcgatcccgggccatggcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccgtcgacgagctcggcgcgcctctagaggatcgatgaattcagatcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccttcgtccatttgtatgtgcatgccaaccacagggttcccca

SEQ ID NO: 29 - protein, 290 aa, Unknown; identical to SEQ ID NO: 10
  pflanz. Expressionsvektor mit 3 Promotor-Terminator- Expressionskassetten inseriert mit Physcomitrella Elongase + Desaturase + Phaeodactylum Desaturase

SEQ ID NO: 30 - protein, 525 aa, Unknown; identical to SEQ ID NO: 8
  pflanz. Expressionsvektor mit 3 Promotor-Terminator- Expressionskassetten inseriert mit Physcomitrella Elongase + Desaturase + Phaeodactylum Desaturase

SEQ ID NO: 31 - protein, 469 aa, Unknown; identical to SEQ ID NO: 2
  pflanz. Expressionsvektor mit 3 Promotor-Terminator- Expressionskassetten inseriert mit Physcomitrella Elongase + Desaturase + Phaeodactylum Desaturase

SEQ ID NO: 32 - DNA, 26 bp, Artificial Sequence
  Polylinker
gaattcggcgcgccgagctcctcgag

SEQ ID NO: 33 - DNA, 265 bp, Artificial Sequence
  Polylinker-Terminator-Polylinker
ccaccgcggtgggcggccgcctgcagtctagaaggcctcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccgtcgacgaattcgagctcggcgcgccaagctt

SEQ ID NO: 34 - DNA, 257 bp, Artificial Sequence
  Polylinker-Terminator-Polylinker
ggatccgatatcgggcccgctagcgttaaccctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccgtcgacgaattcgagctcggcgcgccaagctt

SEQ ID NO: 35 - DNA, 257 bp, Artificial Sequence
  Polylinker-Terminator-Polylinker
agatctgccggcatcgatcccgggccatggcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccgtcgacgaattcgagctcggcgcgccaagctt